(19) United States
(12) Patent Application Publication
(10) Pub. No.: US 2020/0008299 A1
(43) Pub. Date: Jan. 2, 2020

(54) FLEXIBLE PRINTED ELECTRONICS

(71) Applicant: Bao Tran, (US)

(72) Inventors: Bao Tran, Saratoga, CA (US); Ha Tran, Saratoga, CA (US)

(21) Appl. No.: 16/267,253

(22) Filed: Feb. 4, 2019

Related U.S. Application Data
(63) Continuation-in-part of application No. 15/299,460, filed on Oct. 21, 2016.

(51) Int. Cl. H05K 1/03; H05K 3/10; H05K 1/02; A61B 5/00

(52) U.S. Cl. CPC H05K 1/0393; H05K 3/10; A61B 5/686; H05K 1/0386; H05K 1/0286; H05K 1/0313

(57) ABSTRACT

A biodegradable system includes a biodegradable substrate which can be a biodegradable paper or polymer. A biodegradable power source is printed or deposited above the substrate, and biodegradable processor and communication circuits are in turn formed on the substrate. The processor and wireless communication system can communicate with a remote computer to provide information about the source of the items (optionally tracked using the blockchain supply chain tracking), the relevant dates (production and expiration dates), any attempt to tamper with the packaging, and suitable warnings or usage instructions, for example. Optionally, a biodegradable display can render information to a customer, for example.

This application is a CIP of application Ser. No. 15/299,460, filed Oct. 21, 2016, the content of which is incorporated by reference.

### BACKGROUND

The present invention relates to flexible printed electronics.

Flexible electronics, also known as flex circuits, is a technology for assembling electronic circuits by mounting electronic devices on flexible plastic substrates, such as polyimide, PEEK or transparent conductive polyester film. Additionally, flex circuits can be screen printed silver circuits on polyester. Flexible electronic assemblies may be manufactured using identical components used for rigid printed circuit boards, allowing the board to conform to a desired shape, or to flex during its use. An alternative approach to flexible electronics uses various etching techniques to thin down the traditional silicon substrate to few tens of micrometers to gain reasonable flexibility.

### SUMMARY

In one aspect:

A method to fabricate biodegradable electronics includes forming a biodegradable substrate; forming a biodegradable power; and forming a biodegradable processor, memory, and a wireless or optical communication circuit on the substrate.

Advantages of the system may include one or more of the following. Printed electronics enable new markets for large-area, flexible or low-cost disposable devices. Using printing to fabricate electronic devices achieves lower manufacturing costs because of the additive, non-vacuum nature of the technology and the advantage of roll-to-roll or large-area processes. Moreover, printing can have processing advantages as in the case of contact-less printing onto fragile substrates. The system enables low cost, high performance sensors, e.g. for medical applications, and integrated electronic circuits. Also printed photovoltaics and printed lighting can be done. Other advantages are detailed below.

### BRIEF DESCRIPTION OF THE DRAWINGS

FIG. 1 shows an exemplary printed Internet of Things (IoT) sensor.

FIG. 2 shows an exemplary cloud-based structure supporting sensors of FIG. 1.

FIG. 3 shows an exemplary flexible electronic device.

FIG. 4 shows exemplary functionalized nano-material such as CNTs.

FIG. 5A shows an exemplary flexible printed circuit with a micro-needle region.

FIG. 5B shows an exemplary flexible sensor array.

FIGS. 5C-5D show exemplary clothing with flexible circuits thereon.

FIG. 5E shows an exemplary diaper with flexible circuits thereon.

FIG. 5F shows an exemplary band-aid or patch with flexible circuits thereon.

FIG. 5G shows an exemplary contact lens with flexible circuits thereon.

FIG. 5H shows an exemplary eye glass with flexible circuits thereon.

FIGS. 5I-5J shows an exemplary quality assurance system for vegetable or medication packages that need to monitor a temperature range, for example.

FIG. 5K shows an exemplary large panel with flexible resistive heater circuits thereon.

FIG. 5L shows an exemplary active display billboard with flexible circuits thereon.

FIG. 5M shows exemplary floor tiles with flexible circuits thereon.

FIGS. 5N-5O show exemplary smart building exterior with flexible circuits thereon.

### DESCRIPTION

The following illustrative embodiments described in the detailed description, drawings, and claims are not meant to be limiting. In the drawings, similar symbols typically identify similar components, unless context dictates otherwise. Other embodiments may be utilized, and other changes may be made, without departing from the spirit or scope of the subject matter presented here.

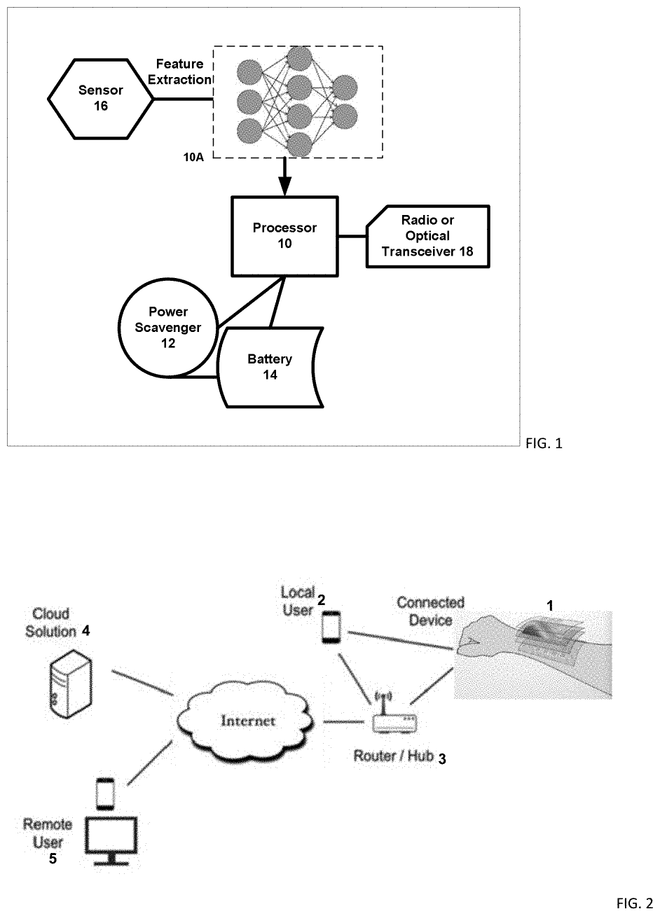

FIG. 1 shows an exemplary printed Internet of Things (IoT) flexible sensor device 1. The flexible sensor device 1 can have a flexible substrate 13 with a surface that is configured for receiving a flexible sensor 16. The flexible sensor 16 can be any flexible sensor or sensor circuit that can detect the presence of a target substance (a chemical compound) or electrical pattern (such as EKG or DNA, for example) or any other suitable tests. The substrate 13 can be made of a polymeric body and/or an inorganic-organic complex. Also, ceramics with suitable flexibility can be included in the substrate, as detailed below. The device is printed using low cost roll-to-roll manufacturing, inkjet printing or plasma jet fabrication, or a combination thereof, among others. In a complex sensor circuit, the device 1 can have a flexible substrate that is configured for receiving a first flexible sensor circuit electronically coupled to a second flexible sensor circuit. Such electronic coupling can be obtained, for example, an electronic path operatively linking a first flexible sensor circuit and a second flexible sensor circuit. The electronic coupling of flexible sensor circuits can be used to prepare more complex sensor systems. Also, any number of sensor circuits can be electronically coupled. The sensor circuits can be configured as described herein. In other embodiments, hybrid flexible electronics with part flexible circuit and part conventional circuits can be implemented.

One or more structures printed on the device can be a sensor 16 which captures information from the environment, such as temperature, EKG, DNA information, or glucose level, for example. The sensor can be a combination of sensors, nanowires, conductive polymers, and the like, and can include target recognition moieties for detecting target substances. While the raw data can be sent directly over the Internet via a wired or wireless connection, in one embodiment, the data is provided to an optional input pre-processor and then to a feature extractor/processor 10 which transforms raw data into a set of features to increase detection and minimize data transmission size/power consumption. The processor can be a conventional IC mounted on a printed motherboard, or the processor can be directly printed on the substrate. In one embodiment, the processor contains a general purpose processor communicating with a neural network 10A that can be trained to recognize patterns. The neural network 10A can have analog or digital implementations. In one embodiment, a pattern-matching recognition neural network is composed of 128 arithmetic units or neurons to perform two types of pattern recognition; the k-nearest neighbour (KNN) recognition and the radial basis function. Various desired patterns can be programmed and engine returns a positive match, uncertain, or negative match within a fixed time. The network is used as part of a wake-up system so that a sensor subsystem can pass a series of feature vectors to the neural network, which matches it against a stored dataset. If a wake up event is detect, the processor 10 is woken to decide whether to process information locally or to send information on to a sensor hub.

The sensor and processor 10 is powered by a power scavenger 12, an energy storage device 14, or a combination thereof. The scavenger 12 can be a printed antenna harvesting energy from FM stations, WiFi routers, cellular stations in one embodiment. The scavenger 12 can capture heat, sound, wind, or solar energy in other embodiments. The energy storage device 14 can be a printed supercapacitor or printed battery, among others.

The flexible substrate 13 can have any suitable shape or dimension along any vector. The flexible substrate 13 can also be a porous substrate. The pores (not shown) can extend, for example, from the surface into the substrate 13 or all the way through the substrate 13. Non-limiting examples of the shape of the substrate 13 can include a rectangle, block, triangle, amorphous shape, sphere, cube, polygon, and the like formed in three dimensions or as a substantially two dimensional sheet. The substrate can be any substrate known in the art.

A biodegradable system is detailed next. The system includes a biodegradable substrate which can be a biodegradable paper or polymer. A biodegradable power source is printed or deposited above the substrate, and biodegradable processor and communication circuits are in turn formed on the substrate. The processor and wireless communication system can communicate with a remote computer to provide information about the source of the items (optionally tracked using the blockchain supply chain tracking), the relevant dates (production and expiration dates), any attempt to tamper with the packaging, and suitable warnings or usage instructions, for example. Optionally, a biodegradable display can render information to a customer, for example.

In one embodiment, biodegradable silk can be used as the biodegradable substrate with high-performance inorganic semiconductors. Paper or Cellulose nanofibril (CNF) is an ecofriendly material as it is completely derived from wood. With its high transparency and flexibility, as well as desirable electrical properties, CNF can be an ecofriendly substrate for electronics. In other embodiments, biodegradable polymers (BDPs) can be bio-based or petrochemical-based. The former is mostly biodegradable by nature and produced from natural origins (plants, animals or micro-organisms) such as polysaccharides (e.g. starch, cellulose, lignin and chitin), proteins (e.g. gelatine, casein, wheat gluten, silk and wool) and lipids (e.g. plant oils and animal fats). Natural rubber as well as certain polyesters either produced by micro-organism/plant (e.g. polyhydroxyalkanoates and poly-3-hydroxybutyrate) or synthesized from bio-derived monomers (e.g. polylactic acid (PLA)) fall into this category. Petrochemical-based BDPs such as aliphatic polyesters (e.g. polyglycolic acid, polybutylene succinate and polycaprolactone (PCL)), aromatic copolyesters (e.g. polybutylene succinate terephthalate) and poly(vinyl alcohol) are produced by synthesis from monomers derived from petrochemical refining, which possess certain degrees of inherent biodegradability. Some BDP formulations combine materials from both classes to reduce cost and/or enhance performance.

Some BDP polymer blends that contain partly biogenic (renewable) carbon derived from biomass and partly petrochemical carbon. The biodegradability of a given polymer is effectively coupled with appropriate waste management in order to capture maximum environmental benefit.

One embodiment uses a microbial power source that is digitally printed onto paper substrate or a suitable BDP with Synechocystis cells as printed cyanobacteria. The bio-power source has a bioelectrode as the combination of photosynthetic organisms with an inert electrode material. The cyanobacterial bioelectrode can be printed in a two step process: firstly, the electrode is printed on the paper substrate using an inorganic conductive inkjet ink and secondly the cyanobacteria are printed onto the electrode pattern on the paper. The conductive inkjet ink can be the “Nink-1000: multiwall” (NanoLab, USA), which consists of carbon nanotubes (CNTs) in aqueous suspension.

In one embodiment, Synechocystis sp. PCC 6803 and the glucosetolerant wild type (WT-G)50 can be used. The WT-G strain was grown in BG-11 medium50 and Synechocystis PCC 6803 in BG-11 medium containing 3.6% (w/v) NaCl (BG-11 high salt) until mid-log phase, pelleted by centrifugation and resuspended in 1/100th the volume of fresh BG-11 medium. The concentrated cell resuspensions are reconstituted to form a ‘bioink’ in a Falcon tube and kept in the container till before the printing process. To grow the bacteria, a liquid culture of the cyanobacterium Synechococcus sp. PCC 7002 can be grown in medium A+49 supplemented with D7 micronutrients51, and cells concentrated as above. Agar plates contained BG-11 medium supplemented with 1.5% (w/w) agar. Cells are then grown at 30° C. at an irradiance of 20-30 μE m-2 s-1 of fluorescent white light (Sylvania Gro-Lux tubes). A plurality of power cells can be connected in series or in parallel to provide the required voltage or current.

In one embodiment, the microbes are coated with a shell that is dissolved by a liquid such as water. In this state, the microbes are dormant and can last several years to increase the shelf life of the system. Just before use, water is introduced to the microbes to dissolve the shell (water activation), and the microbes are then activated to generate electricity. This can be done by storing water on the substrate and at the right time, providing the water to the shelled microbes to dissolve the shell and activate the microbes for use.

As the microbial power sources produces low power even as an array, the collective power generated by the power sources are stored in biodegradable microsupercapacitors (MSCs) built using water-soluble (i.e., physically transient) metal (W, Fe, and Mo) electrodes, a biopolymer, hydrogel electrolyte (agarose gel), and a biodegradable poly(lactic-co-glycolic acid) substrate, encapsulated with polyanhydride. The pseudo-capacitance originates from metal-oxide coatings generated by electrochemical corrosion at the interface between the water-soluble metal electrode and the hydrogel electrolyte. The MSC works with the microbial power source as transient sources of power in the operation of light-emitting diodes and as charging capacitors in integrated circuits for wireless power harvesting.

The microbial power source in turn drives a biodegradable controller or processor. In one embodiment, GaInP/GaAs heterojunction bipolar transistors (HBTs) can be formed on a CNF substrate. Thin heterojunction epitaxial layers in stacks of n-cap layer (GaAs:Si)/n-emitter layer (GaInP:Si)/p-base layer (GaAs:C)/n-collector layer (GaAs:Si)/n-sub-collector layer (GaAs:Si) were grown on a 500-nm thick sacrificial layer (Al0.96Ga0.04As) on a GaAs wafer. The fabrication process began by following conventional procedures to fabricate the HBTs, followed by protective anchor patterning using a photoresist (PR) to protect the devices and allow the devices to be tethered to the substrate after etching away the underlying sacrificial layer using a diluted hydrofluoric acid (HF) solution. Van der Waals contact with a soft elastomer stamp made of polydimethylsiloxane (PDMS) to the device breaks the anchors on all four sides and easily picks up a single device. The devices are transfer printed in deterministic assembly onto a temporary Si substrate using ultrathin polyimide (PI, ˜1 μm) as an adhesive, followed by ground-signal-ground (G-S-G) RF interconnect metallization. PI material can be used for GaAs-based devices not only as an adhesive, but also as a passivating material that can suppress the high surface states of GaAs and prevent leakage current. Devices are then released from the temporary substrate and printed onto a CNF substrate using a PDMS stamp.

Optionally, a biodegradable display can be used. Organic LEDs (OLEDs) work in a similar way to conventional diodes and LEDs, but instead of using layers of n-type and p-type semiconductors, they use organic molecules to produce their electrons and holes. A simple OLED is made up of six different layers. On the top and bottom there are layers of protective glass or plastic. The top layer is called the seal and the bottom layer the substrate. In between those layers, there's a negative terminal (sometimes called the cathode) and a positive terminal (called the anode). Finally, in between the anode and cathode are two layers made from organic molecules called the emissive layer (where the light is produced, which is next to the cathode) and the conductive layer (next to the anode). To make an OLED light up, a voltage (potential difference) is achieved across the anode and cathode. As the electricity starts to flow, the cathode receives electrons from the power source and the anode loses them (or it “receives holes”). Positive holes are much more mobile than negative electrons so they jump across the boundary from the conductive layer to the emissive layer. When a hole (a lack of electron) meets an electron, the two things cancel out and release a brief burst of energy in the form of a particle of light or a photon. The display can be intermittently powered by microbial power source with biodegradable storage capacitor as power is saved up for displaying complex color images. Alternatively, black/white images can be rendered using biodegradable e-ink displays or LED displays.

The above system provides environmentally friendly manufacturing and retail operating using biodegradable food and medicine wrappers with degradable electronics including processor, communication, battery and display to indicate the expiration date and usage instructions, for example.

In one aspect, the system of FIG. 1 can use the following process to fabricate biodegradable electronics by forming a biodegradable substrate; forming a biodegradable power; and forming a biodegradable processor, memory, and a wireless or optical communication circuit on the substrate.

The system can be a biodegradable paper or biodegradable polymer. The method includes printing or depositing components above the substrate. The processor and wireless communication system can communicate with a remote computer to provide information about the source of the items and storing supply chain information on the substrate. The method includes storing production or expiration information on the substrate; storing tamper-proof information on the substrate; storing blockchain information on the substrate; forming a biodegradable display on the substrate; forming a bacteria storage on the substrate; forming a biodegradable food storage coupled to the bacteria storage on the substrate; forming a liquid storage coupled to the bacteria storage on the substrate, wherein liquid is selectively introduced to the bacteria to activate the bacteria to provide energy; detecting a predetermined substance. The method includes functionalizing a macromolecule with a material to couple to a predetermined substance; forming the functionalized macromolecule on a flexible substrate; exposing the macromolecule to an operating environment to attach the macromolecule to the predetermined substance; measuring an electrical characteristic indicative of the presence of the predetermined substance; and indicating a presence of the substance if the electrical characteristic is greater than or less than a predetermined range. The method further includes securing the macromolecule to a skin to capture sweat; detecting one or more of metabolite, glucose, lactate, electrolyte, sodium, potassium. The substance comprises a bio-marker, comprising exposing the macromolecule to blood. The macromolecule can be secured to a skin with microneedles to expose the macromolecule to subdermal blood. An implantable medical device can be formed with an exposed region to expose the macromolecule to blood and implanting the device inside a person. The bio-marker comprises one or more cancer biomarkers, further comprising detecting cancer from DNA fragments circulating in the blood and wherein the material comprises a predetermined DNA sequence, further including functionalizing the macromolecule with a second material to bond with a second DNA sequence complementary to the predetermined DNA sequence; during operation, generating a complementary DNA sequence from cell material in the blood and coupling the complementary DNA sequence to the second material; characterizing a second electrical characteristic indicative of the presence of the second DNA sequence, applying differential analysis to the first and second electrical characteristics to accurately determine a presence of the predetermined DNA sequence; and detecting the presence of the bio-marker using machine learning.

FIG. 2 shows an exemplary cloud-based structure supporting sensors of FIG. 1. A connected flexible printed device 1 such as the sensor of FIG. 1 is connected (wired or wireless) to a router/hub 3. The router/hub 3 transmits to the Internet to a cloud solution 4 which can provide storage of data flowing from the connected sensor of FIG. 1, or can include complex analytic functions that are performed on the data coming from the device and reported to a local user 2 or remote user 5. The local user 5 can interact directly with the sensor device 1 to either control it, or receive information regarding its operation. The router connects the device 1 to the Internet with a suitable modem using fiber optic, ADSL, cable, cellular, among others. The remote user 5 is not in the proximity of the device and can control or receive information regarding the device from afar. One embodiment sends data to the Cloud using NFC or Bluetooth and then use the local user's smartphone as their hub to the Internet, or a special hub can be provided that routes the Bluetooth data through Ethernet/Wi-Fi/cellular to the Internet. Wi-Fi, a more power-hungry solution, but still relatively low power, can be used for devices that are connected to external power, or can be charged periodically. Wi-Fi, in contrast to Bluetooth, can connect to the Internet and the Cloud directly via an existing Wi-Fi router without a special hub required. If Ethernet (LAN) is available where the device is located and the device is stationary, a wired connection may be a good choice—it is usually the lowest cost and simplest connectivity method for the device.

Electrically functional inks are deposited on the substrate, creating active or passive devices, such as thin film circuits, sensors, transistors or resistors. The term printed electronics specifies the process and can utilize any solution-based material. The use of flexible electronic printing enables low-cost volume fabrication which has opened the door for the medical industry to include electrically functional parts as disposables. Printed electronics offer reliability as well as patient comfort, less invasiveness and can be disposable, with the ability to offer remote diagnostics in a cost effective, disposable form is driving use of printed electronics. Biosensors such as EKG/ECG electrodes, glucose test strips and pads for drug delivery manufactured by using combinations of silver, silver-silver chloride, carbon, and di-electric inks printed on thin film polyester have become the norm.

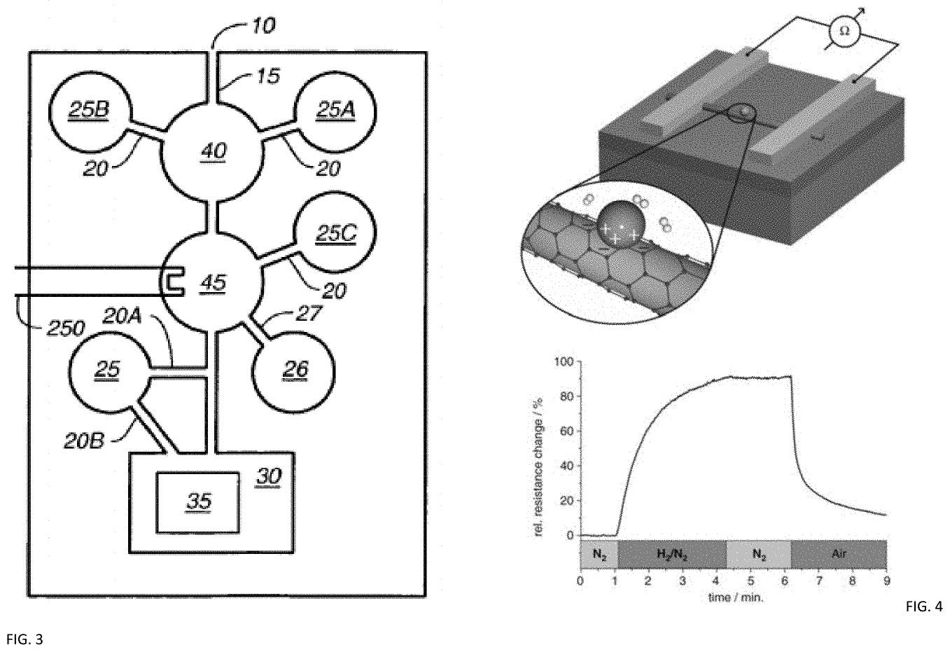

FIG. 3 shows an exemplary printed genetic lab on a chip with a sample inlet port 10, a first channel 15, a storage module 25 (for example, for assay reagents) with a second channel 20B. The second channel (20B) may be in fluid contact directly with the detection module 30 comprising a detection electrode 35, or (20A) in contact with the first channel 15. Also shown is a sample handling reservoir 40 and a second storage reservoir 25A with a channel 20 to the sample handling reservoir 40. For example, the sample handling reservoir 40 could be a cell lysis chamber and the storage reservoir 25A could contain lysis reagents. A sample handling reservoir 40 can be a cell capture or enrichment chamber, with an additional reagent storage reservoir 25B for elution buffer. A reaction module 45 can be used with a storage module 25C, for example for storage of amplification reagents. Optional waste module 26 is connected to the reaction module 45 via a channel 27. All of these embodiments may additionally comprise valves, waste reservoirs, and pumps, including additional electrodes. In practice, a flexible substrate may comprise one or more reservoirs and one or more channels. When the substrate comprises a plurality of channels, the channels may be connected to, and extend from, the same or respective reservoirs. Furthermore, each channel may be configured to enable the formation of an electrical connection to the same connector of the appropriately positioned electronic component or a different connector of the appropriately positioned electronic component.

In one embodiment, electrophoresis can be used to move a solution through one or more channels into predetermined well sequences. Electrophoresis is the motion of dispersed particles relative to a fluid under the influence of a spatially uniform electric field. The application of a constant electric field caused clay particles dispersed in water to migrate. It is ultimately caused by the presence of a charged interface between the particle surface and the surrounding fluid. The system can apply electrophoresis or any other suitable techniques for separating molecules by size, charge, or binding affinity. Electrophoresis of positively charged particles (cations) is called cataphoresis, while electrophoresis of negatively charged particles (anions) is called anaphoresis. Electrophoresis is a technique used in laboratories in order to separate macromolecules based on size. The technique applies a negative charge so proteins move towards a positive charge. This is used for both DNA and RNA analysis. Polyacrylamide gel electrophoresis (PAGE) has a clearer resolution than agarose and is more suitable for quantitative analysis. In this technique DNA foot-printing can identify how proteins bind to DNA. It can be used to separate proteins by size, density and purity. It can also be used for plasmid analysis, which develops our understanding of bacteria becoming resistant to antibiotics.

In one embodiment, the reservoirs and channels can be used to attach a conventional silicon die to the flexible substrate. The reservoir can be shaped to receive a component end or die pad and the well can be filled with a conductive fluid that when cured, can enable the formation of an electrical connection to the die or electronic component. Formation of the electrical connection allows the die to be interconnected to other electronic components using one or more of the reservoir and channel filled with conductive ink or a printed wire coupled to the reservoir.

The reservoirs and channels are formed in the substrate using one or more of hot-embossing, laser ablation, nanoimprinting, photolithography, and casting a substrate material as a solution over a mould before curing. The substrate itself may comprise one or more of polyethylene naphthalate (PEN), polyethylene terephthalate (PET), polyimide (PI), polycarbonate (PC), polydimethylsiloxane (PDMS) and polyurethane (PU).

A printing or coating process (e.g. one or more of inkjet printing, flexographic printing, gravure printing, aerosol jet printing, dip coating and slot coating) may be used to deposit reagents, hydrogels, or fluids into the reservoirs. The channels may be configured to guide the fluid to/from the reservoirs using one or more of capillary action, Laplace pressure, fluidphilic interaction and fluidphobic interaction. Capillary action refers to the spontaneous “wicking” of the electrically conductive fluid along the axis of the channel due to the combination of surface tension within the fluid and adhesive forces between the fluid and the channel.

In some embodiments, the reservoirs may comprise a fluidphobic material and/or the channels may comprise a fluidphilic material to facilitate guiding of the electrically conductive fluid from the reservoirs to the component region of the substrate. The term “fluidphobic” may be taken to mean any material which is capable of repelling a fluid, and may encompass hydrophobic, lipophobic (oleophobic) and lyophobic materials. Likewise, the term “fluidphilic” may be taken to mean any material which is capable of attracting a fluid, and may encompass hydrophilic, lipophilic (oleophilic) and lyophilic materials. A region of the substrate surrounding each channel may comprise a fluidphobic material configured to guide any electrically conductive fluid deposited within this region into the channel.

The structure of a channel also affects the ability of the channel to transport fluid. For example, the size, shape and channel angle of the channel can each influence its transport properties. In this respect, one or more of the size and shape of the channels may be configured to guide the electrically conductive fluid from the reservoirs to the component region. A combination of capillary action and Laplace pressure can be used to guide the fluid to/from the reservoir. The channels may have a number of different profiles. For example, each channel may have a triangular, square, rectangular, symmetric trapezoidal, asymmetric trapezoidal, or concave profile.

Generally, flexible sensor devices and compositions for making and using the same can be used for detecting the presence of a target substance. The flexible sensor devices and compositions can be configured to include various concentrations or amounts of flexible sensors that interact with the target substance to provide a detectable signal as an indication of such an interaction. The flexible sensor device can be achieved by placing one or more sensors or sensor circuits onto a flexible substrate that holds and retains the one or more sensors or sensor circuits. The flexible substrate can have various configurations that provide for sufficient flexibility for an intended use while retaining the functionality of the one or more sensors or sensor circuits. Discussions of sensors are intended also to refer to sensor circuits and vice versa.

A flexible sensor device can be configured to be used for detecting a target substance in a medium. The flexible sensor device can include a flexible substrate, and at least one flexible sensor included and retained on the flexible substrate. The sensor can be configured to interact with a target substance so as to provide a signal that can be detected. The target substance can be any type of substance. Non-limiting examples of a suitable target substance can include an organic molecule, inorganic molecule, atom, ion, nucleotide, polynucleotide, amino acid, polypeptide, protein, receptor, antibody, antibody fragment, cell, cell surface component, ligand, combinations thereof, or the like. When the target substance is a target polynucleotide, the sensor can include a probe polynucleotide configured to hybridize with the target polynucleotide. When the target substance is a target polypeptide, the sensor can include a target recognition moiety configured to interact with the target polypeptide. When the target substance is a target cell, the sensor can include a target recognition moiety configured to interact with a cell surface component of the target cell. Non-limiting examples of cell surface components include a protein, epitope, receptor, cell membrane component, lipid, combinations thereof, or the like.

In one embodiment, a flexible sensor device that detects polynucleotides can include at least one flexible sensor that detects polynucleotides included and retained on a flexible substrate. The flexible sensor can include a probe polynucleotide configured to hybridize with a target polynucleotide. Also, the probe polynucleotide of the nanosensor can have a high degree of specificity for the target polynucleotide, the high degree of specificity being characterized by at least 90% complementarity.

As used herein, the terms “complementary” and “complementarity” are meant to refer to the ability of polynucleotides to form base pairs with one another. Base pairs are typically formed by hydrogen bonds between nucleotide units in anti-parallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine.

Perfect complementarity or 100% complementarity refers to the situation in which each nucleotide unit of one polynucleotide strand can hydrogen bond with a nucleotide unit of an anti-parallel polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. “Substantial complementarity” refers to polynucleotide strands exhibiting 79% or greater complementarity, that are selected so as to be non-complementary.

In one embodiment, a flexible sensor device that detects polypeptides can include at least one flexible sensor that detects polypeptides included and retained on a flexible substrate. The sensor can include a target recognition moiety configured to interact with a target polypeptide. The target recognition moiety can be, but is not limited to, one of a polypeptide, protein, receptor, antibody, antibody fragment, ligand, combinations thereof, or the like. The target recognition moiety can be selected and/or configured to interact with the target poloypeptide in any possible condition or manner.

In one embodiment, a flexible sensor device that detects cells can include at least one flexible sensor that detects cells included and retained on a flexible substrate. The sensor can include a target recognition moiety configured to interact with a cell surface component of a target cell. Non-limiting examples of a cell surface component include a protein, epitope, receptor, cell membrane component, lipid, combinations thereof, or the like. The target recognition moiety can be selected and/or configured to interact with the target cell in any possible condition or manner.

The flexible substrate can be prepared from any polymer. This can include non-biocompatible polymers as well as biocompatible polymers. In one instance, the biocompatible polymer can be a biostable polymer. In another instance, the biocompatible polymer can have a degree of biodegradability. Non-limiting examples of general polymers that can be configured for suitable flexibility for use in a flexible sensor device can include: polyethylenes, polyethylene (PE), Low density polyethylene (LDPE), high density polyethylene (HDPE), crosslinked polyethylene (XLPE); polypropylenes, polypropylene (PP), polybutylene (PB), polyisobutylene (PIB), biaxially-oriented polypropylene; polyarylates, polymethyl methacrylate (PMMA), polymethyl acrylate (PMA), hydroxyethyl methacrylate (HEMA), polybutadiene acrylonitrile (PBAN), sodium polyacrylate polyacrylamide (PAM); polyesteres, polystyrene (PS), polyethylene terphthalate (PET), acrylonitrile butadiene styrene (ABS), high impact polystyrene (HIPS), extruded polystyrene (XPS); polysulphones, polysulfone (PSU), polyarylsulfone (PAS), polyethersulfone (PES), polyphenylsulfone (PPS); polyamides (PA), polyphthalamide (PPA), bismaleimide (BMI), urea formaldehyde (UF); polyurethanes (PU), polyisocyanurate (PIR); polyvinyls, polyvinyl chloride (PVC), polyvinylidene chloride (PVDC); fluoropolymers, fluoroethylene (FE), polytetrafluoroethylene (PTFE); ethylene chlorotrifluoroethlyene (ECTFE); polycarbonate (PC), polylactic acid (PLA), and the like. Non-limiting examples of biocompatible polymers that can be used in the flexible sensor device can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, copolymers thereof, derivative polymers thereof, monomers thereof, combinations thereof, or the like. Other biocompatible, biodegradable, and/or biostable polymers can be used with or in place of any of the above-referenced polymers. The flexible substrate can also be water stable so that the container body does not degrade in the presence of water or other aqueous solution. Also, the flexible substrate can be prepared from polymers that have stability in organic solutions so that the flexible sensor device does not degrade when in an organic solution, organic components, or hydrophobic components.

Non-limiting examples of inorganic-organic complexes that can be included in flexible substrates can include: flexible ligand 1,3-bis(4-pyridyl)propane with Co(NCS)2.xH2O; a combination of a sulfonate salt and an alkaline inorganic metal salt, whereby the crystalline structure of the inorganic portion of the complex is platelet and film-forming in character.

The flexible sensor 13, as shown in FIG. 1, can be any sensor or combination of sensors as well as sensor circuits. The flexible sensor 13 can be a single sensor or a combination of sensors, such as combination of nanosensors. The sensor 13 can be configured to detect a chemical substance, such as but not limited to, organic molecule, inorganic molecule, atom, ion, nucleotide, polynucleotide, amino acid, polypeptide, protein, receptor, antibody, antibody fragment, cell, cell surface component, ligand, combinations thereof, or the like.

In one embodiment, the sensor or sensor circuit can be configured to detect a target polynucleotide. Such a sensor can include a probe polynucleotide that is configured for hybridizing or otherwise associating with a target polynucleotide. The interaction between the probe polynucleotide and the target polynucleotide can provide a signal that can be detected. The probe polynucleotide can have a high degree of specificity for the target polynucleotide, the high degree of specificity being characterized by at least about 75%, at least about 90%, or at least about 99% complementarity of the target polynucleotide with the probe polynucleotide, or about 50% to about 75%, about 75% to about 90%, or 90% to about 99% complementarity. The interaction between the target polynucleotide and probe polynucleotide can provide a signal that is selected from the group consisting of an electronic signal, optical signal, magnetic signal, electrochemical signal, and combinations thereof. Also, the interaction between the target polynucleotide and probe polynucleotide of the nanosensor can induce a detectable change in the signal.

In one embodiment, the sensor or sensor circuit can be configured to detect a target polypeptide. Such a sensor can include a target recognition moiety configured for binding, associating, or interacting with a target polypeptide. The target recognition moiety can be, for example without limitation, a protein, receptor, antibody, antibody fragment, or the like that interacts with a target polypeptide. The sensor can have a high degree of specificity for the target polypeptide, wherein high specificity can be characterized by the target recognition moiety only interacting with the target polypeptide, medium specificity can be characterized by the target recognition moiety interacting with the target polypeptide and derivatives and analogs thereof, and low specificity can be characterized by the target recognition moiety interacting with a genus of polypeptides that include the target polypeptide as a species thereof. Also, the interaction between the sensor can provide a signal selected from the group consisting of an electronic signal, optical signal, magnetic signal, electrochemical signal, and combinations thereof. Also, the interaction between the target recognition moiety and the target polypeptide can induce a detectable change in the signal.

In one embodiment, the sensor or sensor circuit can be configured to detect a target cell. Such a sensor can include a target cell recognition moiety (e.g., protein, receptor, antibody, antibody fragment, ligand, etc.) that interacts with a cell surface component of the target cell. Non-limiting examples of a cell surface component include a protein, epitope, receptor, cell membrane component, lipid, combinations thereof, or the like. The sensor can have a high degree of specificity for the target cell, wherein high specificity can be characterized by the target recognition moiety only interacting with the target cell, medium specificity can be characterized by the target recognition moiety interacting with the target cell and other similar cell types, and low specificity can be characterized by the target recognition moiety interacting with a genus of cells that include the target cell as a species thereof. Also, the interaction between the sensor can provide a signal selected from the group consisting of an electronic signal, optical signal, magnetic signal, electrochemical signal, and combinations thereof. Also, the interaction between the target recognition moiety and the target polypeptide can induce a detectable change in the signal.

The sensors and/or sensor circuits that can be included in the flexible sensor devices described herein represent a broad class of sensors that can be employed to detect a target substance. The sensors can include those described herein as well as those well known in the art and those later developed.

A nanowire is a wire of a diameter of the order of a nanometer, and can be defined as structures that have a lateral size constrained to tens of nanometers or less and an unconstrained longitudinal size. Many different types of nanowires exist, including metallic nanowires (e.g., Ni, Pt, Au, etc.), semiconducting nanowires (e.g., Si, InP, GaN, etc.), and insulating nanowires (e.g., SiO2, TiO2, etc.). Molecular nanowires can include repeating molecular units including either organic (e.g. DNA, RNA, etc.) or inorganic (e.g. Mo6S9-xlx) components. Nanowires can have aspect ratios of about 1000 or more. As such, nanowires can be referred to as 1-Dimensional materials. Electrons in nanowires are quantum confined laterally, and thus occupy energy levels that are different from the traditional continuum of energy levels or bands found in bulk materials. Quantum confinement of certain nanowires, such as carbon nanotubes, can provide electrical conductance. Non-limiting examples of nanowires can include inorganic molecular nanowires (e.g., Mo6S9-xlx, Li2Mo6Se6), which have a diameter of 0.9 nm, and can be hundreds of micrometers long. Additional non-limiting examples of nanowires can be based on semiconductors (e.g., InP, Si, GaN, etc.), dielectrics (e.g. SiO2, TiO2), or metals (e.g. Ni, Pt).

Nanowires can be used to fabricate sensor circuits by chemically doping a semiconductor nanowire to create p-type and n-type semiconductors. Also, a p-n junction, one of the simplest electronic devices, can be prepared by physically crossing a p-type wire over an n-type wire or chemically doping a single wire with different dopants along the length. Additionally, nanowires can be fabricated into logic gates by connecting several p-n junctions together, which provide a basis for all logic circuits: the AND, OR, and NOT gates can be prepared from semiconductor nanowire crossings.

In one embodiment, a sensor circuit can include a conducting polymer. Conducting polymers are configured to allow electrons to flow across so as to be electrically conductive. The conducting polymers can be used to prepare sensor circuits similarly to the use of conducting materials in circuits. Non-limiting examples of conducting polymers that can be used to prepare sensor circuits can include: conductive polypyrrole; high conductivity oxidized iodine-doped polypyrrole, a polyacetylene derivative; poly(phenylene vinylene) (PPV), which is an alternating copolymer of polyacteylene and poly(paraphenylene) can be a semiconducting polymer; poly(3-alkylthiophenes); a self-doped mixed copolymer of oxidized polyacetylene, polypyrrole and polyaniline having near metallic conductivity; organic conductive polymers, poly(acetylene), poly(pyrrole), poly(thiophene), poly(aniline), poly(fluorene), poly(3-alkylthiophene), polytetrathiafulvalene, polynaphthalene, poly(p-phenylene sulfide), poly(para-phenylene vinylene); malanins; derivatives thereof; combinations thereof; or other conducting polymers.

In one embodiment, a sensor or sensor circuit includes a molecule or ion sensor. Such molecular sensors can be configured to detect the presence of specific substances, and combine the properties of supramolecular receptors, as they specifically recognize a specific substance, with the ability to produce a measurable signal. Optical signals based on changes of absorbance, transmission, or fluorescence are the most frequently utilized because of their simple applications and use of common instruments. The molecular sensors can change absorbance, particularly of color, when interacting with a target substance. Such changes can be used to detect the presence of the target substance. The use of molecular sensors that provide or change fluorescence emission provides very high sensitivity of the sensor device. One category of fluorescence chemosensors includes classical fluorescence chemosensors made from molecules in which a supramolecular receptor and a fluorescence dye are part of the same molecule. Another class is that of self-organized fluorescence chemosensors, which are obtained by the spontaneous self-organizing of the sensor components.

A fluorescence chemosensor, ATMCA, can be obtained by coupling an anthrylmethyl group to an amino nitrogen of TMCA (2,4,6-triamino-1,3,5-trimethoxycyclohexane), a tripodal ligand selective for divalent first-row transition metal ions in water. The ATMCA ligand can act as a versatile sensor for Zn and Cu ions, where the sensing ability can be switched by simply tuning the operating conditions. At pH 5, ATMCA detects copper ions in aqueous solutions by the complexation-induced quenching of the anthracene emission. Metal ion concentrations <1 μM can be readily detected and very little interference is exerted by other metal ions. At pH 7, ATMCA signals the presence of Zn ions at concentrations <1 μM by a complexation-induced enhancement of the fluorescence. Such a chemosensor is a nanosensor, and can be used in the sensor devices as described herein.

Additionally, the [Zn(ATMCA)]2+ complex can act as a fluorescence nanosensor for specific organic species, such as selected dicarboxylic acids and nucleotides, by the formation of ternary ligand/zinc/substrate complexes. The oxalate anion can be detected in concentrations <0.1 mM. Nucleotides containing an imide or amide function can be detected with the nanosensor, and the nanosensor has high sensitivity for guanine derivatives. Moreover, the ATMCA.Zn(II) complex is an effective and selective sensor for vitamin B13 (orotic acid) in sub-micromolar concentrations. The formation of the complex with vitamin B13 leads to the quenching of the fluorescence emission of anthracenyl residue.

Another non-limiting example of a nanosensor is a Foster resonance energy transfer (FRET) amplified chemosensor. The sensing activity includes the binding of AI(III) to a 3,5-bis(ortho-hydroxyphenyl)-1,2,4-triazole group, and produces a chelation induced fluorescence enhancement (CHEF). The 3,5-bis(ortho-hydroxyphenyl)-1,2,4-triazole group can be used as a sensor as described herein. Also, conjugation of the 3,5-bis(ortho-hydroxyphenyl)-1,2,4-triazole group with coumarine 343 allows the amplification of the fluorescence signal via a FRET process.

Another non-limiting example of a nanosensor is a self-assembled chemosensor for Cu(II) having decylglycylglycine and ANS chromophore in close proximity. The Cu(II) selective receptor (decylglycylglycine) and a chromophore (ANS) can be in close proximity with CTABr surfactant so as to aggregate. Also, the components can be coupled to a microparticle, such as silica. The close proximity produces fluorescence quenching after Cu(II) addition in concentrations below the micromolar range. Commercially available particles (e.g., 20 nm diameter) can be functionalized with triethoxysilane derivatives of selective Cu(II) ligands and fluorophores. The sensor components can be coupled to the particle surface to provide spatial proximity to signal Cu(II) by quenching of the fluorescence emission. In 9:1 DMSO/water solution, the coated silica nanoparticles (CSNs) selectively detect copper ions down to nanomolar concentrations, and the operative range of the nanosensor can be tuned by the simple modification of the components ratio.

A tren-based tripodal chemosensor bearing a rhodamine and two tosyl groups can be prepared as a sensor to detect metal ions. Detection can be observed through UV/vis and fluorescence spectroscopies. Addition of a Hg2+ ion to the nanosensor can provide a visual color change as well as significantly enhanced fluorescence, while other ions including Pb2+, Zn2+, Cu2+, Ca2+, Ba2+, Cd2+, Co2+, Mg2+, Ag+, Cs+, Li+, and Na+ induced no or much smaller color/spectral changes. As such, the sensor is an Hg2+-selective fluorescent sensor. Such a nanosensor can be used as described herein.

Additionally, quantum dots or barcode quantum materials having specific arrangements and fluorescent augmentations can be used in a nanosensor. Zinc sulfide quantum dots, though not quite as fluorescent as cadmium selenide quantum dots, can have augmented fluorescence by including other metals such as manganese and various lanthanide elements. The quantum dots can become more fluorescent when they bond to their target, such as target substances, polynucleotides, polypeptides, and cells. The quantum dots or barcode quantum materials having the quantum dots can be used in ultrasensitive nanosensors. Different high-quality quantum dot nanocrystals (ZnS, CdS, and PbS) can be tagged to a target recognition moiety (e.g., probe polynucleotides, ligands, receptors, antibodies, antibody fragments, etc.) for on-site voltammetric stripping measurements of multiple antigen targets. The quantum dots or barcode quantum materials can have distinct redox potential and yield highly sensitive and selective stripping peaks at −1.11 V (Zn), −0.67 V (Cd) and −0.52 V (Pb) at a mercury-coated glassy carbon electrode compared to references. The change in position and size of these peaks reflect the presence and concentration level of the corresponding target.

A nanosensor can include a nanotube having a target recognition moiety that interacts with a target substance, polynucleotide, polypeptide, or cell. Accordingly, the target recognition moiety is configured for interacting with the target. The nanotube, such as a carbon nanotube, can have a first vibrational energy when the target recognition moiety is not interacting with the target and then have a second vibrational energy when the target recognition moiety interacts with the target. The difference between the first and second vibrational energy is measurable and detection of the difference can provide an indication that the target is present. Thus, any type of target recognition moiety can be applied to a nanotube in order to have a sensor that can be used as described herein. Energies other than vibrational energy may also be used for detection purposed.

In one embodiment, a nanosensor can be configured as a “core-satellite” structure, which resembles a planet (gold) with numerous smaller moons (particles) tethered to it by tiny strands of polynucleotides having probe polynucleotide sequences. The probe polynucleotide sequences can be configured for hybridizing with the target polynucleotide so as to have suitable complementarity. Gold core particles and smaller satellite particles of various materials are mixed together in solution with the probe polynucleotides and under controlled circumstances assemble themselves into the desired core-satellite structure. Following assembly, the structures are can be used to detect new strands of polynucleotides of various lengths. The probe polynucleotide tethers between the gold core and particles contract or expand when in the presence of the target polynucleotide. As the particles move in relation to the gold core, the optical properties of the structure change, and thereby provide a signal that can be detected.

In one embodiment, a nanosensor can be a bio-barcode nanosensor. A bio-barcode nanosensor includes a nanosensor that includes a series of barcode oligonucleotides. The barcode oligonucleotides can correspond to a specific target, and interaction of the target with the nanosensors releases one or more of the bio-barcodes, which can be detected.

In one embodiment, a nanosensor can include a nano-gap capacitor. Nan-gap capacitors can be fabricated using silicon nanolithography. A target recognition moiety is immobilized on the nano-gap capacitor in a manner that allows for interaction with the target substance. When the target substance interacts with the target recognition moiety, the capacitance changes in a detectable manner. As such, the nano-gap capacitor is configured to change the detected signal upon interaction of the target substance and a nanosensor.

In one embodiment, a nanosensor can include a nano-cantilever. A target recognition moiety is immobilized on the nano-cantilever in a manner that allows for interaction with the target substance. When the target substance interacts with the target recognition moiety, the deflection properties, vibrational properties, or response to probe signals changes in a detectable manner. Thus, a nano-cantilever can be coupled to a target substance recognition moiety such that interaction of the target substance and the recognition moiety changes the detected signal of the nano-cantilever.

In one embodiment, a sensor system can include any sensor device as described herein that includes a nanosensor in a polymeric container as described herein, and can include a monitor configured to detect a signal that indicates the nanosensor has sensed the target substance. The monitor can be selected based on the type of signal provided by the nanosensor. Printed piezonresistive sensors, piezoelectric sensor, microfluidic sensors, and displays can be formed, as well as gas sensors and hybrid organic image sensors.

The flexible sensors or sensor circuits on the flexible substrate can be configured to have various shapes and sizes over a broad range. With regard to size, the flexible sensors or sensor circuits can have a dimension, such as diameter, width, length, height, or the like, that ranges from about 10 nm to about 1 mm. In another option, the dimension can range from about 50 nm to about 100 um. In yet another option, the dimension can range from about 75 nm to about 10 um. In still yet another option, the dimension can range from about 100 nm to about 1 um. Also, larger flexible substrates can range between the foregoing values in the micrometer (um) range, millimeter (mm) range, and centimeter (cm range), or larger if needed. In some instances certain applications can utilize flexible sensors or sensor circuits that are larger, equal to, or smaller than any of the recited dimensions.

The flexible sensors or sensor circuits can have a high degree of specificity for the target substance. This can include the flexible sensors or sensor circuits being specific for the target substance so that the signal is provided only when the flexible sensors or sensor circuits interacts with the target substance, which is an example of strict specificity. Also, less stringent specificity can be used where the flexible sensors or sensor circuits provides the signal when it interacts with the target substance or a close derivative, analog, salt, or other minor change. Loose specificity can be used when the flexible sensors or sensor circuits provides a signal when interacting with one of a member of a class or a species of a genus of types of target substances.

Flexible sensors or sensor circuits can be configured to provide a signal that is selected from the group consisting of an electronic signal, optical signal, magnetic signal, electrochemical signal, and combinations thereof. Accordingly, a flexible sensors or sensor circuits can be selected or manufactured based on the type of signal provided. In different instances, any of the above-references signal types can be favorable. The selection of the flexible sensors or sensor circuits may result in a specific type of signal in instances where the flexible sensors or sensor circuits interact with a target substance to provide a specific signal type.

The flexible sensors or sensor circuits can provide a signal having a first characteristic in the absence of the target substance and then change the signal to a second characteristic upon interaction with the target substance. This can include a first wavelength or first wavelength pattern that is changed to a second wavelength or second wavelength pattern. The signal can have an absorption, transmission, or other emission profile that has a first characteristic, and the characteristic is changed to a second characteristic upon interaction with the target substance. Such a change can be detectible so that the detection of the targets substance results from detection in a change in the signal from a first characteristic to a second characteristic.

The flexible sensor device having the flexible sensors and/or sensor circuits can be configured for any degree of flexibility. This can include having sufficient flexibility to be bent from being flat to 180 degrees so as to be folded over itself. Also, the flexible sensor device can be rolled into a sleeve, tube, or the like. Additionally, the flexible sensor device can be configured to have sufficient flexibility to be included in a garment in any location of the garment, such as locations at the knee, buttocks, waste, abdomen, armpits, shoulders, elbows, and the like. Accordingly, the flexible sensor device and/or the flexible sensors and/or flexible sensor circuits can have any degree of elongation, contraction, and/or distortion. For example, without limitation, the flexibility can allow for elongation and/or distortion so as to change a dimension, such as length, width, height, diameter, or the like by about 110%, about 135%, about 150%, about 175%, about 200%, about 500%, or to about 1000% of the original value of the dimension, wherein 100% would be considered no change. In another non-limiting example, the contraction and/or distortion can change a dimension by about 90%, about 80%, about 75%, about 60%, about 50%, about 30%, about 25%, about 15%, or about 10% of the original value.

In one embodiment, a method of detecting a target substance with a flexible sensor device can be performed with a flexible sensor device as described herein that includes a flexible sensor or sensor circuit. The flexible sensor device can be placed in a medium to determine whether or not the target substance is present. When the sensor or sensor circuit of the flexible sensor device interacts with a target substance, a signal is provided. As such, detecting the signal provides an indication that the presence of the target substance in the medium. Optionally, the medium can be selected from the group consisting of water, air, biological sample, hydrocarbon, skin, tissue, body fluids, combinations thereof, and other similar media.

Additionally, the method can further include tagging the target substance with a marker that interacts with the sensor device so as to provide the signal. In various systems, a donor and acceptor can be used as a marker pair, where the target substance is modified to include one of the donor and acceptor and the sensor has the other. Close proximity or association of the donor and acceptor provides the detectable signal. For example, a target nucleic acid can be tagged with the marker, which is either the donor or acceptor, and the probe polynucleotide has the other. When the target hybridizes with the probe, the signal is provided.

The method of detecting a target substance can also include determining an amount or concentration of the target substance in the medium. Quantification of the signal or change in signal can be used to determine the amount or concentration of the target substance. Also, the signal can be compared to a control or control set in order to quantify or quantitate the amount or concentration of the target substance.

The method of detecting a target substance can include the use of a probe signal that induces the detection signal to be provided or to change the signal. As such, a probe signal can be directed into the medium to the nanosensor so as to induce at least one nanosensor to provide the signal. The probe signal can provide energy that is changed by the nanosensor in a detectable manner. For example, light of a broad or specific wavelength can be directed into the medium, and the obtained absorbance, transmittance, or fluorescence can be the signal provided as a result of the probe signal.

The sensor devices as described herein can be prepared by various methods of depositing, printing, or otherwise including a flexible sensor or flexible sensor circuit on a flexible substrate. The substrate can include a flexible polymer or inorganic-organic complex, which substrate can be porous in some instance. In other instances, the substrate can be substantially devoid of pores.

Circuits, antennas, and other electrical elements can be constructed on various types of substrates using, for example, laser direct structuring (LDS) and pad printing. LDS uses a laser beam to etch a pattern such as a circuit or antenna pattern into a thermoplastic material that is doped with an organic metal additive. A microscopically rough track is formed where the laser beam hits the doped thermoplastic material. The etched thermoplastic material is then subjected to a copper bath followed by metal plating. In pad printing, a pattern is etched into a plate that is subsequently filled with electrically conductive material. A pad is then placed onto the plate with enough pressure to transfer electrically conductive material to the pad. Finally, the pad is pressed onto a substrate transferring the electrically conductive material to the substrate in the shape of the etched pattern. This process is repeated several times to transfer a sufficient amount of electrically conductive material onto the substrate.

Thermal transferring techniques can be used to make electrically conductive materials. One method includes transferring an electrically conductive material to a substrate by contacting at least a portion of a substrate with electrically conductive material that is disposed on a carrier film. The carrier film may be made of any material that can withstand heat and pressure such that its function with the present methods is retained. For example, the carrier film used with the present methods may withstand heat applied during a hot stamping process such that the carrier film can transfer electrically conductive material to a substrate during a hot stamping process. The carrier film also may be flexible, allowing it to be contacted with substrates of varying dimensions and shapes. Non-limiting examples of suitable carrier films are films produced from polyethylene, polyethylene terephthalate (PET), polypropylene, polyesters, polyimides, polycarbonates, paper, impregnated paper, silicones, fluoropolymers, and copolymers and mixtures thereof. An example of a polyimide film that may be used as the carrier film is sold under the trade-name KAPTON®, which is commercially available from DuPont.

The electrically conductive material may be disposed over at least a portion of the carrier film in a pattern or design that, when adhered to a substrate, can be electrically connected to an electronic device by way of a conductive adhesive, electrically conductive pads, pogo-pins, vias or other methods, thus allowing an electrical current or signal to be transmitted to the electronic device. For instance, the electrically conductive material may be disposed over at least a portion of the carrier film in a pattern that forms a circuit or antenna. The electrically conductive material may be also disposed over at least a portion of the carrier film for the formation of piezo coils, electroluminescent, ground plane, and/or EMI/RFI shielding. When coupled to a pogo-pin, for example, an electrical connection can be made so that an electrical current or signal to be transmitted can be received or transmitted by the device. The electrically conductive material may be disposed, such as in a pattern, using various printing methods. Non-limiting examples of printing methods that can be used to apply the electrically conductive materials to the carrier film include digital printing, flexographic printing, gravure printing, screen printing, and the like.

After exposing the materials to an external source to promote drying, the dried material or materials can be exposed to ambient conditions before additional materials are applied. During this period of time, residual solvent still present after the drying step may continue to dissipate from the material or materials. The electrically conductive material, release coat, dielectric material, adhesive, and/or other decorative and functional materials can be applied to the carrier film to form a layered structure. Accordingly, one embodiment is further directed to a method of making a layered structure comprising: 1) applying a release coat to at least a portion of a carrier film; 2) applying electrically conductive material in a pattern to the carrier film after application of the release coat, wherein the electrically conductive material is applied on top of at least a portion of the release coat; 3) drying the electrically conductive material; 4) applying an adhesive over at least a portion of one or more of the electrically conductive material, release coat, or both; and 5) drying the adhesive. The electrically conductive material and adhesive may be dried after being applied such from 1 to 180 seconds, from 1 to 150 second, 1 to 120 seconds, 1 to 90 seconds, or any of the other drying times previously described. In addition, the layered structure can also include dielectric, decorative and/or functional materials applied over at least a portion of one or more of the release coat, electrically conductive material, adhesive, and carrier film. For example, a dielectric material and/or a decorative material can be applied on top of at least a portion of the release coat and/or the electrically conductive material. The dielectric, decorative, and functional materials may be applied in any desired pattern. The dielectric, decorative and functional materials may be dried independently or together (optionally with the other materials) after being applied, such as from 1 to 180 seconds, from 1 to 150 second, 1 to 120 seconds, 1 to 90 seconds, or any of the other drying times previously described.

The layered structure can be rolled for storage and/or shipping. For example, a layered structure can be formed by separately applying and optionally drying one or more of a release coat, electrically conductive material, adhesive, dielectric material, and decorative material onto a carrier film, and then the layered structure is coiled or recoiled into a roll. Accordingly, it may be desired that at least the outermost surface of the materials applied to the carrier film are tack free. The rolled tack free layered structure can later be unrolled and used in a heat stamping process to transfer electrically conductive materials to a substrate. By “tack free”, it is meant that the layered structure is dried to the touch and adheres to the substrate.

After applying the electrically conductive material (and optionally, other additional materials) onto the carrier film, the carrier film is contacted with a substrate. The substrate can be secured in place to prevent the substrate from moving and then the carrier film is contacted with the substrate. Heat and pressure are then applied to the substrate and carrier film, which includes the electrically conductive material and optionally any of the other materials described herein. For example, a layered structure may be contacted with a substrate that is secured in place or fixtured. Heat and pressure may then be applied to the layered structure and substrate. Heat and pressure can be applied with a hot stamping press, such as a rubber wheel hot stamping press. Heat and pressure are applied such that the electrically conductive material adheres to the substrate. One or more of an adhesive, dielectric material, release coat, and decorative material used with the carrier film can also be adhered to the substrate after applying heat and pressure. For example, an adhesive, dielectric material, and electrically conductive material can be adhered to the substrate after applying heat and pressure.

On embodiment deposits functionalized nanomaterials on flexible substrates. FIG. 4 shows an exemplary functionalized nano-material as amperometric biosensor for detecting hydrogen and the change in resistance of the sensor upon contact with hydrogen at room temperature. The resistance change of a semiconducting SWCNT with electrodeposited Pd particles upon exposure to hydrogen. Molecular hydrogen is split on the surface of a Pd particle into atomic hydrogen, which diffuses to the Pd/SWCNT interface. At this interface, a dipole layer is formed, which acts like a microscopic gate electrode that locally changes the charge-carrier concentration The recovery of the room-temperature-operated hydrogen sensor requires a supply of oxygen to remove the hydrogen atoms in the form of water.

Direct electron transfer can be done with various types of CNT electrodes for cytochrome c, horseradish peroxidase, myoglobin, as well as glucose oxidase where the redox-active center is deeply embedded within the protein. In some cases, aligned CNT arrays have been fabricated using self-assembly, followed by the covalent attachment of microperoxidase to the tube ends. A glucose sensor can be obtained by immobilizing glucose oxidase onto SWCNTs, for example. One embodiment includes single-walled carbon nanotubes (SWNT) applied as a coating to the working electrode. The SWNT can be, for example, a mixture of metallic and semiconducting SWNT. The SWNT provide an extremely large surface-to-volume ratio and have useful electrical properties. A sensor according to one embodiment operates by an electrochemical mechanism, whereby the presence of a particular analyte causes electron transfer in the electrochemical system, which can be identified and quantified by measuring a current through the sensor, which can be converted via amperometry to an output voltage. This feature of the present sensor renders it more accurate and reliable than other types of sensors that produce a change in electrical resistance of SWNT in the presence of an analyte. The electrode material can contain or consist of, for example and without limitation, gold, platinum, iridium, silver, silver/silver chloride, copper, aluminum, chromium, or other conductive metals or other conductive materials, or any combination thereof. In one embodiment, the SWNT are functionalized by a coating that includes an enzyme that catalyzes an electron transfer reaction and is specific for the selected analyte, such as glucose. Preferably the reaction is an oxidation reaction. For example, for the detection of glucose as the analyte, the enzyme glucose oxidase (GOx, EC 1.1.3.4) can be used, which specifically catalyzes the oxidation of β-D-glucose to hydrogen peroxide and D-glucono-6-lactone, which then hydrolyzes to gluconic acid. The enzyme can be a naturally occurring glucose oxidase enzyme which is isolated from a natural source (e.g. cells of Aspergillus niger), or it can be produced recombinantly in transformed or transfected host cells, such as bacterial cells, yeast or fungal cells, or mammalian cells. It can be glycosylated or non-glycosylated. The glucose oxidase enzyme used in the sensor can have a naturally occurring amino acid sequence, or it can have a mutant or engineered amino acid sequence. Different enzyme-functionalized SWNT can be combined in a multiplex sensor that takes advantage of the different sensitivities of each enzyme and their different resistance to inhibition induced by potentially interfering substances that might be encountered in a saliva sample. The sensor detects levels of glucose in saliva or another fluid by keeping track of the electrons passed through the glucose oxidase enzyme coated on the working electrode and measuring the resulting current, which is detected by an amperometry detection circuit and expressed as a change in output voltage. The sensing performance can be further improved by modifying the enzyme-coated electrode with various materials, including biomolecular or porous films or membranes. Such materials include, but are not limited to, carbon nanotubes, graphite, nanowires, gold nanoparticles (GNp), Pt nanoparticles, chitosan, bovine serum albumin (BSA), and Prussian Blue or other materials with similar properties. In one embodiment, the sensor of one embodiment detects glucose via an electrical signal resulting from the glucose oxidase reaction performed on functionalized SWNT connected to a detection circuit. It does not require any additional chemical reactions (e.g. peroxidase reaction) or optical detection means to detect the reaction products.

The system can be implemented on a flexible substrate with microneedles formed by impressing a bed of nails template onto the flexible substrate, onto which sweat can be captured and glucose and other important analytes can be captured. The system can be designed for single use (i.e., disposable) or for repeated use, with rinsing off, washing, or simple displacement of the sweat sample between readings. It can be used for real-time, noninvasive glucose monitoring for individuals at home and around clock. Through continuous or periodic glucose and/or analyte monitoring, additional temporal information can be obtained, such as trends, magnitude, duration, and frequency of certain glucose/analyte levels; this would allow tracking of data for better and more accurate assessment of a disease as well as the overall health condition of an individual. For example, the sensor system can activate an alarm for unusual or extreme glucose/analyte levels, decreasing the nursing workload when trying to maintain tight glycemic control. Such a system can also facilitate automatic feedback-controlled insulin delivery in an insulin delivery system, such as an artificial pancreas or insulin pump.

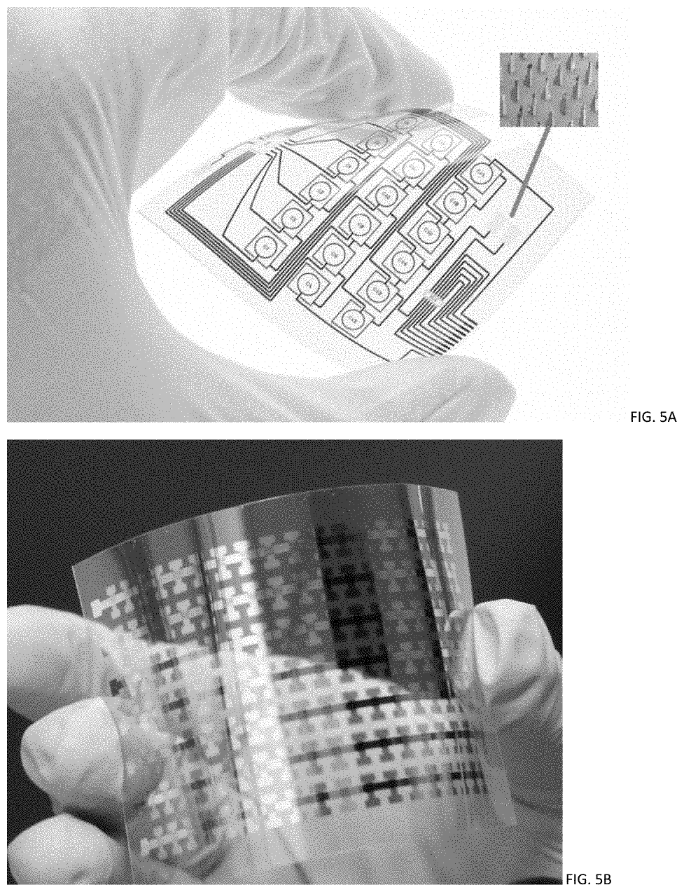

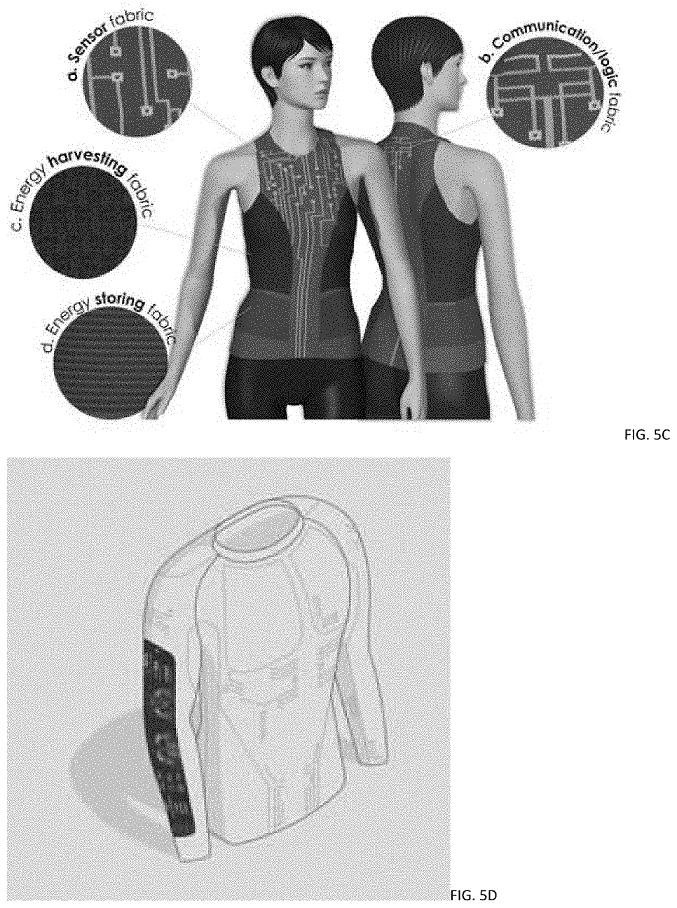

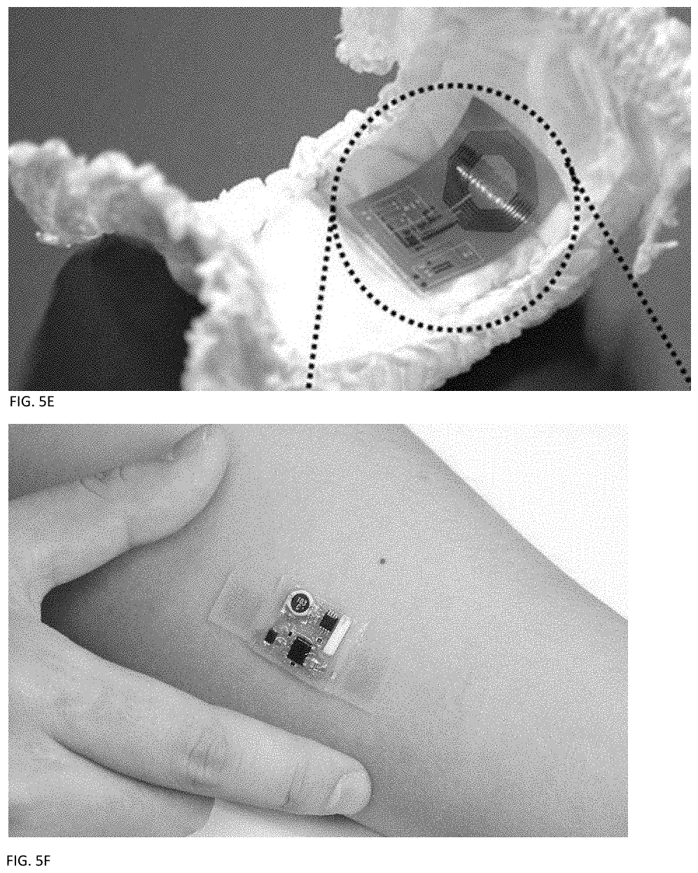

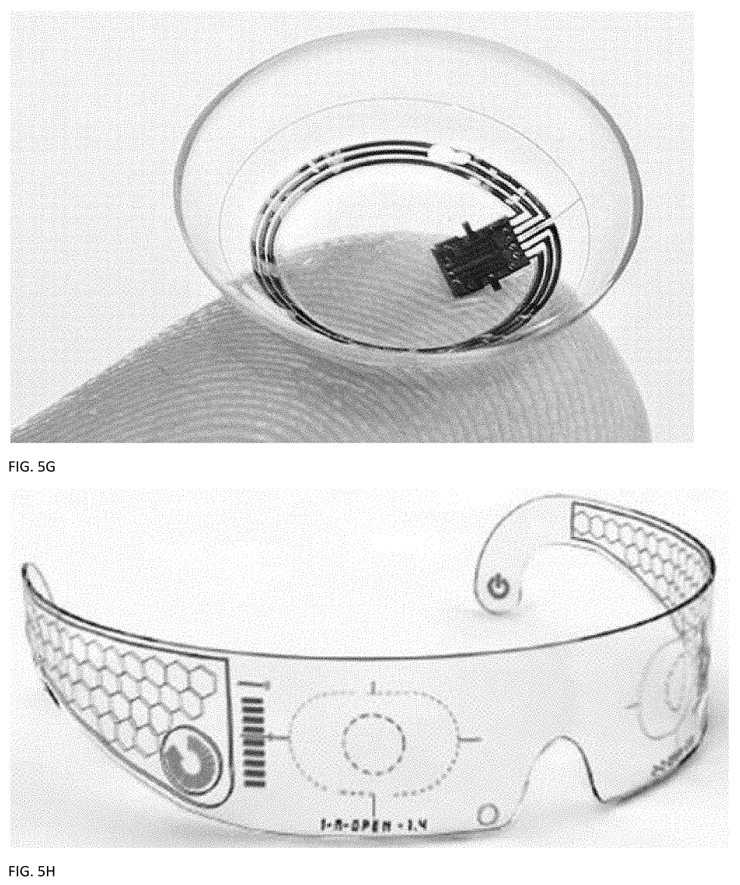

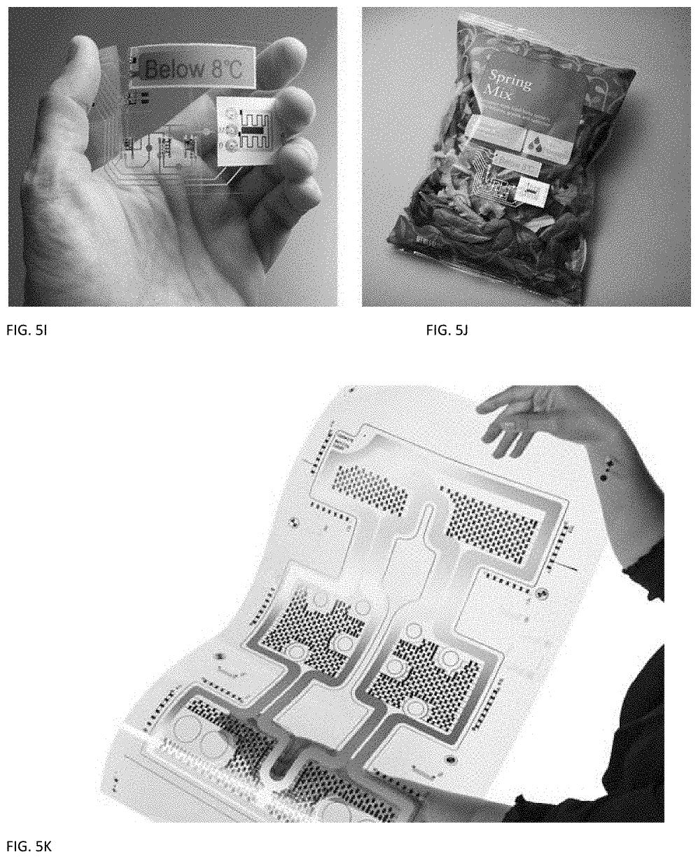

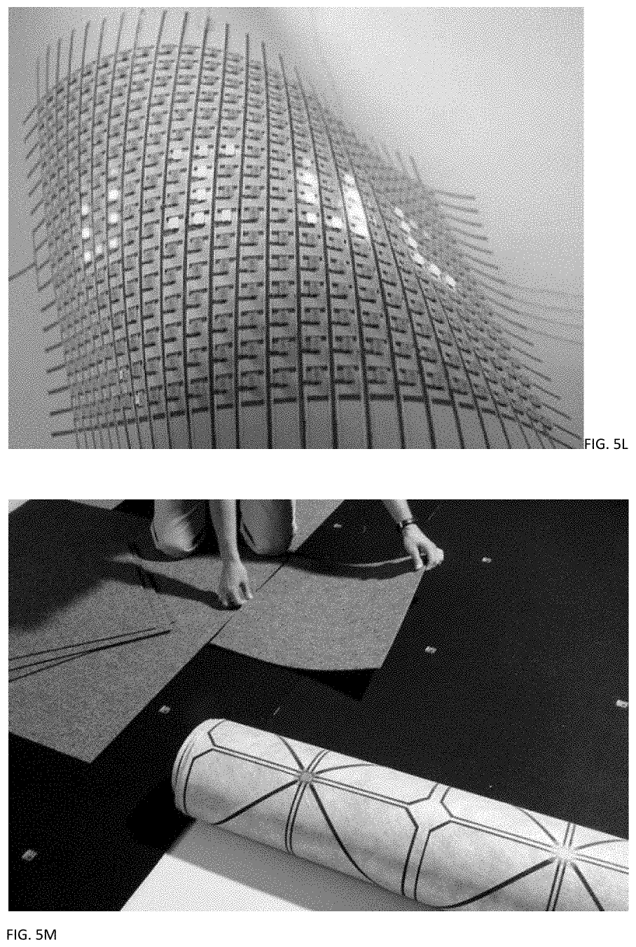

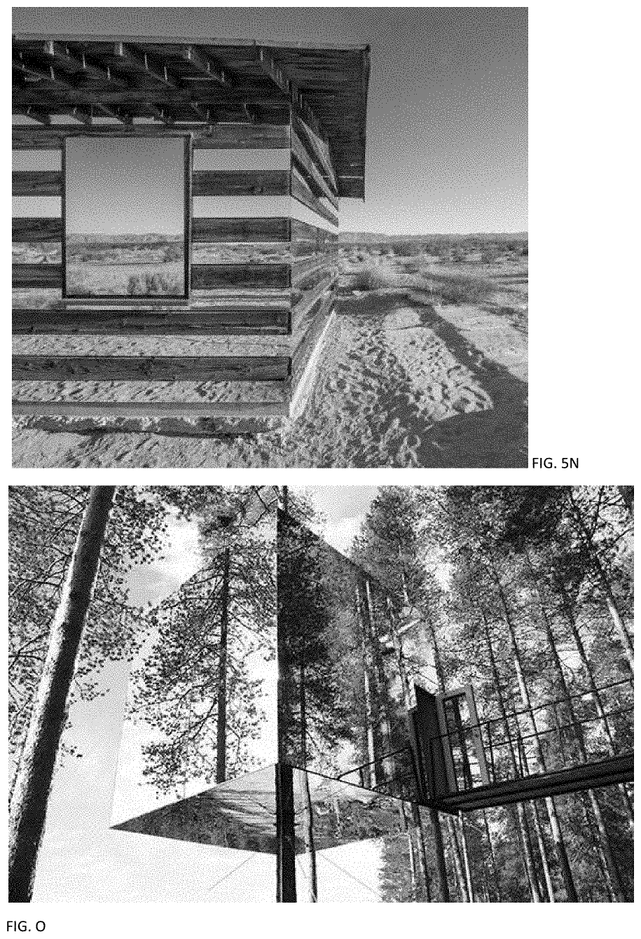

The flexible electronics can incorporate microneedles to extract deep subdermal fluids and/or to inject chemicals such as drugs into the blood stream upon detection of a trigger. For example, for diabetes, some microneedles extract sweats and/or glands secretion of glucose, and the glucose level is determined, and in a closed loop, drugs can be injected via another set of microneedles and suitable valves or seals that are opened on command. One such seal is opened by heaters on the microneedles to release the drugs. In one embodiment, a flexible skin patch can be made with functionalized macromolecules such as CNTs as sensors that detect humidity, glucose, pH, and temperature. The glucose sensor takes into account pH and temperature to improve the accuracy of the glucose measurements taken from sweat. If the skin patch senses high glucose levels, heaters trigger microneedles to dissolve a coating and release the drug metformin just below the skin surface. FIG. 5A shows an exemplary flexible printed electronic with microneedles thereon. The microneedles form an interface with the skin for detecting analyte or sugar levels in the person. In certain embodiments, a portion of the needles can inject medication in response to the detected levels in the person to form a close loop control system.

One embodiment provides a large skin patch with a sweat collection region to collect low quantity body fluid such as sweat. A flexible electronic pad can be printed for sweat collection with a channel layer, a container layer, and a vent layer. In some variations, the layers may be combined into a single layer and/or other layers may be added. The channel layer of the fixed volume device may contact the skin surface and direct sweat from the skin surface to an opening. On the skin surface, the sweat may be within or excreted from one or more sweat pores in contact with, or adjacent to, the channel layer. Typically, the container layer may be in fluid communication with an opening in the channel layer and may be in contact with the vent layer. The vent layer may be in contact with the container layer and may allow air to escape during sweat collection. The channel layer may have any number of channels to contact the skin for sweat collection. Upon contacting the skin surface, the channel layer may deform to contact as much skin as possible so that the channels may efficiently route sweat to the opening. The channel layer may have any suitable geometry or have any suitable dimensions. For example, the channel layer may have a thickness of about two hundred micrometers and the opening may have a diameter of less than about seven hundred micrometers. In some embodiments, the opening may have a diameter of greater than three hundred micrometers. The top side of the channel layer may define a bottom side of the container for holding the collected sweat. In these instances, the channel layer may or may not include one or more electrodes in contact with the container that is positioned to contact sweat within the container.

The container layer may be positioned on top of or extend from the channel layer, and may have the same size and shape as the channel layer or be of a different size and/or shape. The channel layer may include at least one opening opposite the container layer to draw the sweat from the skin surface. The container layer may include a feature that defines at least one side of the container. The feature may be a hole, a well, an indentation, an absorbent portion, or the like. The thickness of the container layer may be selected based on one or more factors such as the shape of the container, the volume of the container, or rigidity required for the container to maintain its shape when the channel layer is deformed. For example, the container layer may have a thickness of approximately 100, 200, 500, 700, or 1,000 micrometers. Like the channel layer, the container layer may also comprise one or more electrodes positioned to contact sweat within the container. The electrodes may be used in conjunction with a measurement device to, for example, determine when the container contains the fixed volume of sweat and/or to measure the sweat glucose level. The vent layer may be positioned on top of or extend from the container layer. In some variations, the functions performed by the vent layer may be performed by the container layer. The vent layer may reduce evaporation of sweat and/or provide an escape route for air within the container. In general, larger vents provide more fluid flow because the air can escape quickly but may allow more sweat to evaporate from the container.

To measure a glucose level from sweat, a system includes collecting a predetermined volume of sweat from skin using a skin patch and measuring the amount of glucose within the volume of sweat. The skin patch may be attached to any location on the body covered by skin. Typically, however, the skin patch is placed on a fingertip, hand, or forearm as these areas have a higher density of sweat glands, are easily accessible, and are currently used by diabetic patients for blood glucose testing. The skin patch may be a skin patch as described above or may be another skin patch that is configured to collect a predetermined volume of sweat. The predetermined volume of sweat may be less than about one-quarter microliter of sweat, about one-half microliter of sweat, about one microliter of sweat, about two microliters of sweat, about five microliters of sweat, about ten microliters of sweat, or any other suitable volume. Measuring the amount of glucose may comprise contacting the skin patch with a measurement device.

In some embodiments, the method also includes stimulating sweat production. Sweat production may be simulated chemically, e.g., by delivering pilocarpine to the skin surface. The pilocarpine may be wiped onto the skin surface prior to attachment of the skin patch. Sweat may also be stimulated by delivering heat or one or more other forms of energy to the surface of the skin. The patch itself may comprise a physical, chemical, or mechanical mechanism of inducing a local sweat response. For example, the patch may comprise pilocarpine, alone or with a permeation enhancer, or may be configured for iontophoretic delivery. Similarly, the patch may comprise one or more chemicals capable of inducing a local temperature increase, thereby initiating a local sweat response. In a like manner, the patch may also comprise one or more heaters for sufficient localized heating of the skin surface to induce an enhanced local sweat response.

The microneedles are formed above a substrate with a plurality of microneedle base parts projected from the substrate integrally. Then a microneedle tip part is formed on the top of each of the plurality of microneedle base parts, with in vivo solubility and biodegradability. A microneedle tip part intrusion recess is formed in the microneedle base part; and the microneedle tip part partially intrudes into the microneedle tip part intrusion recess. The plurality of microneedles is punctured into the skin so that the microneedle tip parts remain under the skin. The tip parts can administer an objective substance such as medication. The administration volume of the objective substance by the microneedle array as part of the flexible substrate 1 is controlled by the processor and varies depending on the EKG, heart rate, glucose level, K/Na level as detected by the electronics, and further based on the seriousness of symptom, the age, gender and weight of the administration subject, the administration period and intervals, and the type of active ingredients, and it is possible to select from the range that the administration volume as the medical active ingredients reaches the effective dose. Moreover, it is also possible to administrate the objective substance by the microneedle array on the flexible substrate 1, once a day, or divisionally twice or three times a day.

The applicable objective substances on the tip parts can include, as for hormones, luteinizing hormone-releasing hormone analog, insulin, faster-acting insulin analog, long-acting insulin analog, ultra-long-acting insulin analog, growth hormone, PEGylation human growth hormone analog, somatomedin C, natriuretic peptide, glucagon, follicle-stimulating hormone, GLP-1 analog, parathyroid hormone analog, and as for enzymes, t-PA, glucocerebrosidase, alpha-galactosidase A, alpha-L-iduronidase, acid alpha-glucosidase, iduronate-2-sulfatase, human N-acetylgalactosamine-4-sulfatase, urate oxidase, deoxyribonuclease, and as for blood coagulation/fibrinolysis-associated factors, blood coagulation factor VIII, blood coagulation factor VII, blood coagulation factor IX, thrombomodulin, and as for serum proteins, albumin, and as for interferons, interferon-alpha, interferon-beta, interferon-gamma, PEGylation interferon-alpha, and as for erythropoietins, erythropoietin, erythropoietin analog, PEGylation erythropoietin, and as for cytokines, G-CSF, G-CSF derivative, interleukin-2, bFGF, and as for antibodies, mouse anti-CD3 antibody, humanized anti-EGF receptor antibody, chimeric anti-CD20 antibody, humanized anti-RS virus antibody, chimeric anti-TNF-alpha antibody, chimeric anti-CD25 antibody, humanized anti-IL6 receptor antibody, calicheamicin binding humanized anti-CD33 antibody, humanized anti-VEGF antibody, MX-DTPA binding mouse anti-CD20 antibody, human anti-TNF-alpha antibody, chimeric anti-EGFR antibody, humanized anti-VEGF antibody fragment, humanized IgE antibody, human anti-complement-C5 antibody, human anti-EGFR antibody, human anti-IL12/IL23-p40 antibody, human anti-IL-1-beta antibody, human anti-RANKL antibody, humanized anti-CCR4 antibody, PEGylation humanized anti-TNF-alpha antibody Fab, and as for fusion proteins, soluble TNF receptor Fc fusion protein, CTLA4-modified Fc fusion protein, Fc-TPOR agonist peptide fusion protein, VEGFR-Fc fusion protein, and as for vaccines, tetanus toxoid, diphtheria toxoid, pertussis vaccine, inactivated polio vaccine, live polio vaccine, diphtheria-tetanus combined toxoid, pertussis diphtheria tetanus mixed vaccine, Haemophilus influenzae b (Hib) vaccine, hepatitis B vaccine, hepatitis A vaccine, influenza hemagglutinin vaccine, rabies vaccine, Japanese encephalitis vaccine, Weil's disease autumnalis combined vaccine, pneumococcus vaccine, human papilloma virus vaccine, mumps vaccine, varicella vaccine, rubella vaccine, measles vaccine, rotavirus vaccine, norovirus vaccine, RSV vaccine, BCG vaccine. Further, any substances having an effect of assisting activation of the medical agents or an effect of immune system adjustment, are also included in the medical agents of one embodiment, and for example, any adjuvants commonly used for manufacturing of vaccine formulations can be used. As for adjuvants, hardly water-soluble adjuvant, hydrophilic gel adjuvant or water-soluble adjuvant can be used. As for hardly water-soluble adjuvants, for example, retinoid such as retinoic acid, imiquimod, and imidazoquinolines such as Resquimod (R-848), 4-amino-α,α,2-dimethyl-1H-imidazo[4,5-c]quinoline-1-ethanol (R-842 (made by 3M Pharmaceuticals, etc.); Journal of Leukocyte Biology (1995) 58: see 365-372), 4-amino-α,α,2-trimethyl-1H-imidazo[4,5-c]quinoline-1-ethanol (S-27609 (made by 3M Pharmaceuticals, etc.); Journal of Leukocyte Biology (1995) 58: see 365-372), 4-amino-2-ethoxymethyl-α,α-dimethyl-1H-imidazo[4,5-c]quinoline-1-ethanol (S-28463 (made by 3M Pharmaceuticals, etc.); Antivirul Research (1995) 28: see 253-264), and Loxoribine, Bropirimine, oleic acid, liquid paraffin, and Freund's adjuvant are included. As for hydrophilic gel adjuvants, for example, aluminum hydroxide and aluminum phosphate are included. As for water-soluble adjuvants, for example, alpha-defensin, beta-defensin, cathelicidin, sodium alginate, poly[di(carboxylatophenoxy)phosphazene], Quil A, polyethylene imine are included. The preferable adjuvants are hydrophilic gel adjuvants and water-soluble adjuvants. As for hydrophilic gel adjuvants, aluminum hydroxide and aluminum phosphate are included.

In one embodiment, a system for manufacturing a flexible sensor device can include any combination of the thermal transfer printing system, plasma jet sprayer, inkjet printer, compositions, and/or other features described herein for inkjet printing onto a flexible substrate in order to prepare a flexible sensor device.

Methods such as physical vapor deposition, magnetron sputtering, plasma-enhanced chemical vapor deposition, hotolithography, and chemical vapor deposition may not be suitable for materials that cannot be processed under high vacuum due to outgassing issues. Screen printing is an inexpensive process for planar substrates.

Spin coating, blade coating and spray coating can be used. Spin coating is a method of coating which is widely used within lab scale OPV manufacturing and in general within the semiconductor industry, to dispense liquids in very uniform layers on planar substrates. In one embodiment, a Laurell lab scale spin coater can be used, where the substrate is mounted on a chuck that rotates the sample while dispensing the liquid onto the sample, first distributing the liquid and secondly applying a high rotational velocity to dispersing the liquid into a uniform film thickness. Slot-die coating is a non-contact large-area processing method for the deposition of homogeneous wet films with high cross-directional uniformity. The slot-die coating head is made from stainless steel and contains an ink distribution chamber, feed slot, and an up- and downstream lip. An internal mask (shim) defines the feed slot width and allows stripe coating.

Inkjet and aerosol printing can be used but may need post-deposition thermal treatment for the formation of a uniform film and removing organic contaminants. Spray coating is widely known as an (industrial) method for car body painting and from graffiti artists using spray cans. The functional fluid or ink is atomized at the nozzle of the spray head, which generates a continuous flow of droplets. Pneumatic-based systems use a stream of pressurized air or gas (e.g. helium, nitrogen or argon) that breaks up the liquid into droplets at the nozzle. Parameters for the atomization process are surface tension, viscosity, fluid density, gas flow properties, and nozzle design. The quality of the coated layer is defined by the wetting behavior, surface properties, working distance, coating speed, droplet sizes, and the amount of sprayed layers. Besides the fluid-surface interaction the kinetic impact of the droplets influence the spreading of the droplets. An airbrush gun can be used, but other spray generation methods can be used such as ultrasonication with directed carrier gases, or electro-spraying.

In one embodiment, a method of manufacturing a flexible sensor device can include plasma spraying (plasma jetting or simply jetting) a nanosensor-containing composition onto a flexible substrate so as to deposit and retain one or more of nanosensors in a first predetermined pattern of a first macrosensor on the flexible substrate. The flexible substrate that has jet-printed nanosensors can be configured to have a desired degree of elongation, contraction and distortion while retaining sensing functions of the nanosensors. Such configuration can be achieved by the flexible substrate having such flexibility. Also, the jetted composition can include components, such as binders, elastomers, polymers, or the like, that provide post printing flexibility. In another embodiment, the method of manufacture can include jetting a second nanosensor-containing composition onto the flexible substrate. The second nanosensor-containing composition can include nanosensors that are different from the other nanosensors. The nanosensors can be configured to detect different target substances. Alternatively, the nanosensors can be a different type that detects the same target substance. In yet another embodiment, manufacturing can include jetting a conducting polymer-containing composition onto the flexible substrate so as to form a sensor circuit that is operably coupled with at least one jet printed nanosensor. The sensor circuit can include circuit components formed from the conducting polymer. The jetting of the conducting polymer-containing composition can also include the jetting of components that form a conducting polymer, such as, monomers, polymerizers, dopants, reactants, binders, polymers, conductive components, metallic components, and the like that can form a conducting polymer in a circuit configuration. Thus, the printing of a conducting polymer can be performed by printing components that combine to form a conducting polymer on the substrate. In one embodiment, manufacturing can include jetting a nanowire complex-containing composition onto the flexible substrate so as to form a sensor circuit that is operably coupled with at least one jet printed nanosensor. The sensor circuit can include circuit components formed from the nanowire. The jetting of the nanowire-containing composition can also include the jetting of components that form a nanowire, such as, semiconductor materials, monomers, polymerizers, dopants, reactants, binders, polymers, and the like that can form a nanowire in a circuit configuration. Thus, the printing of a nanowire polymer can be performed by printing components that combine to form a conducting polymer on the substrate.

An atmospheric-pressure plasma jet deposition can be done using a dielectric barrier discharge and can provide high-throughput processing and can coat three-dimensional objects. The presence of a dielectric material between the electrodes at the nozzle reduces the current filament, resulting in lowtemperature deposition suitable for low glass transition temperature materials.

The plasma jet printer consists of a quartz nozzle containing two copper electrodes and connected to a high-voltage (1 to 15 kV AC) power supply. A fixed aerosol flow is provided with plasma turned-off. A dielectric barrier discharge of helium is generated upon applying a potential between the electrodes. A container with a colloid of the functionalized nanomaterial to be deposited is placed on a nebulizer that generates an aerosol of the colloid, and the aerosol is carried by a helium carrier gas into the quartz tube containing the plasma. The deposition takes place at room temperature on the substrate placed closely to the nozzle. The sprayer jet does not need a vacuum pump and vacuum chamber as the process takes place at atmospheric pressure to reduce damage to the functionalized multiwalled carbon nanotubes.

In one embodiment, manufacturing can include plasmajetting a conducting polymer-containing composition and a nanowire complex-containing composition onto the flexible substrate so as to form a sensor circuit that is operably coupled with at least one jetted nanosensor. The conducting polymer and nanowire complex can cooperate to form the sensor circuit. The conducting polymer-containing composition can be retained in a separate reservoir from the nanowire complex-containing composition. As before, the formation of the sensor circuit can be performed by printing pre-conducting polymer components and/or pre-nanowire components that form conducting polymers and/or nanowires on the substrate so as to form the sensor circuit.

In one embodiment, the flexible substrate can be incorporated into a wearable garment. Wearable garments that include sensors can be used for sensing biometric data as well as sensing target substances as described herein. In some instances, the biometric data can be obtained from detecting target substances. As such, the method of manufacture can include configuring the flexible substrate having the jet-printed nanosensors with sufficient flexibility for being a component of a wearable garment such that the macrosensor is capable of sensing biometric data of a subject wearing the wearable garment. The sensors can detect a chemical that is provided from a subject wearing the garment, and the detection of the chemical or determination of the amount or concentration of the chemical in or on the subject can provide biometric data. Biometric data can then be used for health purposes and/or determine the health state of the subject.

In one embodiment, a nanosensor-containing composition can be jetted onto the flexible substrate so as to deposit and retain one or more of nanosensors in at least a second predetermined pattern of at least a second macrosensor on the flexible substrate. The first and second macrosensors can be separated by cutting the flexible substrate. Alternatively, the first macrosensor can be placed onto the second macrosensor and the flexible substrate can be adhered together to form a pouch having both macrosensors. Also, this can include operably coupling a second macrosensor with the first macrosensor.

The method of manufacture can include placing a second flexible substrate onto the flexible substrate having the jet-printed nanosensors, and bonding the second flexible substrate to the flexible substrate having the jet-printed nanosensors. This can be used to prepare the sensor devices as described herein. Also, the flexible substrate can be folded onto itself and bonded to form a container as described herein.

Accordingly, a method of preparing a flexible sensor device by jet printing can include jetting a sensor-containing composition onto a flexible substrate so as to deposit and retain one or more sensors in a first predetermined pattern of a first sensor (e.g., macrosensor) on the flexible substrate. The jet printed sensor can have the flexibility, elongation, contraction, and/or distortion properties as described herein. The flexible substrate having the jet-printed sensors is configured to have a desired degree of elongation, contraction, and distortion while retaining sensing functions of the sensors. Also, the jetted composition can include components, such as binders, elastomers, polymers, or the like, that provide post printing flexibility.

In one embodiment, the method of manufacturing a flexible sensor device can also include any one or combination of the following: jetting a second sensor-containing composition onto the flexible substrate; jetting a conducting polymer-containing composition onto the flexible substrate so as to form a sensor circuit that is operably coupled with at least one jet printed sensor; jetting a nanowire complex-containing composition onto the flexible substrate so as to form a sensor circuit that is operably coupled with at least one jet printed sensor; jetting a nanowire complex-containing composition onto the flexible substrate so as to form a sensor circuit that is operably coupled with at least one jetted sensor and the jetted nanowire complex containing sensor circuit, wherein the conducting polymer-containing composition is retained from a separate reservoir from the nanowire complex-containing composition; or jetting a sensor-containing composition onto the flexible substrate so as to deposit and retain a plurality of sensors in at least a second predetermined pattern of at least a second macrosensor on the flexible substrate; or operably coupling a second macrosensor with the first sensor (e.g., first macrosensor). Such manufacturing steps can be performed as described herein or known in the art. The printed sensors can be individual sensor or any number of sensors together so as to form a macrosensor. Macrosensors are considered to be a sensor formed of sensors and/or nanosensors.

In one embodiment, a method of manufacturing a flexible sensor device having one or more sensor circuits by jet printing. The jet printing method can include jetting at least one composition having components for forming a sensor circuit onto a flexible substrate so as to form and retain at least one sensor circuit on the flexible substrate in a predetermined pattern. The sensor circuit can be configured for sensing an interaction with a target substance. The flexible substrate having the jet-printed sensor circuit can be configured to have a desired degree of elongation, contraction, and distortion while retaining sensing functions of the sensor circuit.

In one embodiment, the method of manufacture can also include any of the following: preparing the at least one composition having components for forming the sensor circuit to have a conducting polymer-containing composition configured for being jetted onto the flexible substrate; preparing the at least one composition having components for forming the sensor circuit to include a nanowire complex-containing composition configured for being jetted onto the flexible substrate; jetting a conducting polymer-containing composition onto the flexible substrate so as to form the sensor circuit; jetting a nanowire complex-containing composition onto the flexible substrate so as to form the sensor circuit; jetting a conducting polymer-containing composition and a nanowire complex-containing composition onto the flexible substrate so as to form the sensor circuit; jetting a nanosensor-containing composition onto the flexible substrate so as to deposit and retain a plurality of nanosensors in a first predetermined pattern of a first macrosensor on the flexible substrate, said flexible substrate having the jet-printed nanosensors being configured to have a desired degree of elongation, contraction and distortion while retaining sensing functions of the nanosensors, the first macrosensor being operably coupled with the at least one sensing circuit and being configured to interact with a target substance; or configuring the flexible substrate having the jet-printed nanosensors with sufficient flexibility for being a component of a wearable garment such that the macrosensor is capable of sensing biometric data of a subject wearing the wearable garment. Also, the method can include placing a second flexible substrate onto the flexible substrate having the jet-printed sensor circuit, and bonding the second flexible substrate to the flexible substrate having the jet-printed sensor circuit. Such manufacturing steps can be performed as described herein or known in the art.

A chain of wells and channels on substrates can be formed as microfluidic cassettes or devices that can be used to effect a number of manipulations on a sample to ultimately result in target analyte detection or quantification. These manipulations can include cell handling (cell concentration, cell lysis, cell removal, cell separation, etc.), separation of the desired target analyte from other sample components, chemical or enzymatic reactions on the target analyte, detection of the target analyte, etc. The devices can include one or more wells for sample manipulation, waste or reagents; channels to and between these wells, including channels containing electrophoretic separation matrices; valves to control fluid movement; on-chip pumps such as electroosmotic, electrohydrodynamic, or electrokinetic pumps; and detection systems comprising electrodes, as is more fully described below. The devices of can be configured to manipulate one or multiple samples or analytes.

The microfluidic devices are used to detect target analytes in samples. By “target analyte” or “analyte” or grammatical equivalents herein is meant any molecule, compound or particle to be detected. As outlined below, target analytes preferably bind to binding ligands, as is more fully described above. As will be appreciated by those in the art, a large number of analytes may be detected using the present methods; basically, any target analyte for which a binding ligand, described herein, may be made may be detected using the methods of the invention.

Suitable analytes include organic and inorganic molecules, including biomolecules. In one embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (TM) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This is particularly advantageous in the systems of the system, as a reduced salt hybridization solution has a lower Faradaic current than a physiological salt solution (in the range of 150 mM).

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as nucleosides.

In one embodiment, the system provides methods of detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 basepairs, with fragments of roughly 500 basepairs being preferred in some embodiments. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

The probes (including primers) are made to hybridize to target sequences to determine the presence or absence of the target sequence in a sample. Generally speaking, this term will be understood by those skilled in the art.

The target sequence may also be comprised of different target domains; for example, in “sandwich” type assays as outlined below, a first target domain of the sample target sequence may hybridize to a capture probe or a portion of capture extender probe, a second target domain may hybridize to a portion of an amplifier probe, a label probe, or a different capture or capture extender probe, etc. In addition, the target domains may be adjacent (i.e. contiguous) or separated. For example, when ligation chain reaction (LCR) techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below.

In one embodiment, the target analyte is a protein. As will be appreciated by those in the art, there are a large number of possible proteinaceous target analytes that may be detected using the system. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In one embodiment, the amino acids are in the (S) or L-configuration. As discussed below, when the protein is used as a binding ligand, it may be desirable to utilize protein analogs to retard degradation by sample contaminants.

Suitable protein target analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g. respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus); hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (PA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-a and TGF-(3), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (4) other proteins (including a-fetoprotein, carcinoembryonic antigen CEA, cancer markers, etc.).

In addition, any of the biomolecules for which antibodies may be detected may be detected directly as well; that is, detection of virus or bacterial cells, therapeutic and abused drugs, etc., may be done directly.

Suitable target analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

Suitable target analytes include metal ions, particularly heavy and/or toxic metals, including but not limited to, aluminum, arsenic, cadmium, selenium, cobalt, copper, chromium, lead, silver and nickel.

These target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc.

At least one channel or flow channel allows the flow of sample from the sample inlet port to the other components or modules of the system. The collection of channels and wells is sometimes referred to in the art as a “mesoscale flow system”. The flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used.

In addition to the flow channel system, the microfluidic devices are configured to include one or more of a variety of components, herein referred to as “modules”, that will be present on any given device depending on its use. These modules include, but are not limited to: sample inlet ports; sample introduction or collection modules; cell handling modules (for example, for cell lysis, cell removal, cell concentration, cell separation or capture, cell growth, etc.); separation modules, for example, for electrophoresis, dielectrophoresis, gel filtration, ion exchange/affinity chromatography (capture and release) etc.; reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), and nucleic acid sequence based amplification (NASBA)), chemical, physical or enzymatic cleavage or alteration of the target analyte, or chemical modification of the target; fluid pumps; fluid valves; thermal modules for heating and cooling; storage modules for assay reagents; mixing chambers; and detection modules.

In one embodiment, the devices include a cell handling module. This is of particular use when the sample comprises cells that either contain the target analyte or that must be removed in order to detect the target analyte. Thus, for example, the detection of particular antibodies in blood can require the removal of the blood cells for efficient analysis, or the cells (and/or nucleus) must be lysed prior to detection. In this context, “cells” include eukaryotic and prokaryotic cells, and viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, cell concentration modules, and cell separation or capture modules. In addition, as for all the modules of the invention, the cell handling module is in fluid communication via a flow channel with at least one other module of the invention.

In one embodiment, the cell handling module includes a cell lysis module. The cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In one embodiment, the cell lysis module comprises a cell lysing agent, such as guanidium chloride, chaotropic salts, enzymes such as lysozymes, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample. The cell lysis module may also include, either internally or externally, a filtering module for the removal of cellular debris as needed. This filter may be microfabricated between the cell lysis module and the subsequent module to enable the removal of the lysed cell membrane and other cellular debris components.

In one embodiment, the cell handling module includes a cell separation or capture module. This embodiment utilizes a cell capture region comprising binding sites capable of reversibly binding a cell surface molecule to enable the selective isolation (or removal) of a particular type of cell from the sample population, for example, white blood cells for the analysis of chromosomal nucleic acid, or subsets of white blood cells. These binding moieties may be immobilized either on the surface of the module or on a particle trapped within the module (i.e. a bead) by physical absorption or by covalent attachment. Suitable binding moieties will depend on the cell type to be isolated or removed, and generally includes antibodies and other binding ligands, such as ligands for cell surface receptors, etc.

Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

Alternatively, a cellular “sieve” can be used to separate cells on the basis of size. This can be done in a variety of ways, including protrusions from the surface that allow size exclusion, a series of narrowing channels, a weir, or a diafiltration type setup.

In one embodiment, the cell handling module includes a cell removal module. This may be used when the sample contains cells that are not required in the assay or are undesirable. Generally, cell removal will be done on the basis of size exclusion as for “sieving”, above, with channels exiting the cell handling module that are too small for the cells.

In one embodiment, the cell handling module includes a cell concentration module. As will be appreciated by those in the art, this is done using “sieving” methods, for example to concentrate the cells from a large volume of sample fluid prior to lysis.

In one embodiment, the devices include a separation module. Separation in this context means that at least one component of the sample is separated from other components of the sample. This can comprise the separation or isolation of the target analyte, or the removal of contaminants that interfere with the analysis of the target analyte, depending on the assay.

In one embodiment, the separation module includes an electrophoresis module where molecules are primarily separated by different electrophoretic mobilities caused by their different molecular size, shape and/or charge. Microcapillary tubes are used in microcapillary gel electrophoresis (high performance capillary electrophoresis (HPCE)). One advantage of HPCE is that the heat resulting from the applied electric field is efficiently disappated due to the high surface area, thus allowing fast separation. The electrophoresis module serves to separate sample components by the application of an electric field, with the movement of the sample components being due either to their charge or, depending on the surface chemistry of the channel, bulk fluid flow as a result of electroosmotic flow (EOF).

As will be appreciated by those in the art, the electrophoresis module can take on a variety of forms, and generally comprises an electrophoretic channel and associated electrodes to apply an electric field to the electrophoretic channel. Waste fluid outlets and reservoirs are present as required. Electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose.

In one embodiment, the devices include a reaction module. This can include physical, chemical or biological alteration of one or more sample components. Alternatively, it may include a reaction module wherein the target analyte alters a second moiety that can then be detected; for example, if the target analyte is an enzyme, the reaction chamber may comprise an enzyme substrate that upon modification by the target analyte, can then be detected. In this embodiment, the reaction module may contain the necessary reagents, or they may be stored in a storage module and pumped as outlined herein to the reaction module as needed. In one embodiment, the reaction module includes a chamber for the chemical modification of all or part of the sample. For example, chemical cleavage of sample components (CNBr cleavage of proteins, etc.) or chemical cross-linking can be done. In one embodiment, the reaction module includes a chamber for the biological alteration of all or part of the sample. For example, enzymatic processes including nucleic acid amplification, hydrolysis of sample components or the hydrolysis of substrates by a target enzyme, the addition or removal of detectable labels, the addition or removal of phosphate groups, etc.

In one embodiment, the target analyte is a nucleic acid and the biological reaction chamber allows amplification of the target nucleic acid. Suitable amplification techniques include, both target amplification and probe amplification, including, but not limited to, polymerase chain reaction (PCR), ligase chain reaction (LCR), strand displacement amplification (SDA), self-sustained sequence replication (3SR), QB replicase amplification (QBR), repair chain reaction (RCR), cycling probe technology or reaction (CPT or CPR), and nucleic acid sequence based amplification (NASBA). In most cases, double stranded target nucleic acids are denatured to render them single stranded so as to permit hybridization of the primers and other probes of the invention. One embodiment utilizes a thermal step, generally by raising the temperature of the reaction to about 95° C., although pH changes and other techniques such as the use of extra probes or nucleic acid binding proteins may also be used. A probe nucleic acid (also referred to herein as a primer nucleic acid) is then contacted to the target sequence to form a hybridization complex. By “primer nucleic acid” herein is meant a probe nucleic acid that will hybridize to some portion, i.e. a domain, of the target sequence. Probes of the system are designed to be complementary to a target sequence (either the target sequence of the sample or to other probe sequences, as is described below), such that hybridization of the target sequence and the probes of the system occurs. As outlined below, this complementarity need not be perfect; there may be any number of base pair mismatches which will interfere with hybridization between the target sequence and the single stranded nucleic acids of the system. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

Once the hybridization complex between the primer and the target sequence has been formed, an enzyme, sometimes termed an “amplification enzyme”, is used to modify the primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides are added to the primer to extend its length. Once the enzyme has modified the primer to form a modified primer, the hybridization complex is disassociated. After a suitable time or amplification, the modified primer is moved to a detection module and incorporated into an assay complex, as is more fully outlined below. The assay complex is covalently attached to an electrode, and comprises at least one electron transfer moiety (ETM), described below. Electron transfer between the ETM and the electrode is then detected to indicate the presence or absence of the original target sequence, as described below.

In one embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA).

In one embodiment, the target amplification technique is PCR. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostabile polymerase such as Taq I polymerase and thermal cycling.

In one embodiment, the target amplification technique is Strand displacement amplification (SDA) where a single stranded target nucleic acid, usually a DNA target sequence, is contacted with an SDA primer. An “SDA primer” generally has a length of 25-100 nucleotides, with SDA primers of approximately 35 nucleotides being preferred. An SDA primer is substantially complementary to a region at the 3′ end of the target sequence, and the primer has a sequence at its 5′ end (outside of the region that is complementary to the target) that is a recognition sequence for a restriction endonuclease, sometimes referred to herein as a “nicking enzyme” or a “nicking endonuclease”, as outlined below. The SDA primer then hybridizes to the target sequence. The SDA reaction mixture also contains a polymerase (an “SDA polymerase”, as outlined below) and a mixture of all four deoxynucleoside-triphosphates (also called deoxynucleotides or dNTPs, i.e. dATP, dTTP, dCTP and dGTP), at least one species of which is a substituted or modified dNTP; thus, the SDA primer is modified, i.e. extended, to form a modified primer, sometimes referred to herein as a “newly synthesized strand”. The substituted dNTP is modified such that it will inhibit cleavage in the strand containing the substituted dNTP but will not inhibit cleavage on the other strand. Examples of suitable substituted dNTPs include, but are not limited, 2′deoxyadenosine 5′-O-(1-thiotriphosphate), 5-methyldeoxycytidine 5′-triphosphate, 2′-deoxyuridine 5′-triphosphate, adn 7-deaza-2′-deoxyguanosine 5′-triphosphate. In addition, the substitution of the dNTP may occur after incorporation into a newly synthesized strand; for example, a methylase may be used to add methyl groups to the synthesized strand. In addition, if all the nucleotides are substituted, the polymerase may have 5′3′ exonuclease activity. However, if less than all the nucleotides are substituted, the polymerase preferably lacks 5′3′ exonuclease activity. Once nicked, a polymerase (an “SDA polymerase”) is used to extend the newly nicked strand, 5′3′, thereby creating another newly synthesized strand. The polymerase chosen should be able to intiate 5′3′ polymerization at a nick site, should also displace the polymerized strand downstream from the nick, and should lack 5′3′ exonuclease activity (this may be additionally accomplished by the addition of a blocking agent). Thus, suitable polymerases in SDA include, but are not limited to, the Klenow fragment of DNA polymerase I, SEQUENASE 1.0 and SEQUENASE 2.0 (U.S. Biochemical), T5 DNA polymerase and Phi29 DNA polymerase. Accordingly, the SDA reaction requires, in no particular order, an SDA primer, an SDA polymerase, a nicking endonuclease, and dNTPs, at least one species of which is modified.

In one embodiment, the target amplification technique is nucleic acid sequence based amplification (NASBA). A single stranded target nucleic acid, usually an RNA target sequence (sometimes referred to herein as “the first target sequence” or “the first template”), is contacted with a first NASBA primer. A “NASBA primer” generally has a length of 25100 nucleotides, with NASBA primers of approximately 50-75 nucleotides being preferred. The first NASBA primer is preferably a DNA primer that has at its 3′ end a sequence that is substantially complementary to the Tend of the first template. The first NASBA primer has an RNA polymerase promoter at its Fend. The first NASBA primer is then hybridized to the first template to form a first hybridization complex. The NASBA reaction mixture also includes a reverse transcriptase enzyme (an “NASBA reverse transcriptase”) and a mixture of the four dNTPs, such that the first NASBA primer is modified, i.e. extended, to form a modified first primer, comprising a hybridization complex of RNA (the first template) and DNA (the newly synthesized strand).

In one embodiment, the amplification technique is signal amplification. Signal amplification involves the use of limited number of target molecules as templates to either generate multiple signalling probes or allow the use of multiple signalling probes. Signal amplification strategies include LCR, CPT, and the use of amplification probes in sandwich assays.

In one embodiment, the devices include at least one fluid pump. Pumps generally fall into two categories: “on chip” and “off chip”; that is, the pumps (generally electrode based pumps) can be contained within the device itself, or they can be contained on an apparatus into which the device fits, such that alignment occurs of the required flow channels to allow pumping of fluids. In one embodiment, the pumps are contained on the device itself. These pumps are generally electrode based pumps; that is, the application of electric fields can be used to move both charged particles and bulk solvent, depending on the composition of the sample and of the device. Suitable on chip pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel to result in the pumping of the fluids comprising the sample components. As is described in the art, the configurations for each of these electrode based pumps are slighly different; for example, the effectiveness of an EHD pump depends on the spacing between the two electrodes, with the closer together they are, the smaller the voltage required to be applied to effect fluid flow. Alternatively, for EO pumps, the sampling between the electrodes should be larger, with up to one-half the length of the channel in which fluids are being moved, since the electrode are only involved in applying force, and not, as in EHD, in creating charges on which the force will act. In one embodiment, an electroosmotic pump is used. Electroosmosis (EO) is based on the fact that the surface of many solids, including quartz, glass and others, become variously charged, negatively or positively, in the presence of ionic materials. The charged surfaces will attract oppositely charged counterions in aqueous solutions. Applying a voltage results in a migration of the counterions to the oppositely changed electrode, and moves the bulk of the fluid as well. The volume flow rate is proportional to the current, and the volume flow generated in the fluid is also proportional to the applied voltage. Electroosmostic flow is useful for liquids having some conductivity is and generally not applicable for non-polar solvents. In one embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents. In one embodiment, a micromechanical pump is used, either on- or off chip, as is known in the art.

In one embodiment, an “off-chip” pump is used. For example, the devices may fit into an apparatus or appliance that has a nesting site for holding the device, that can register the ports (i.e. sample inlet ports, fluid inlet ports, and waste outlet ports) and electrode leads. The apparatus can including pumps that can apply the sample to the device; for example, can force cell containing samples into cell lysis modules containing protrusions, to cause cell lysis upon application of sufficient flow pressure. Such pumps are well known in the art.

In one embodiment, the devices include at least one fluid valve that can control the flow of fluid into or out of a module of the device, or divert the flow into one or more channels. In one embodiment, the devices include sealing ports, to allow the introduction of fluids, including samples, into any of the modules of the invention, with subsequent closure of the port to avoid the loss of the sample. In one embodiment, the devices include at least one storage modules for assay reagents. These are connected to other modules of the system using flow channels and may comprise wells or chambers, or extended flow channels. They may contain any number of reagents, buffers, enzymes, electronic mediators, salts, etc., including freeze dried reagents. In one embodiment, the devices include a mixing module; again, as for storage modules, these may be extended flow channels (particularly useful for timed mixing), wells or chambers. Particularly in the case of extended flow channels, there may be protrusions on the side of the channel to cause mixing.

One embodiment uses detection electrode comprises a self-assembled monolayer (SAM) comprising conductive oligomers. By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer. The SAM may comprise conductive oligomers alone, or a mixture of conductive oligomers and insulators. As outlined herein, the efficiency of target analyte binding (for example, oligonucleotide hybridization) may increase when the analyte is at a distance from the electrode. Similarly, nonspecific binding of biomolecules, including the target analytes, to an electrode is generally reduced when a monolayer is present. Thus, a monolayer facilitates the maintenance of the analyte away from the electrode surface. In addition, a monolayer serves to keep charged species away from the surface of the electrode. Thus, this layer helps to prevent electrical contact between the electrodes and the ETMs, or between the electrode and charged species within the solvent. Such contact can result in a direct “short circuit” or an indirect short circuit via charged species which may be present in the sample. Accordingly, the monolayer is preferably tightly packed in a uniform layer on the electrode surface, such that a minimum of “holes” exist. The monolayer thus serves as a physical barrier to block solvent accesibility to the electrode.

In one embodiment, electronic detection is used, including amperommetry, voltammetry, capacitance, and impedence. Suitable techniques include, but are not limited to, electrogravimetry; coulometry (including controlled potential coulometry and constant current coulometry); voltametry (cyclic voltametry, pulse voltametry, (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

In one embodiment, monitoring electron transfer is via amperometric detection. This method of detection involves applying a potential (as compared to a separate reference electrode) between the nucleic acid-conjugated electrode and a reference (counter) electrode in the sample containing target genes of interest. Electron transfer of differing efficiencies is induced in samples in the presence or absence of target nucleic acid; that is, the presence or absence of the target nucleic acid, and thus the label probe, can result in different currents.

The device for measuring electron transfer amperometrically involves sensitive current detection and includes a means of controlling the voltage potential, usually a potentiostat. This voltage is optimized with reference to the potential of the electron donating complex on the label probe. Possible electron donating complexes include those previously mentioned with complexes of iron, osmium, platinum, cobalt, rhenium and ruthenium being preferred and complexes of iron being most preferred.

Alternatively, the compositions are useful to detect successful gene amplification in PCR, thus allowing successful PCR reactions to be an indication of the presence or absence of a target sequence. PCR may be used in this manner in several ways. For example, in one embodiment, the PCR reaction is done as is known in the art, and then added to a composition comprising the target nucleic acid with a ETM, covalently attached to an electrode via a conductive oligomer with subsequent detection of the target sequence. Alternatively, PCR is done using nucleotides labelled with a ETM, either in the presence of, or with subsequent addition to, an electrode with a conductive oligomer and a target nucleic acid. Binding of the PCR product containing ETMs to the electrode composition will allow detection via electron transfer. Finally, the nucleic acid attached to the electrode via a conductive polymer may be one PCR primer, with addition of a second primer labelled with an ETM. Elongation results in double stranded nucleic acid with a ETM and electrode covalently attached. In this way, the system is used for PCR detection of target sequences.

In one embodiment, the arrays are used for mRNA detection. One embodiment utilizes either capture probes or capture extender probes that hybridize close to the 3′ polyadenylation tail of the mRNAs. This allows the use of one species of target binding probe for detection, i.e. the probe contains a poly-T portion that will bind to the poly-A tail of the mRNA target. Generally, the probe will contain a second portion, preferably non-poly-T, that will bind to the detection probe (or other probe). This allows one target-binding probe to be made, and thus decreases the amount of different probe synthesis that is done.

In one embodiment, the use of restriction enzymes and ligation methods allows the creation of “universal” arrays. In this embodiment, monolayers comprising capture probes that comprise restriction endonuclease ends. By utilizing complementary portions of nucleic acid, while leaving “sticky ends”, an array comprising any number of restriction endonuclease sites is made. Treating a target sample with one or more of these restriction endonucleases allows the targets to bind to the array. This can be done without knowing the sequence of the target. The target sequences can be ligated, as desired, using standard methods such as ligases, and the target sequence detected, using either standard labels or the methods of the invention.

As outlined herein, the devices can be used in combination with apparatus for delivering and receiving fluids to and from the devices. The apparatus can include a “nesting site” for placement of the device(s) to hold them in place and for registering inlet and outlet ports, if present. The apparatus may also include pumps (“off chip pumps”), and means for viewing the contents of the devices, including microscopes, cameras, etc. The apparatus may include electrical contacts in the nesting region which mate with contacts integrated into the structure of the chip, to power heating or electrophoresis, for example. The apparatus may be provided with conventional circuitry sensors in communication with sensors in the device for thermal regulation, for example for PCR thermal regulation. The apparatus may also include a computer system comprising a microprocessor for control of the various modules of the system as well as for data analysis.

FIG. 5B shows an exemplary flexible sensor array. Components such as resistors, capacitors and inductors can be printed on the flexible substrate as known by those skilled in the art. Transistors can also be printed. For high speed circuit, a hybrid using active electronics coupled to the flexible electronics can be used. Sensors can be built using these components. The substrate can be planar or non-planar. As used herein, the term “planar substrate” refers to a substrate which extends primarily in two dimensions, while the term “non-planar substrate” refers to a substrate that does not lie essentially in a two dimensional plane and can extend, for example, in a three dimensional orientation. For example, the substrate can include a three dimensional curved or angled (non-planar) housing of a mobile phone, game console, DVD player, computer, wireless modem, and the like. The substrate used with the system can be a planar and/or non-planar preformed molded plastic housing. The substrate also can be made from a variety of materials. Non-limiting examples of substrates include substrates made of acrylonitrile butadiene styrene (ABS), styrene acrylonitrile (SAN), polystyrene, polypropylene, high-density polyethylene (HDPE), low-density polyethylene (LDPE), polyamides, polysulfones, phenolic polymers, acrylics, vinyl polymers, glass, wood, urethanes, epoxies, polyesters, and mixtures thereof. The pores can be configured to form at least one conduit that opens to the outside of the surface of the substrate 13 or to the sensor 16 and extends to a location within the substrate 13 or all the way through the substrate 13. The pores can be any type of pores or pore system, or other similar configuration that allows for a substance to pass therethrough. The pores can be shaped, sized, and/or dimensioned to perform size exclusion selection on the substances that can pass therethrough. That is, the pores can be configured to restrict substances of a certain size from entering into the pores and/or passing from one surface of the substrate 13 to the opposite surface. Accordingly, the pores allow substances smaller than a certain size to enter into the pores. The size of the pores can be configured to be similar to the target substance, which can restrict access to the nanosensors and increase the accuracy of detection when the substrate is used for size exclusion selection. Non-limiting examples of pores sizes include being about, or less than about 0.1 nm, less than about 1 nm, less than about 10 nm, less than about 100 nm, less than about 1 um, less than about 10 um, and less than about 100 um. Additional non-limiting examples of pores sizes include being about 0.01 nm to about 0.1 nm, about 0.1 nm to about 1 nm, about 1 nm to about 10 nm, about 10 nm to about 100 nm, about 100 nm to about 1 um, about 1 um to about 10 um, and about 19 um to about 100 um.

A device can be formed of printed non-volatile memory on polymer. For example, the apparatus can be formed on a printed polymer integrated into packaging material and the integrated processor and memory can perform operations such as monitoring the number and type of touches of the product to determine marketing-relevant information such as attractiveness of the packaged material to consumers. The flexible device 1 can have a non-volatile memory array 11 and a processor 10 integrated with the flexible device 1. The processor 10 is operable to operate in combination with the non-volatile memory array 11 to accumulate information associated with a product. In various applications and contexts, memory systems can include non-volatile memory integrated with a processor or other control logic, and a bus or other communications interface. As non-volatile memories and integrated system continue to evolve, their role in overall systems continue to expand to include various aspects of computation that is facilitated, for example, by phase-change memory in which passage of current switches a memory material between two states, crystalline and amorphous, or additional states that further elevate storage capacity.

In some applications and/or embodiments, the processor 10 can be integrated with non-volatile memory array 11 to form the flexible device 1 which can be further integrated into the product, for example electronic devices, such as mobile and cell phones, notebook computers, personal digital assistants, medical devices, medical diagnostic systems, digital cameras, audio players, digital televisions, automotive and transportation engine control units, USB flash personal discs, and global positioning systems. Accordingly, the flexible device 1 can further include the product integrated with the non-volatile memory array 11 and the processor 10.

In embodiments of the apparatus with processing capability of a processor or other control logic integrated in a distributed manner with non-volatile memory, the processing capability can be implemented with relatively low speed requirement to enable processors to be available in a ubiquitous manner. Accordingly, information can be acquired in a dispersed manner and intercommunicated over vast systems. Thus processors can be inexpensive and memory readily available for various consumer items. Custom versions of memory including non-volatile memory and RAM can be integrated into virtually any product, enabling widespread preprocessing in items such as door handles to determine who has accessed a location and how the access was made to allow any type of processing on the information.

In some embodiments, the flexible device 1 can be configured such that the processor 10 is operable to accumulate and communicate information about use of the product. For example, the apparatus can be used in various types of medical devices to monitor and store aspects of operation. In a particular example embodiment, the apparatus can be used in medical products to form biocompatible electronic products such as electronic devices or medical support materials that can dissolve in a patient's body. Some medical products can be configured to be biocompatible and encapsulated in a textile material, silk, or other suitable substrate that dissolves after a selected time duration. The apparatus can also be constituted in a biodegradable form for implantation including biodegradable circuit components including transistors, diodes, inductors and capacitors that can dissolve in water or in the body.

In another example embodiment, the apparatus can be integrated into a product such as a vehicle, specifically a rental vehicle. For a rental automobile, the apparatus can be configured to monitor use such as distance, speed, or forces acting upon the automobile to ascertain driving behavior of the driver.

A further example application for use of the apparatus can be electrodes for a medical device, such as a Transcutaneous Electrical Nerve Stimulation (TENS) device or any other suitable device. A typical TENS system uses silver electrodes mounted on a fabric or cloth substrate. The apparatus including processor and memory can be integrated into the electrode for monitoring delivery of therapeutic pulses but also to monitor body signals such as electrical signals such as for diagnostic purposes. TENS devices produce electric current to stimulate the nerves for therapeutic purposes at a controlled or modulated pulse width, frequency and intensity. In various embodiments, the apparatus integrated into TENS electrodes includes processing capability that can enable chronic monitoring of biological electrical signals to facilitate diagnostic monitoring as well as therapeutic control.

In further applications and/or embodiments, the flexible device 1 can be constructed with the processor 10 operable to accumulate and communicate information about at least one entity in association with the product. In various embodiments and/or applications, an entity can be a person, a living being, a non-living being, an organization (business, political, or otherwise), a device, a computer, a network, or the like. For purposes of example, the apparatus can be integrated into a biocompatible, biodegradable form for hemodynamic monitoring of pressure and blood flow within the circulatory system. Thus, the processor and integrated memory in the apparatus can enable Holter monitoring of an ambulatory patient independently of any external device, although supporting communication with a device external to the patient's body via telemetry for exchange of commands, instructions, control information, and data.

In still further embodiments, the flexible device 1 can be formed in which the processor 10 is operable to accumulate and communicate information about at least one entity in communication with the product. For example, the apparatus can be integrated into a weather monitoring device such as a thermometer, barometer, anemometer, multi-meter that measures multiple environmental parameters, or the like. The weather monitoring device can include an apparatus that includes a communication interface and sensors integrated with the processor and memory. The weather monitoring device can be in a relatively inaccessible location and can communicate from this location to an entity, such as a weather computer or a person.

In additional example embodiments or applications, the flexible device 1 can be implemented so that the processor 10 is operable to accumulate and communicate information about at least one entity in contact with the product. For example, the apparatus can be integrated with a product in the form of a patient armband in hospitals, identification armband in workplaces or other locations, and the like, for instance to assist in security operations. In another example, the apparatus can be integrated with a product in the form of a soda can or other packaging, for example to assist in automatic or effortless purchase of the product.

In various embodiments, the flexible device 1 can be configured such that the processor 10 is operable to monitor tactile contact with the product. In some applications and/or conditions, tactile contact can be monitored via a tactile sensor accessed by the apparatus that can either be integrated into the apparatus, or the processor can be configured to accept tactile information from a distal sensor. In other applications, tactile information can be sent to the apparatus and processor. In example configurations, the apparatus can be integrated into a product in the form of a steering wheel, joystick, or other control device, and the control logic and memory can be configured to perform precision control operations. In another example embodiment, the apparatus can be integrated into a product in the form of a sports article such as a football, and the control logic and memory can be constructed to detect and identify a person with control of the product, such as identifying who has recovered a fumble.

In a particular example embodiment, the flexible device 1 can be constructed with the processor 10 operable to monitor tactile contact with the product, determine statistics on type, characteristics, and number of occurrences of tactile contact with the product, and store the statistics for access. For example, the apparatus can be integrated into a product in the form of a door handle or door handle sleeve. The processor and memory can be configured to monitor conditions such as who, what, when, and how many people have touched the door handle or sleeve. Some embodiments can monitor how hard the door handle or door handle sleeve is touched.

In various embodiments, the flexible device 1 can include volatile memory (not shown) in combination with the non-volatile memory array 11. Accordingly, in further applications or contexts for embodiments, the flexible device 1 can further include a volatile memory integrated with the non-volatile memory array 11 and the processor 10.

In one embodiment, a processor and flexible memory are integrated on a flexible printed polymer substrate and deployed into a multiple types of products. The device 1 can be composed such that the processor 10 and the non-volatile memory array 11 are integrated onto a printed flexible polymer for integration with the product. In one embodiment, the device 1 integrated onto a printed flexible polymer can be a product in the form of a medical device sleeve or patch, and the control logic and memory configured for use in monitoring implanted medical devices such as knee implants, hip implants, shoulder implants, elbow implants, and the like. The processor and memory can be configured to monitor aspects of performance such as position, angle, angular velocity or acceleration, other dynamics, and the like. In some arrangements, the processor and memory can be configured to assist physical therapy such as measurement of motion. In further arrangements, the processor and memory can be configured to monitor other biological or physiological functions such as blood flow, cardiac performance, hemodynamics, neurological aspects of action, and the like.

Accordingly, a flexible memory can be integrated with processors for further integration into any type of product, even very simple products such as bottles, cans, or packaging materials. A non-volatile memory can be integrated in a system of any suitable product such as, for example, a door handle sleeve to detect and record who, what, when, and how anyone has touched the door handle. Such a system can be used to facilitate access or to provide security. In other examples, a non-volatile memory and processor in some applications with sensors and/or a communication interface can be used in a flexible device for a medical product such as bandages or implants. These products can be formed of dissolvable materials for temporary usage, for example in biocompatible electronic or medical devices that can dissolve in a body environment, or environmental monitors and consumer electronics that can dissolve in compost. Other applications of products incorporating non-volatile memory and processor can include sporting equipment, tags such as for rental cars, patient armbands in hospitals tied to sensors, smart glasses, or any type of device.

In a particular example embodiment and application, the device 1 integrated as a printed flexible polymer can be used for cardiac monitoring such as in the form of a patch that can be attached to a patient's chest or elsewhere on the body. The processor and integrated memory can be used to control continuous monitoring of cardiac signals and activity. The device 1 can enable monitoring, such as by electrocardiography, independently of a separate medical device, although supporting communication and exchange of commands, instructions, and data with an external device.

In further embodiments, instead of a flexible polymer, the non-volatile memory and processor can be formed of silicon that is sufficiently thin to become flexible and thus formed as an inexpensive printed circuit component. Flexible memory in ubiquitous items, using polymer memory or silicon memory, can enable various profitable services, for example in conjunction with medical devices, security services, automotive products, and the like.

In an example embodiment, the apparatus can be integrated into a product in the form of smart glass, magic glass, switchable glass, smart windows or switchable windows for application in windows or skylights, which is electrically switchable glass or glazing which changes light transmission properties when voltage is applied. The apparatus can use the integrated processor and memory to control the amount of light and thus heat transmission. The processor can receive control commands, instructions, and data from a control center or operator, for example to activate the glass to change the glass between transparent and translucent, partially or fully blocking light while maintaining a clear view through the glass, if desired. In some embodiments, the communication interface can be used to report on conditions associated with the window or skylight.

The memory can be selected from a memory integrated circuit or memory chip, register, register file, random access memory (RAM), volatile memory, non-volatile memory, read-only memory, flash memory, ferroelectric RAM (F-RAM), magnetic storage device, disk, optical disk, and the like. In some arrangements, the memory can include multiple types of memory including the non-volatile memory array in the form of multiple types of non-volatile memory technologies, in addition to portions of memory that may be volatile. The memory may include multiple types of memory for use in a redundant fashion. Accordingly, the memory can include two or more memory segments of any non-volatile memory type or technology including read-only memory, flash memory, ferroelectric random access memory (F-RAM), magneto-resistive RAM (M-RAM) or the like. The processor or control logic can operate a segment of M-RAM which is comparable in speed and capacity to volatile RAM while enabling conservation of energy, rapid or instantaneous start-up and shutdown sequences. In other applications, the memory can include memory in the form of charge-coupled devices (CCDs) that are not directly addressable or other pure solid state memory that is reliable and inexpensive for use as separate memory for various applications such as cell phones, and the like.

In some embodiments and/or applications, the apparatus can further include a communication interface integrated with the processor and the non-volatile memory array. The communication interface can be operable for communication with a network. The processor can be operable to perform data preprocessing, history tracking, and manage data and history communication. For example, the apparatus can be integrated into a window and include one or more sensors and communication interface in combination with the processor and memory. The sensor(s) can include a light sensor, a pressure sensor, and a temperature sensor for use in determining conditions that can be monitored and communicated to enable control of a heating and cooling system of a building.

In other embodiments, the apparatus can be integrated to a product in the form of a security device for securing an item such as a home, an automobile, or any other item of value. The apparatus can monitor conditions of the product autonomously of devices external to the product, while supporting updates to the apparatus.

For example, in some embodiments, the apparatus can include both phase change memory (PCRAM) and other memory types and the control logic can assign memory usage according to various operating characteristics such as available power. In a specific example, PCRAM and DRAM may be selected based on power considerations. PCRAM access latencies are typically in the range of tens of nanoseconds, but remain several times slower than DRAM. PCRAM writes use energy-intensive current injection, causing thermal stress within a storage cell that degrades current-injection contacts and limits endurance to hundreds of millions of writes per cell. In an apparatus that uses both PCRAM and DRAM, the control logic can allocate memory usage according to the write density of an application. In an apparatus that includes multiple different types of memory including a spin-transfer M-RAM, the control logic can assign functionality at least in part based on the magnetic properties of memory. In a system that includes at least one portion of F-RAM, the control logic can exploit operating characteristics of extremely high endurance, very low power consumption (since F-RAM does not require a charge pump like other non-volatile memories), single-cycle write speeds, and gamma radiation tolerance. The apparatus can include different segments of different types of memory including volatile and non-volatile memory, flash, dynamic RAM (DRAM) and the like, and use the control logic to attain different performance/cost benefits. In embodiments adapted to promote write durability, the apparatus can include a non-volatile memory array with multiple types of memory including at least one portion of memory characterized by elevated write endurance. In a particular embodiment, the non-volatile memory array can include at least on portion formed of M-RAM which is based on a tunneling magneto-resistive (TMR) effect. The individual M-RAM memory cells include a magnetic tunnel junction (MTJ) which can be a metal-insulator-metal structure with ferromagnetic electrodes. A small bias voltage applied between the electrode causes a tunnel current to flow. The MTJ is exposed to an external magnetic field and forms a hysteresis loop with two stable states, corresponding to 0 and 1 data states at zero magnetic field. M-RAM is characterized among non-volatile memory technologies as having excellent write endurance with essentially no significant degradation in magneto-resistance or tunnel junction resistance through millions of write cycles. Accordingly, the control logic can monitor and determine whether a particular application or process is characterized by frequent, enduring write operations and assign a portion of M-RAM to handle memory accesses. Another memory technology characterized by write endurance is ferroelectric RAM (FeRAM). FeRAM can be constructed using material such as lead-zirconate-titanate (PZT), strontium-bismuth-tantalate (SBT), lanthanum substituted bismuth-tantalate (BLT), and others. An externally applied electric field causes polarization of the FeRAM material to be switched and information retained even upon removal of the field. In absence of the electric field, polarization has two distinct stable states to enable usage in memory storage. FeRAM can have write endurance at the level of M-RAM and is further characterized by a reduced cell size and thus higher density. Thus, the control logic can monitor and determine whether a particular application or process is characterized by frequent, enduring write operations in combination with a relatively large number of storage cells. The control logic can assign a portion of FeRAM to handle memory accesses. The control logic can be a processor, a distributed-circuitry processor, a processing unit, a processing unit distributed over memory, arithmetic logic and associated registers, a microprocessor, a graphics processing unit, a physics processing unit, a signal processor, a network processor, a front-end processor, a state machine, a coprocessor, a floating point unit, a data processor, a word processor, and the like.

The apparatus can include any suitable type of sensor such as motion or position sensors, electrical signal sensors, pressure sensors, oxygen sensors, and the like. The processor and memory can be configured to facilitate monitoring for therapeutic and diagnostic purposes, and delivery of therapy. The control logic can be operable to perform maintenance operations including information handling in the memory in response to physical phenomena imposes on the memory. For example, the memory device can incorporate sensors or other components that detect phenomena which can be monitored by the control logic to detect magnetic fields, temperature, velocity, rotation, acceleration, inclination, gravity, humidity, moisture, vibration, pressure, sound, electrical fields or conditions such as voltage, current, power, resistance, and other physical aspects of the environment to enable the control logic to perform actions to maintain, repair, clean, or other operations applied to the memory.

In some embodiments and/or applications, the apparatus can receive information via the optical link, independently of the system bus connected to a processor, and the apparatus can use the extra-bus information to perform management or housekeeping functions to track applications and/or processes (or, for example, bit correction) via data sent optically to the apparatus. The optical link thus enables low-bandwidth, back-channel communication, enabling formation of a memory that can communicate with large bursts of data for placement with optical accessibility. For example, an optical sensor or silicon-based optical data connection can use silicon photonics and a hybrid silicon laser for communication between integrated circuit chips at distributed locations using plasmons (quanta of plasma oscillation) to communicate over relatively long distances, for example 2-3 inches on a narrow nano-wire coupler. The plasmon is a quasi-particle that results from quantization of plasma oscillations. Data can be received and converted using an optical antenna, a nano-cavity, or a quantum dot. The communication field can travel independently of a wired bus structure.

In some embodiments, the apparatus can be configured to respond to time signals. In various embodiments and/or arrangements, the time signal can be selected from among a visible, audible, mechanical, or electronic signal used as a reference to determine time, a clock, a timing pulse, and the like. Workload can refer to impact on the memory device, portions of memory within the memory device, the system containing the memory device, or any predetermined scope relative to the memory device, or the like. Workload can be analyzed and managed according to any selected workload parameters such as memory capacity, memory portion, memory type, memory characteristics, memory operating characteristics, memory availability, processor speed, logic speed, interface or network latency, potential workloads in queue, remaining battery life, energy cost, temperature, location, server type, affinity information, processing time, and the like.

Some embodiments can implement a pseudo-random number generator coupled to the hybrid memory and coupled to the logic operable to perform encryption operations. The pseudo-random number generator can be operable to generate numbers for usage in encrypting information. The medical information handling system can be configured to implement one or more of a variety of security schemes including channel encryption, storage encryption, RSA (Rivest, Shamir, Adleman) cryptography and key distribution, Public Key Infrastructure (PKI). Accordingly, the logic operable to perform encryption operations can be operable to perform stream encryption of communicated information wherein processor and memory sides are assigned a key. In another example functionality, the logic operable to perform encryption operations can be operable to encrypt information that is storage encrypted wherein the storage-encrypted information is encrypted by the processor, stored in the hybrid memory, accessed from the hybrid memory, and decrypted by the processor.

In some embodiments and/or applications, the information handling system can be configured to use of cryptographic processing to facilitate information handling. For example, data can be copied for redundant storage and the redundant copy can be secured by encryption and stored in the non-volatile memory in encrypted form. The encrypted redundant copy of the data can be used for restoration in the event of a detected error. In another example, A cryptographic hash function generates information indicative of data integrity, whether changes in data are accidental or maliciously and intentional. Modification to the data can be detected through a mismatching hash value. For a particular hash value, finding of input data that yields the same hash value is not easily possible, if an attacker can change not only the message but also the hash value, then a keyed hash or message authentication code (MAC) can supply additional security. Without knowing the key, for the attacker to calculate the correct keyed hash value for a modified message is not feasible.

In one embodiment, a humidity sensor employs a capacitor with a metal material such as copper or silver with a printed humidity sensitive polymer poly (2-hydroxyethyl methacrylate) (pHEMA). In this embodiment, the layer of pHEMA can be at the bottom, followed by the metal material, and by another layer of pHEMA. In another embodiment, the capacitor can be a silver or copper base with interdigitated arms formed above the base, and in this embodiment, the pHEMA is applied on one layer above the metal material. The sensor provides a capacitive response to the humidity. Various types of humidity-sensitive polymers containing doped cations or anions, quarternary ammonium, phosphonium salt and sulfonic acid—containing polyelectrolytes can be used for humidity sensing. Various conducting polymers such as polyaniline, polypyrrole and polythiophene can be used. Other materials include NaPSS: Sodium polystyrenesulfonate; DEAMA-co-BMA: Poly(N,N-diethylaminoethyl methacrylate-cobutyl methacrylate); MAPTAC: [3-(methacrylamino)propyl]trimethyl ammonium chloride; MSMA: 3-(trimethoxysilyl)propyl methacrylate; MMA: Methyl methacrylate; AEPAB: [2-(acryloyloxy)ethyl]dimethylpropyl ammonium bromide; PS: Polystyrene; HEMA: 2-hydroxyethylmethacrylate; BPA: 4-acryloyloxybenzophenone; PANI: Polyaniline; PVA: Polyvinyl alcohol: PSSA: Poly(styrenesulfonic acid); PVAc-co-BuAcry: Poly(vinyl acetate-cobutylacrylate); VTBPC: Vinylbenzyltributylphosphonium chloride; METAC: [2-(methacryloyloxy)ethyl]trimethyl ammonium chloride; 2-EHA: 2-ethylhexylacrylate; 4-VP: 4-vinyl pyridine; MEDPAB: [2-(methacryloyloxy)ethyl]dimethylpropyl ammonium bromide; TSPM: 3-(trimethoxysilyl)propyl methacrylate; AMPS: Poly(2-acrylamido-2-methylpropane sulfonate); HMPTAC: 2-Hydroxy-3-methacryloxypropyltrimethylammonium chloride; PEG: Polyethylene glycol.

A printed temperature sensor can be a printed resistor with a positive temperature coefficient (PTC) or a negative temperature coefficient (NTC). To reduce impact of strain on the temperature sensor, in one embodiment, a temperature dependent resistor is formed in series with a temperature independent resistor, which is of similar construction and hence has a similar response to strain caused by mechanical force applied to a region of a sensing device including both resistors. By measuring variations in the potential difference across the temperature independent resistor, the mechanical distortion of the sensor can be determined. This information can be used to correct a measurement of the potential difference across the temperature dependent resistor, which indicates the change in temperature. Thus, in the case of a temperature sensor, the temperature reading of the sensor is automatically corrected for mechanical distortion (strain) of the sensor.

A touch sensor can be formed with a printed dielectric material layered between electrodes. While the touch sensor is illustrated as a single dielectric layered between two electrodes, it is to be understood that the touch sensor can include additional dielectric and electrode layers, depending on the design of the touch sensor. In an example, electrode can be the same material as electrode. In another example, electrode can be a different material from electrode. The dielectric and the electrodes can be formed of a polymer, such as a flexible polymer. The polymer may also be an amorphous polymer. In examples, the polymer can be a silicone, such as polydimethylsiloxane (PDMS). Furthermore, the electrodes can be a silicone and a conducting medium, such as carbon, or any other suitable conducting material, compounded into the silicone. When forming the touch sensor to a curved surface, regions of the touch sensor may deform more than other regions of the touch sensor, changing the capacitance of these deformed regions as compared to the less deformed regions of the touch sensor. By calibrating the touch sensor after forming the touch sensor to the curved surface, this change in capacitance can be negated. The touch sensor additionally supports a strain up to 400%, such as up to 350%. This high supported strain enables the force/deflection curve of the touch sensor to be made less sensitive when compared to a more rigid touchpad. In this sense, sensitivity relates to the force versus the deflection of the touch sensor. When a sensor is very stiff, a large force causes a small deflection in the sensor, making the sensor 200 very responsive to small deflections. This responsiveness to small deflection makes the input hard to control for the user. However, when the force is low and a large strain results due to the low modulus sensor material, the change of capacitance is large, resulting in a large signal input, so the user has greater control of the input signal by applying a force to the touch sensor (i.e., the sensor is less sensitive) and the touch sensor is less prone to errors. The capacitance of the touch sensor is changed by deforming the touch sensor. In some cases, deforming the touch sensor means applying pressure to the touch sensor such that the shape of the touch sensor is altered. Capacitance is a function of the electrode area A, the electrode charge, the distance d between electrodes, and the permittivity of the volume between charge plates. When a force is exerted on the touch sensor, the electrode area A deforms and the distance d changes, which in turn changes the capacitance of the touch sensor. The capacitance is sensed by a circuit (not illustrated) and correlated to a force applied to the touch sensor.

The device 1 can be powered by a flexible battery such as lithium-ion battery with a negative electrode, or anode, and a positive electrode, or cathode, coated on a metal foil current collector. Between these electrodes is a thin polymer separator, which keeps the electrodes from touching and allows lithium ions to pass though during charging or discharging. The metal foil current collectors are formed as Chemical Vapor Deposition (CVD)-grown carbon nanotube mats. Carbon nanotubes are highly conductive and extremely strong—two features a flexible battery would need in order to generate power in diverse forms. A separator is placed between a carbon nanotube-based anode and cathode that they then encapsulated in a thin, flexible plastic film.

FIGS. 5C-5D show exemplary clothing with flexible circuits thereon. Accelerometers, temperature sensors, EKG sensors, EMG sensors, and other sensors can be formed on the flexible clothing. One major symptom of a stroke is unexplained weakness or numbness in the muscle. To detect muscle weakness or numbness, in one embodiment, the system applies a pattern recognizer such as a neural network or a Hidden Markov Model (HMM) to analyze accelerometer output. In another embodiment, electromyography (EMG) is used to detect muscle weakness. In another embodiment, EMG and a pattern analyzer is used to detect muscle weakness. In yet another embodiment, a pattern analyzer analyzes both accelerometer and EMG data to determine muscle weakness. In a further embodiment, historical ambulatory information (time and place) is used to further detect changes in muscle strength. In yet other embodiments, accelerometer data is used to confirm that the patient is at rest so that EMG data can be accurately captured or to compensate for motion artifacts in the EMG data in accordance with a linear or non-linear compensation table. In yet another embodiment, the EMG data is used to detect muscle fatigue and to generate a warning to the patient to get to a resting place or a notification to a nurse or caregiver to render timely assistance. The amplitude of the EMG signal is stochastic (random) in nature and can be reasonably represented by a Gausian distribution function. The amplitude of the signal can range from 0 to 10 mV (peak-to-peak) or 0 to 1.5 mV (rms). The usable energy of the signal is limited to the 0 to 500 Hz frequency range, with the dominant energy being in the 50-150 Hz range. Usable signals are those with energy above the electrical noise level. The dominant concern for the ambient noise arises from the 60 Hz (or 50 Hz) radiation from power sources. The ambient noise signal may have an amplitude that is one to three orders of magnitude greater than the EMG signal. There are two main sources of motion artifact: one from the interface between the detection surface of the electrode and the skin, the other from movement of the cable connecting the electrode to the amplifier. The electrical signals of both noise sources have most of their energy in the frequency range from 0 to 20 Hz and can be reduced. To eliminate the potentially much greater noise signal from power line sources, a differential instrumentation amplifier can be attached to the flexible substrate. Any signal that originates far away from the detection sites will appear as a common signal, whereas signals in the immediate vicinity of the detection surfaces will be different and consequently will be amplified. Thus, relatively distant power lines noise signals will be removed and relatively local EMG signals will be amplified. The source impedance at the junction of the skin and detection surface may range from several thousand ohms to several megohms for dry skin. In order to prevent attenuation and distortion of the detected signal due to the effects of input loading, the input impedance of the differential amplifier is as large as possible, without causing ancillary complications to the workings of the differential amplifier. The signal to noise ratio is increased by filtering between 20-500 Hz with a roll-off of 12 dB/octave.

In one embodiment, direct EMG pre-amplification at the skin surface provides the best myoelectric signal quality for accurate, reliable EMG signal detection and eliminates cable motion artifact. The double-differential instrumentation pre-amplifier design attenuates unwanted common-mode bioelectric signals to reduce cross-talk from adjacent muscle groups. Internal RFI and ESD protection prevents radio frequency interference and static damage. The constant low-impedance output of the pre-amplifier completely eliminates cable noise and cable motion artifacts without requiring any additional signal processing within the pre-amplifier. An integral ground reference plane provides immunity to electromagnetic environmental noise. All signal and power conductors in the pre-amplifier cable are enclosed inside an independent, isolated shield to eliminate interference from AC power-lines and other sources. The contacts are corrosion-free, medical grade stainless steel for maximal signal flow. The system uses biocompatible housing and sensor materials to prevent allergic reactions.

In another implementation, a micro-powered EMG embodiment includes an instrumentation amplifier and an AC coupling that maintains a high CMRR with a gain of about 1000. The electronic circuits are mounted on a flexible circuit board (FPC) with slidable electrode settings that allows differential recording at various distances between the electrodes. The high gain amplifier is placed next to the recording electrodes to achieve high SNR. Battery power provides isolation and low noise at various frequencies that would likely not be fully attenuated by the PSRR and causing alias errors.

The system can detect dominant symptoms of stroke can include weakness or paralysis of the arms and/or legs, incoordination (ataxia), numbness in the arms/legs using accelerometers or EMG sensors. The EMG sensors can detect muscle fatigue and can warn the patient to get to a resting area if necessary to prevent a fall. The system can detect partial/total loss of vision by asking the patient to read a predetermined phrase and detect slur using speech recognizer. The system can detect loss of consciousness/coma by detecting lack of movement. Voice/speech disturbances are not initially the dominant symptoms in stroke, and the disturbances can be detected by a speech recognizer. In one implementation, the system uses PNL (probabilistic networks library) to detect unusual patient movement/ambulatory activities that will lead to a more extensive check for stroke occurrence. PNL supports dynamic Bayes nets, and factor graphs; influence diagrams. For inference, PNL supports exact inference using the junction tree algorithm, and approximate inference using loopy belief propagation or Gibbs sampling. Learning can be divided along many axes: parameter or structure, directed or undirected, fully observed or partially observed, batch or online, discriminative or maximum likelihood, among others. First, the system performs data normalization and filtering for the accelerometers and EMG sensors that detect patient movements and muscle strength. The data can include in-door positioning information, 3D acceleration information, or EMG/EKG/EEG data, for example. The data can be processed using wavelet as discussed above or using any suitable normalization/filtering techniques. Next, the system performs parameterization and discretization. The Bayesian network is adapted in accordance with a predefined network topology. The system also defines conditional probability distributions. The system then generates the probability of event P(y), under various scenarios. Training data is acquired and a training method is built for the Bayesian network engine. Next, the system tunes model parameters and performs testing on the thus formed Bayesian network.

In one embodiment, a housing (such as a strap, a wrist-band, or a patch) provides a plurality of sensor contacts for EKG and/or EMG. The same contacts can be used for detecting EKG or EMG and can be placed as two parallel contacts (linear or spot shape) on opposite sides of the band, two adjacent parallel contacts on the inner surface of the band, two parallel adjacent contacts on the back of the wrist-watch, or alternatively one contact on the back of the watch and one contact on the wrist-band. The outputs of the differential contacts are filtered to remove motion artifacts. The differential signal is captured, and suitably filtered using high pass/low pass filters to remove noise, and digitized for signal processing. In one embodiment, separate amplifiers are used to detect EKG (between 50 mHz and 200 Hz) and for EMG (between 10 Hz and 500 Hz). In another embodiment, one common amp is used for both EKG/EMG, and software filter is applied to the digitized signal to extract EKG and EMG signals, respectively. The unit can apply Wavelet processing to convert the signal into the frequency domain and apply recognizers such as Bayesian, NN or HMM to pull the EMG or EKG signals from noise. The system uses a plurality of wireless nodes to transmit position and to triangulate with the mobile node to determine position. 3D accelerometer outputs can be integrated to provide movement vectors and positioning information. Both radio triangulation and accelerometer data can confirm the position of the patient. The RF signature of a plurality of nodes with known position can be used to detect proximity to a particular node with a known position and the patient's position can be extrapolated therefrom.

In one embodiment, Analog Device's AD627, a micro-power instrumentation amplifier, is used for differential recordings while consuming low power. In dual supply mode, the power rails Vs can be as low as ±1.1 Volt, which is ideal for battery-powered applications. With a maximum quiescent current of 85 μA (60 μA typical), the unit can operate continuously for several hundred hours before requiring battery replacement. The batteries are lithium cells providing 3.0V to be capable of recording signals up to +1 mV to provide sufficient margin to deal with various artifacts such as offsets and temperature drifts. The amplifier's reference is connected to the analog ground to avoid additional power consumption and provide a low impedance connection to maintain the high CMRR. To generate virtual ground while providing low impedance at the amplifier's reference, an additional amplifier can be used. In one implementation, the high-pass filtering does not require additional components since it is achieved by the limits of the gain versus frequency characteristics of the instrumentation amplifier. The amplifier has been selected such that with a gain of 60 dB, a flat response could be observed up to a maximum of 100 Hz with gain attenuation above 100 Hz in one implementation. In another implementation, a high pass filter is used so that the cut-off frequency becomes dependent upon the gain value of the unit. The bootstrap AC-coupling maintains a much higher CMRR so critical in differential measurements. Assuming that the skin-electrode impedance may vary between 5 K- and 10 K-ohms, 1 M-ohm input impedance is used to maintain loading errors below acceptable thresholds between 0.5% and 1%.

When an electrode is placed on the skin, the detection surfaces come in contact with the electrolytes in the skin. A chemical reaction takes place which requires some time to stabilize, typically in the order of a few seconds. The chemical reaction should remain stable during the recording session and should not change significantly if the electrical characteristics of the skin change from sweating or humidity changes. The active electrodes do not require any abrasive skin preparation and removal of hair. The electrode geometry can be circular or can be elongated such as bars. The bar configuration intersects more fibers. The inter detection-surface distance affects the bandwidth and amplitude of the EMG signal; a smaller distance shifts the bandwidth to higher frequencies and lowers the amplitude of the signal. An inter detection-surface of 1.0 cm provides one configuration that detects representative electrical activity of the muscle during a contraction. The electrode can be placed between a motor point and the tendon insertion or between two motor points, and along the longitudinal midline of the muscle. The longitudinal axis of the electrode (which passes through both detection surfaces) should be aligned parallel to the length of the muscle fibers. The electrode location is positioned between the motor point (or innervation zone) and the tendinous insertion, with the detection surfaces arranged so that they intersect as many muscle fibers as possible.

In one embodiment, a multi-functional bio-data acquisition provides programmable multiplexing of the same differential amplifiers for extracting EEG (electroencephalogram), ECG (electrocardiogram), or EMG (electromyogram) waves. The system includes an AC-coupled chopped instrumentation amplifier, a spike filtering stage, a constant gain stage, and a continuous-time variable gain stage, whose gain is defined by the ratio of the capacitors. The system consumes microamps from 3V. The gain of the channel can be digitally set to 400, 800, 1600 or 2600. Additionally, the bandwidth of the circuit can be adjusted via the bandwidth select switches for different biopotentials. The high cut-off frequency of the circuit can be digitally selected for different applications of EEG acquisition.

In another embodiment, a high-resolution, rectangular, surface array electrode-amplifier and associated signal conditioning circuitry captures electromyogram (EMG) signals. The embodiment has a rectangular array electrode-amplifier followed by a signal conditioning circuit. The signal conditioning circuit is generic, i.e., capable of receiving inputs from a number of different/interchangeable EMG/EKG/EEG electrode-amplifier sources (including from both monopolar and bipolar electrode configurations). The electrode-amplifier is cascaded with a separate signal conditioner minimizes noise and motion artifact by buffering the EMG signal near the source (the amplifier presents a very high impedance input to the EMG source, and a very low output impedance); minimizes noise by amplifying the EMG signal early in the processing chain (assuming the electrode-amplifier includes signal gain) and minimizes the physical size of this embodiment by only including a first amplification stage near the body. The signals are digitized and transmitted over a wireless network such as WiFI, Zigbee, or Bluetooth transceivers and processed by the base station that is remote from the patient. For either high-resolution monopolar arrays or classical bipolar surface electrode-amplifiers, the output of the electrode-amplifier is a single-ended signal (referenced to the isolated reference). The electrode-amplifier transduces and buffers the EMG signal, providing high gain without causing saturation due to either offset potentials or motion artifact. The signal conditioning circuit provides selectable gain (to magnify the signal up to the range of the data recording/monitoring instrumentation, high-pass filtering (to attenuate motion artifact and any offset potentials), electrical isolation (to prevent injurious current from entering the subject) and low-pass filtering (for anti-aliasing and to attenuate noise out of the physiologic frequency range).

The EMG signal can be rectified, integrated a specified interval of and subsequently forming a time series of the integrated values. The system can calculate the root-mean-squared (rms) and the average rectified (avr) value of the EMG signal. The system can also determine muscle fatigue through the analysis of the frequency spectrum of the signal. The system can also assess neurological diseases which affect the fiber typing or the fiber cross-sectional area of the muscle. Various mathematical techniques and Artificial Intelligence (Al) analyzer can be applied. Mathematical models include wavelet transform, time-frequency approaches, Fourier transform, Wigner-Ville Distribution (WVD), statistical measures, and higher-order statistics. Al approaches towards signal recognition include Artificial Neural Networks (ANN), dynamic recurrent neural networks (DRNN), fuzzy logic system, Genetic Algorithm (GA), and Hidden Markov Model (HMM).

A single-threshold method or alternatively a double threshold method can be used which compares the EMG signal with one or more fixed thresholds. The embodiment is based on the comparison of the rectified raw signals and one or more amplitude thresholds whose value depends on the mean power of the background noise. Alternatively, the system can perform spectrum matching instead of waveform matching techniques when the interference is induced by low frequency baseline drift or by high frequency noise.

EMG signals are the superposition of activities of multiple motor units. The EMG signal can be decomposed to reveal the mechanisms pertaining to muscle and nerve control. Decomposition of EMG signal can be done by wavelet spectrum matching and principle component analysis of wavelet coefficients where the signal is de-noised and then EMG spikes are detected, classified and separated. In another embodiment, principle components analysis (PAC) for wavelet coefficients is used with the following stages: segmentation, wavelet transform, PCA, and clustering. EMG signal decomposition can also be done using non-linear least mean square (LMS) optimization of higher-order cumulants.

Time and frequency domain approaches can be used. The wavelet transform (WT) is an efficient mathematical tool for local analysis of non-stationary and fast transient signals. One of the main properties of WT is that it can be implemented by means of a discrete time filter bank. The Fourier transforms of the wavelets are referred as WT filters. The WT represents a very suitable method for the classification of EMG signals. The system can also apply Cohen class transformation, Wigner-Ville distribution (WVD), and Choi-Williams distribution or other time-frequency approaches for EMG signal processing.

In Cohen class transformation, the class time-frequency representation is particularly suitable to analyze surface myoelectric signals recorded during dynamic contractions, which can be modeled as realizations of nonstationary stochastic process. The WVD is a time-frequency that can display the frequency as a function of time, thus utilizing all available information contained in the EMG signal. Although the EMG signal can often be considered as quasi-stationary there is still important information that is transited and may be distinguished by WVD. Implementing the WVD with digital computer requires a discrete form. This allows the use of fast Fourier transform (FFT), which produces a discrete-time, discrete-frequency representation. The common type of time frequency distribution is the Short-time Fourier Transform (STFT). The Choi-Williams method is a reduced interference distribution. The STFT can be used to show the compression of the spectrum as the muscle fatigue. The WVD has cross-terms and therefore is not a precise representation of the changing of the frequency components with fatigue. When walls appear in the Choi-William distribution, there is a spike in the original signal. It will decide if the walls contain any significant information for the study of muscle fatigue. In another embodiment, the autoregressive (AR) time series model can be used to study EMG signal. In one embodiment, neural networks can process EMG signal where EMG features are first extracted through Fourier analysis and clustered using fuzzy c-means algorithm. Fuzzy c-means (FCM) is a method of clustering which allows data to belong to two or more clusters. The neural network output represents a degree of desired muscle stimulation over a synergic, but enervated muscle. Error-back propagation method is used as a learning procedure for multilayred, feedforward neural network. In one implementation, the network topology can be the feedforward variety with one input layer containing 256 input neurodes, one hidden layer with two neurodes and one output neurode. Fuzzy logic systems are advantageous in biomedical signal processing and classification. Biomedical signals such as EMG signals are not always strictly repeatable and may sometimes even be contradictory. The experience of medical experts can be incorporated. It is possible to integrate this incomplete but valuable knowledge into the fuzzy logic system, due to the system's reasoning style, which is similar to that of a human being. The kernel of a fuzzy system is the fuzzy inference engine. The knowledge of an expert or well-classified examples are expressed as or transferred to a set of “fuzzy production rules” in the form of IF-THEN, leading to algorithms describing what action or selection should be taken based on the currently observed information. In one embodiment, higher-order statistics (HOS) is used for analyzing and interpreting the characteristics and nature of a random process. The subject of HOS is based on the theory of expectation (probability theory).

In addition to stroke detection, EMG can be used to sense isometric muscular activity (type of muscular activity that does not translate into movement). This feature makes it possible to define a class of subtle motionless gestures to control interface without being noticed and without disrupting the surrounding environment. Using EMG, the user can react to the cues in a subtle way, without disrupting their environment and without using their hands on the interface. The EMG controller does not occupy the user's hands, and does not require them to operate it; hence it is “hands free”. The system can be used in interactive computer gaming which would have access to heart rate, galvanic skin response, and eye movement signals, so the game could respond to a player's emotional state or guess his or her level of situation awareness by monitoring eye movements. EMG/EEG signal can be used for man-machine interfaces by directly connecting a person to a computer via the human electrical nervous system. Based on EMG and EEG signals, the system applies pattern recognition system to interpret these signals as computer control commands. The system can also be used for Mime Speech Recognition which recognizes speech by observing the muscle associated with speech and is not based on voice signals but EMG. The MSR realizes unvoiced communication and because voice signals are not used, MSR can be applied in noisy environments; it can support people without vocal cords and aphasics. In another embodiment, EMG and/or electroencephalogram (EEG) features are used for predicting behavioral alertness levels. EMG and EEG features were derived from temporal, frequency spectral, and statistical analyses. Behavioral alertness levels were quantified by correct rates of performance on an auditory and a visual vigilance task, separately. A subset of three EEG features, the relative spectral amplitudes in the alpha (alpha %, 8-13 Hz) and theta (theta %, 4-8 Hz) bands, and the mean frequency of the EEG spectrum (MF) can be used for predicting the auditory alertness level.

In yet a further embodiment for performing motor motion analysis, an HMM is used to determine the physical activities of a patient, to monitor overall activity levels and assess compliance with a prescribed exercise regimen and/or efficacy of a treatment program. The HMM may also measure the quality of movement of the monitored activities. For example, the system may be calibrated or trained in the manner previously described, to recognize movements of a prescribed exercise program. Motor function information associated with the recognized movements may be sent to the server for subsequent review. A physician, clinician, or physical therapist with access to patient data may remotely monitor compliance with the prescribed program or a standardized test on motor skill. For example, patients can take the Wolf Motor Function test and acceleration data is captured on the following tasks:

placing the forearm on a table from the side

moving the forearm from the table to a box on the table from the side

extending the elbow to the side

extending the elbow to the side against a light weight

placing the hand on a table from the front

moving the hand from table to box

flexing the elbow to retrieve a light weight

lifting a can of water

lifting a pencil, lifting a paper clip

stacking checkers, flipping cards

turning a key in a lock

folding a towel

lifting a basket from the table to a shelf above the table.

FIG. 5E shows an exemplary diaper with flexible circuits thereon. In one embodiment, the diaper receives a deposit of capacitive or resistive sensors that detect soiling and communicating the amount of soiling via RF means to a monitoring station for diaper change.

Urine Handling with microneedles as one way valves is detailed next. In addition to a conventional diaper with superabsorbent crystals, microneedle valves are used to minimize backflow and odor. Urine flows down microneedles as a urine catcher. The urine then passes through a sealing liquid, such as a designed oil based fluid or vegetable oil, and collects in the reservoir below. The different densities of urine and oil (urine is denser than oil—oil floats!) mean that the urine sinks through the sealing liquid and the oil floats on top of the layer of urine below. Any air bubbles rise to the top and escape leaving the urine in a relatively low oxygen environment. Odor is therefore trapped below the oil layer and odor is eliminated. Preferably, the system is designed to slow the urine before it hits the oil so that laminar flow displacement doesn't move the oil to the bottom. After catching the urine in the reservoir, an outlet is provided to dispose urine into the toilet plumbing system. In one embodiment, to increase urine capacity, multiple urine tanks can be formed around the body of the underwear and a pump can be used to move the urine to different tanks for balance. Each tank includes a drain outlet that is joined at a master outlet so that a single valve can be used to dispose urine into the toilet plumbing system. There are two embodiments: cartridge based and non-cartridge based units. Cartridge based units use a replaceable cartridge pre-filled with sealing liquid. These units are periodically replaced as the sealing liquid is slowly eroded or degraded. Non Cartridge based systems work by simply introducing the sealing liquid into the drain hole and allowing it to naturally settle into the correct position.

In yet another implementation, the odor trapping is controlled electronically using a liquid detector and valve or clamp that is opened when urination is detected but otherwise is closed. In one implementation, a pump can be used to move urine into a storage chamber embedded in the front or back regions to provide high storage capacity. The urine chamber has an electronically controlled discharge valve so the user can wirelessly dump the urine without touching the urine container when the user subsequently visits a toilet. The user can also manually discharge the urine if the wireless control is not available or if needed for any reasons.

To handle fecal matters, a disposable biodegradable pad is placed under the anus, and an expandable container or bellow is used to capture fecal matters. When not needed, the bellow is compressed into a small volume. During use, the bellow expands to capture the fecal matters. When the session is done, the wearer moves to a toilet, uses the hand in a wiping motion to clean him/herself and at the end of the motion the liner/bag is released into the toilet. Thus, in one action, the pad with the accordion bag is disposed while the body is cleansed. When the fecal is a solid, cleaning is easy. However, when the fecal matter is liquid or chunky, cleaning is quite challenging. To actively capture liquid fecal matters, a pump is used to suck the liquid into the bellow/container. Upon detection of liquid exiting the anus, the pump is activated and causes the pad to form a seal around butt and to suck the liquid fecal into the accordion disposable bellow. In another embodiment that is quieter than the pump embodiment, to provide an electrostatic force that delivers liquid fecal matter into the bellow container, the pad can be negatively charged, while the bellow can be positively charged. In another embodiment, both the pump and the electrostatic differential can be used to forcefully urge liquid fecal matter to into the accordion like bellow container. An active directed movement of liquid fecal matter when the wearer is about to have a bowel movement minimizes skin rash and other medical problem if the skin is exposed to waste materials for an extended duration.

In one embodiment, an odor control dispenser such as fragrance fluid dispenser or solid dispenser can be activated to neutralize odor at the point of use. A highly concentrated plant extract can be used to avoid polluting the environment with eucalyptus, floral oasis and refreshing spring flavors.

In another embodiment, the fecal storage pad and bellow container, including the other parts identified above, is biodegradable or, preferably, formed from a substance that may dissolve or disintegrate in water so that the fecal and the entire chamber may be flushed in the toilet after use. Samples of such material include, paper and other cellulosic materials, materials formed substantially from starch, gum, or alginate material such as agar and so on.

In one embodiment, a user facing layer may be formed of Duratex™, which is an aperture film with a non-woven scrim (AFW/NW) layer attached. Aperture film has small holes in it the shape of a funnel, which helps to move fluid in only one direction. Non-woven fibers passing over the holes of the aperture film, and the film is oriented such that the apertures point away from the body, allowing fluid to pass into the lower layers, but not to return. A second layer uses a through air bonded (TAB) material similar to bleeder/breather that is used in the composite industry and allows nesting of the apertures and the spreading of fluids to the manifold. A third layer, an aperture film, is the start of the manifold. A porcupine type roller may be used to form the aperture film for forming the number of holes, or such holes may be punched or otherwise machine formed. The number of holes may be varied to determine optimum performance of the apparatus. A fourth layer forms the center of the manifold and may comprise either TAB or bleeder/breather, a polyester non-woven fabric. The density of material may be increased around the tube exit area. In any event, the manifold nests in this material. The last layer is an outside layer back sheet that can be a treated breathable sheet or breathable polyethylene (PE) film. The edges of the article may be sealed together by heat bonding, melting adhesive (e.g., hot glue), air stitch, or other methods.

A processor or CPU detects urine liquid by determining two electrodes are shorted when the urine flows through the electrodes. The CPU activates a clamp to allow the urine to flow into a reservoir 103. When urine is not present, the claim returns to its normally closed position to cut off odor and urine from getting out of the reservoir. Subsequently, when the user is at a toilet, the user can wirelessly instruct the CPU 101 to open the urine out valve to dump the urine into the toilet. The command can be from a smart phone, smart watch, or smart wearable device that transmits the command over WiFi, Bluetooth, Zigbee, or other wired media, for example. Once the command is received, the urine reservoir content is dumped out and the reservoir can be reused again to relieve the user when needed, yet avoiding dumping diaper into the landfill each time s/he urinates with a diaper.

A transceiver provides a portable wireless incontinence monitoring system for aged care facilities. Benefits of remote monitoring include increasing quality of life for the elderly and reducing the work load of caregivers. The system detects and accurately measures the voided volume for each event. A strip with an array of sensors is placed in a diaper to measure conductivity of urine. Sensors capture volume sizes, timing between each event and the number of urinary events per day. In one embodiment, an incontinence monitoring system includes a sensor placed into the article, and connected to the system of FIG. 1C which is placed in the patients' underwear. The wireless transceiver 107 transmits the sensors' data to a server which collects all the data from all in an aged care facility. The recorded data is then analyzed by software and the results are shown to the end user via a user interface. The caregivers can check the residents' status from any workstation in communication with the server to see if the resident has to be changed or not. Also, an alert can be sent to a caregiver's mobile telephone, tablet computer or other mobile communication device. The system provides a process for caregivers to take care of residents while maintaining user comfort. Caregivers need to only create a profile for each resident with the user interface via any workstation. This enables the system to keep track of each resident and alert the caregivers when a resident's underwear or diaper has to be changed.

In another embodiment, a camera can be used to capture patient data. For a stool analysis, a stool sample is collected in the container and analyzed by camera and sensor(s). The camera analysis includes microscopic examination, chemical tests, and microbiologic tests. The stool is checked for color, consistency, weight (volume), shape, odor, and the presence of mucus. The stool may be examined for hidden (occult) blood, fat, meat fibers, bile, white blood cells, and sugars called reducing substances.

Human fecal matter varies significantly in appearance, depending on diet and health. In one embodiment, the camera classifies stools using the Bristol stool scale which is a medical aid designed to classify the form of human feces into seven categories. Developed by K. W. Heaton at the University of Bristol, the seven types of stool are: Separate hard lumps, like nuts (hard to pass), Sausage-shaped but lumpy, Like a sausage but with cracks on the surface, Like a sausage or snake, smooth and soft, Soft blobs with clear-cut edges, Fluffy pieces with ragged edges, a mushy stool, and Watery, no solid pieces. Entirely Liquid. Types 1 and 2 indicate constipation. Types 3 and 4 are optimal, especially the latter, as these are the easiest to pass. Types 5-7 are associated with increasing tendency to diarrhea or urgency.

In one embodiment, the camera checks for the color of the stool as follows:

Brown: Human feces ordinarily has a light to dark brown coloration, which results from a combination of bile and bilirubin that is derived from dead red blood cells. Normally it is semisolid, with a mucus coating.

Yellow: Yellowing of feces can be caused by an infection known as Giardiasis, which derives its name from Giardia, an anaerobic flagellated protozoan parasite that can cause severe and communicable yellow diarrhea. Another cause of yellowing is a condition known as Gilbert's Syndrome. Yellow stool can also indicate that food is passing through the digestive tract relatively quickly. Yellow stool can be found in people with GERD gastroesophageal reflux disease.

Pale or Clay: Stool that is pale or grey may be caused by insufficient bile output due to conditions such as cholecystitis, gallstones, Giardia parasitic infection, hepatitis, chronic pancreatitis, or cirrhosis. Bile salts from the liver give stool its brownish color. If there is decreased bile output, stool is much lighter in color.

Black or Red: Feces can be black due to the presence of red blood cells that have been in the intestines long enough to be broken down by digestive enzymes. This is known as melena, and is typically due to bleeding in the upper digestive tract, such as from a bleeding peptic ulcer. Conditions that can also cause blood in the stool include hemorrhoids, anal fissures, diverticulitis, colon cancer, and ulcerative colitis. The same color change can be observed after consuming foods that contain a substantial proportion of animal blood, such as black pudding or tiétcanh. Black feces can also be caused by a number of medications, such as bismuth subsalicylate (the active ingredient in Pepto-Bismol), and dietary iron supplements, or foods such as beetroot, black liquorice, or blueberries. Hematochezia is similarly the passage of feces that are bright red due to the presence of undigested blood, either from lower in the digestive tract, or from a more active source in the upper digestive tract. Alcoholism can also provoke abnormalities in the path of blood throughout the body, including the passing of red-black stool.

Blue: Prussian blue, used in the treatment of radiation, cesium, and thallium poisoning, can turn the feces blue. Substantial consumption of products containing blue food dye, such as blue curacao or grape soda, can have the same effect.

Silver: A tarnished-silver or aluminum paint-like feces color characteristically results when biliary obstruction of any type (white stool) combines with gastrointestinal bleeding from any source (black stool). It can also suggest a carcinoma of the ampulla of Vater, which will result in gastrointestinal bleeding and biliary obstruction, resulting in silver stool.

Green: Feces can be green due to having large amounts of unprocessed bile in the digestive tract. This can occasionally be the result from eating liquorice candy, as it is typically made with anise oil rather than liquorice herb and is predominantly sugar. Excessive sugar consumption or a sensitivity to anise oil may cause loose, green stools.

Purple: Purple feces is a symptom of porphyria.

In another embodiment, an electronic nose is used to detect feces possess physiological odor, which can vary according to diet (especially the amount of meat protein e.g., methionine and health status. The odor of human feces is suggested to be made up from the following odorant volatiles:
Methyl sulfides: methylmercaptan/methanethiol (MM), dimethyl sulfide (DMS), dimethyl trisulfide (DMTS), dimethyl disulfide (DMDS)
Benzopyrrole volatiles: indole, skatole
Hydrogen sulfide (H2S)

(H2S) is the most common volatile sulfur compound in feces. The odor of feces may be increased in association with various pathologies, including: Celiac disease, Crohn's disease, ulcerative colitis, chronic pancreatitis, cystic fibrosis, intestinal infection, Clostridium difficile infection, malabsorption, short bowel syndrome.

The system can also control odor through UV light or chemicals such as bismuth subsalicylate, chloryphyllyn, herbs such as rosemary, yucca schidigera, zinc acetate.

In other embodiments, the pH of the stool also may be measured. A stool culture is done to find out if bacteria may be causing an infection. Other stool analytics can be done to:
Help identify diseases of the digestive tract, liver, and pancreas. Certain enzymes (such as trypsin or elastase) may be evaluated in the stool to help determine how well the pancreas is functioning.
Help find the cause of symptoms affecting the digestive tract, including prolonged diarrhea, bloody diarrhea, an increased amount of gas, nausea, vomiting, loss of appetite, bloating, abdominal pain and cramping, and fever.
Screen for colon cancer by checking for hidden (occult) blood.
Look for parasites, such as pinworms or Giardia lamblia.
Look for the cause of an infection, such as bacteria, a fungus, or a virus.
Check for poor absorption of nutrients by the digestive tract (malabsorption syndrome).

The electronic nose can have a sensor array, composed of a plurality of sensors, disposed within a cavity of the excrement container, each sensor for measuring the different variety of compounds within the gas sample. The number of arrays is limited by power consumption design requirements. In a preferred embodiment, two identical sensor arrays are disposed within the first cavity. Using multiple identical sensor arrays provides at least the following benefits; 1) fault tolerance methods for increased reliability can be employed; 2) enables a more accurate measurement of the sample is possible through the use of sensor array averaging methods; and 3) various error correction algorithms can be implemented. Each of the at least one sensor arrays measures properties of the gas sample and produces an output, which is received by a CPU (central processing unit) or processor in signal communication with each of the at least one sensor arrays, the processor for receiving the output and controlling operation of the at least one sensor array. The plurality of sensors used in each of the at least one sensor arrays can be of low-cost, non-selective commercial type gas sensors. For example, a hybrid structure array with a plurality of MOS, and/or MOSFET, and/or CP, and/or SAW and/or QCM, VOC gas sensors can be utilized. Ideally, each of the at least one sensor arrays should be composed of at least four different gas target and/or sensor type gas sensors as well as one temperature sensor and one humidity sensor in order to increase compound selectivity and response. Many manufacturers use different sensing technologies that generate different responses. It has been shown that comparative methods using responses from more types of sensors provide better overall results. In a preferred embodiment, one sensor array is positioned on an upper wall of the first cavity, and a second sensor array is positioned on a lower wall of the first cavity. It should be noted that there are various techniques such as temperature modulation and compound filtering that can be applied to the sensors and the gas sample in order to generate many virtual sensors from only a small number of physical sensors within each of the at least one sensor arrays. Since sensor performance improves at higher temperatures, a second heater may be utilized to heat the first cavity. For each sensor, the temperature of MOS film affects the kinetics of the adsorption and reaction processes that take place within the sensor. Also, in the presence of multiple compounds, each will react preferentially as the temperature of the sensor varies. In the same way, the higher temperatures within the first cavity may impact compound separation from each gas sample and facilitate better selective response from the sensors. Since temperature impacts the measurements it is beneficial to be able to modulate and control the temperature of both the sensors and the first cavity itself. For this reason, additional heaters (not shown) may be associated with each sensor array.

The camera can have image processing capability to detect diarrhea, bloody diarrhea. Other sensors can be used to detect an increased amount of gas, nausea, vomiting, loss of appetite, bloating, abdominal pain and cramping, and fever. The fecal elastase test is another test of pancreas function. The test measures the levels of elastase, an enzyme found in fluids produced by the pancreas. Elastase digests (breaks down) proteins. A fecal occult blood test can be used to diagnose many conditions that cause bleeding in the gastrointestinal system including colorectal cancer or stomach cancer. Parasitic diseases such as ascariasis, hookworm, strongyloidiasis and whipworm can be diagnosed by examining stools under a microscope for the presence of worm larvae or eggs. Some bacterial diseases can be detected with a stool culture. Toxins from bacteria such as Clostridium difficile can also be identified. Viruses such as rotavirus can also be found in stools. A fecal pH test may be used determine lactose intolerance or the presence of an infection. Steatorrhea can be diagnosed using a Fecal fat test that checks for the malabsorption of fat. Faecalelastase levels are becoming the mainstay of pancreatitis diagnosis

One test checks for pinworms, a type of roundworm. The roundworms are classified as parasites with microscopic eggs. Adults measure anywhere from five to ten centimeters. A camera is used to detect eggs and moving worms.

Another test detects colon cancer. Over 100,000 persons per year in the United States are afflicted with cancer of the colon and rectum. When the number of colon/rectal cancers occurring each year is combined with the number of cancers occurring in other digestive organs, including the esophagus and stomach, such cancers of the digestive system account for more occurrences of cancer than any other single form of the disease. Contrary to many other forms of cancer, early diagnosis and treatment of digestive tract cancer does result in a cure rate of 80% to 90%. If, however, the disease is not detected until the later stages, the cure rate drops significantly. Thus, early detection of the disease is important to successful treatment of digestive tract cancer. Most, but not all, cancers of the digestive tract bleed to a certain extent. This blood is deposited on and in fecal matter excreted from the digestive system. The presence of blood in fecal matter is not normally detected, however, until gross bleeding, that is, blood visible to the naked eye, occurs. Gross bleeding, however, is symptomatic of advanced cancers. Digestive tract cancers in the early stages, including pre-cancerous polyps, also tend to bleed, giving rise to occult (hidden) blood in the fecal matter. Other pathological conditions, such as Crohn's disease and diverticulitis, can also give rise to the presence of occult blood in the fecal matter.

Certain embodiments include diagnostic capability such as those for colorectal screening which save lives as a result. The embedded diagnostic in these embodiments provides a private and convenient means for preliminarily detecting fecal blood. Upon detecting blood, individuals are more likely to consult a health care physician for a colorectal screening. The test material is formed from biodegradable material or material that easily disintegrates in water so that the kit may be toilet disposed without exposing individuals to infectious micro-organisms.

One test that can be done is disclosed in Pagano U.S. Pat. No. 3,996,006, which is incorporated herein by reference in its entirety. In general, the Pagano test employs an absorbent paper impregnated with a guaiac reagent and encased in a special test slide having openable flaps on both sides of the test slide. A sample of fecal matter contacts the guaiac impregnated paper and a nonaqueous developing solution is applied to the guaiac impregnated paper. If occult blood is present in the fecal matter on the opposite side of the paper, the guaiac reaction will dye the paper blue, providing a positive indication of the presence of blood in the fecal matter.

In another occult blood test embodiment, the stool is mixed with a compound which, when present in an aqueous solution with at least one of blood, blood fractions, blood components and hemoglobin, results in a chemiluminescence. In further embodiments, the compound undergoes a reaction in aqueous solution which is catalyzed by at least one of blood, blood fractions, blood components and hemoglobin. In further embodiments, the reaction is catalyzed by the hem iron of hemoglobin. The system includes a luminescent, preferably dry luminol (C8H7N3O2), which may be packaged and contained in a container 22. Some compounds related to luminol such as: Luminol, hemihydrate; Luminol, Na salt; Luminol, HCL; isoluminol; isoluminol, monohydrate; and isoluminol ABEI, to name some examples, may be more or less suitable. Luminol may be synthesized using known means beginning from 3-nitrophthalic acid. First, hydrazine (N2H4) is heated with the 3-nitrophthalic acid in a high-boiling solvent such as triethylene glycol. Nitrophthalhydrazide is formed by a condensation reaction. Reduction of the nitro group on the Nitrophthalhydrazide yields luminol. To exhibit its luminescence, an amount of water (oxidant) sufficient to produce a mixture of the luminescent and sample is added. The lid may then be placed on the open end of the container and the contents swirled, shaken, or otherwise sufficiently mixed to thoroughly mix the aqueous solution with the sample. In one embodiment, the chemiluminescent compound undergoes a light-producing reaction which involves, as a reactant or catalyst, blood or blood components or products. In another embodiment, the chemiluminescent compound is luminol or a related compound, such as the examples listed above, which undergoes a luminescence-producing reaction in the container which is catalyzed by blood components, particularly the iron component of whole hemoglobin. In the presence of iron, which is found in the hemoglobin of blood, and which functions as a catalyst, the luminol will luminesce.

Yet other non-invasive diagnostic methods involve assaying stool samples for the presence of fecal occult blood or for elevated levels of carcinoembryonic antigen, both of which are suggestive of the presence of colorectal cancer. Additionally, techniques for detecting the presence of a range of DNA mutations or alterations associated with and indicative of the presence of colorectal cancer can be used. The presence of such mutations can be detected in DNA found in stool samples during the early stages of colorectal cancer. As cells and cellular debris are shed from colonic epithelial cells onto forming stool in a longitudinal “stripe” of material along the length of the stool, the system can take a representative sample in order to ensure that the sample will contain any cells or cellular debris that was shed into the stool as it passed through the colon. Accordingly, the system obtains a representative (e.g. a cross-section or circumferential surface) portion of stool voided by a patient, and performing an assay to detect in the sample the presence of cells or cellular debris shed from epithelial cells lining the colon that may be indicative of cancer or precancer. Most often, such cells will be derived from a polyp or a cancerous or precancerous lesion at a discrete location along the colon. For purposes of the present invention, a precancerous lesion comprises precancerous cells, and precancerous cells are cells that have a mutation that is associated with cancer and which renders such cells susceptible to becoming cancerous. A cross-sectional sample is a sample that contains at least a circumferential surface of the stool (or portion of a stool comprising an entire cross-sectional portion), as, for example, in a coronal section or a sagittal section. A sample comprising the surface layer of a stool (or of a cross-section of a stool) also contains at least a circumferential surface of the stool. Both cross-sections and circumferential surfaces comprise longitudinal stripes of sloughed colonic epithelium, and are therefore representative samples.

The housing (and the urine collector) can be cleaned with a UV light cleaning accessory. In one embodiment, “ultraviolet light” or simply “ultraviolet (UV)” is applied. UV is the electromagnetic radiation emitted from the region of the spectrum lying beyond the visible light and before x-rays. The upper wavelength limit is 400 nanometers (1 nm=10-g meter) and the lower wavelength limit is 100 nm, below which radiation ionizes virtually all molecules. The region between 400 and 190 nm has been divided into three regions: NEAR-ultraviolet radiation or UV-A can be considered to lie in the wavelength range 320-400 nm. The long wavelength limit represents the beginning of the visible spectrum, while the short wavelength limit corresponds roughly to the point below which proteins and genetic material begin to absorb significantly. Below this region is the MID-UV region or UV-B (290320 nm), where proteins and genetic material begin to absorb and where sunburn and skin cancer are most effectively produced. (UV radiation present in sunlight at the surface of the earth at noon in clear weather includes both the NEAR-UV and the MID-UV regions.) FAR-UV (UV-C) wavelengths range from 200-290 nm, and because of their strong absorption by genetic material, are highly destructive to biological matter. These wavelengths are almost all absorbed by the ozone in the stratosphere. The wavelength of ultraviolet light produced by the UV lamps which are used for the disinfection of water is 254 nm, which is in the FAR-UV or UV-C range.

The narrow band of UV light lying between the wavelengths of 200 and 300 nm has often been called the germicidal region because UV light in this region is lethal to microorganisms including: bacteria, protozoa, viruses, molds, yeasts, fungi, nematode eggs and algae. The most destructive wavelength is 260 nm which is very close to the wavelength of 254 nm produced by germicidal lamps. UV light's ability to kill the fecal coliform bacteria, Escherichia coli, is directly related to the ability of its genetic material (i.e. nucleic acid) to absorb UV light. UV light causes molecular rearrangements in the genetic material of microorganisms and this prevents them from reproducing. Most microorganisms have relatively short life cycles and therefore depend on rapid reproduction to sustain and grow their population. Therefore, if a microorganism cannot reproduce then it is considered to be dead. Normally when DNA replicates, the Thymine (T) must join the Adenine (A), and the Cytosine (C) must join with Guanine (G). When DNA is exposed to Ultraviolet Light at a wavelength of 254 nm, an error occurs in the replication process. The Thymine forms a dimer, that is, a double bond between the Thymine molecules. This error prevents the pathogen from reproducing properly and so eventually it dies off.

One embodiment is an exemplary airbag with a cartridge that activates when the accelerometer detects that the user is falling and needs cushioning. While only one set is shown, it is understood that as many sets can be used as desired. For example, four sets can be spaced apart on the front, back, and sides of the user to provide 360 degree protection. A sensor such as an angle change sensor, an altitude sensor, a G-force decrease sensor or other sensor recognizes a characteristic change that accompanies a fall. The processor actuates a compressed air (or other gas) chamber, and an air bag to each set. A release valve can be actuated for compressed air chamber to rapidly release its contents to air bag for full deployment. On/off switch may be utilized to deactivate the module so that a wearer may change any characteristic without setting off the air bags. In other words, the device can be turned on and off as desired, e. g., a motorcyclist can turn it on when embarking and shut it off when disembarking.

In other embodiments, the air bag can be in the form of a vest device. The vest device has a front bag and a supply and control module well as a rear bag and a supply and control module. Once a fall is detected, the air bags are deployed in time to create a soft fall. In one embodiment, the front air bag blows up to support the chin and neck but not to shut off breathing, while rear air bag extends up the back of the neck and the back of the head to protect both the neck and the back of the head. In another embodiment, a set of separate jacket and pants present invention air bag can be used. Here jacket has a hood with a head back air bag system (this system includes at least one air bag and at least one module), arms with air bags. There is also a front chest bag. Pants includes hip units, such as left hip air bag, as well as leg units, such as leg air bag. The jacket and pants function in a manner the same as described above. In other embodiments, attachment means such as belt strap and latch and corresponding belt strap and buckle are exemplary and can be used to attach to a torso, back or buttocks. Alternatively, it could be in the form of a belt and attached to a waist. The shock buffering protection will be activated immediately upon a fall detection to release gas such as CO2 gas into the neck, chest, body, back and hip airbags to inflate them in a brief time such as 0.5 second to reduce the impact of the fall.

In one embodiment, the printed flexible fall detector can be smart clothing with a microcontroller with accelerometers, gyroscopes, and magnetometers. Optionally, the fall detector can have a vertical detector to detect if the patient is on the ground. In other embodiment, the detection of height can be done using an accelerometer, where the accelerometer will be dropped in one translation down from the height to the earth. For rotational, the accelerometer will drop, but it will also have a spin to it, and a rotation. With linear, rotational and projectile falls, the system can determine the height of the fall by sampling by knowing the rate that the accelerometer is sampled by the microcontroller, the time that an object starts to fall, and the time that impact occurs. This gives a difference equal to the time of the fall. This information can be taken with an equation to determine the height of the fall. The fall detector can be a tag in a standalone mode that actuates the gas generator using an electrically actuated pin that punctures the gas canister. The fall detector can also be a portable consumer device such as a smart phone. Either can work alone, and for improved detection accuracy, the fall detector can employ software on both the tag and the smart phone. In one embodiment, the application (App) can be downloaded from a store to the phone. The App can be put into “test mode” where the user can see which motions trigger the “alarm” and which don't. The app will have some “thresholds” that will need to be set to “optimize” performance. If a fall is detected and the sensors detect that the user cannot get up within a predetermined time, the phone can make outgoing calls to a sequence of emergency contacts, including a call center, family telephone number, caregiver telephone number, and other helper's telephone number if a fall is detected. Voice, text or email messages can be sent. The user will be able to override an emergency phone call by manually cancelling the call. Text messages will not be able to be cancelled.

One exemplary system provides for monitoring urination and/or defecation and reporting the event to staff or a caregiver for assistance. Embodiments provide information regarding the nature and volume of exudate associated with a wetness event and more particularly, the volume of individual events in a sequence of events occurring during the wearing of an absorbent pad. This information is useful to be able to determine the frequency, type and severity of each incontinence episode suffered by an individual and developing an incontinence profile in order to prescribe a suitable treatment or management plan for the individual's incontinence. The system can then determine when the total amount of exudate absorbed by an absorbent pad is approaching or has reached the limit of the pad's absorbent capacity and whether changing of the pad is required. The system can determine whether an absorbent pad is likely to require changing without necessarily requiring manual periodic checking of the pad by staff in a care facility.

The system can work with the sensors discussed above. Alternatively, for a conventional diaper, the system can work with an exudate sensor that includes a pad body, one or more wings formed at two sides of the pad body, a top layer, a cover layer and multiple capacitive or resistive wetness sensors in the cover layer. In one embodiment, in lieu of the resistive/capacitive sensors, a humidity sensor detects humidity around the diaper and determines the urine volume in the diaper. A separate capacitive or resistive fecal sensor is placed so that it is underneath the anus during use to detect the extrusion of fecal matters. In another embodiment, the processor receives the wetness information from the capacitive or resistive sensors and estimates the volume of urine received by the diaper so far and if capacity has been reach, signals the staff or caregiver to change the diaper and clean the patient. The volume estimation is done by detecting which grid had a short and the length of the short. The system knows the area of each grid, and by integrating the areas that had shorts caused by the resistive elements in the grid array, the system can estimate the volume of urine secreted. If the volume exceeds a predetermined volume which is greater than a minor leak, the system alerts caregivers to change clothing and clean the user. The processor can compare the estimated volume with a pre-defined threshold level. If the estimated volume is less than the threshold, the processor continues to monitor the sensor signals. If the estimated volume exceeds the threshold amount, then the processor sends an alert to a caregiver (carer). Once a carer is alerted, the carer attends to the resident and may choose to change the absorbent article and the processor detects that the sensor has been disconnected from the system and resets the sensor data. The threshold volume used by the processor to alert a carer may be a “qualifying amount” e.g. indicated as small, medium or large or a quantifying amount being a pre-defined volume e.g. 50 ml.

Preferably, the processor may also execute an algorithm to compare the estimated volume with a known estimated capacity of the diaper to give carers an indication of when the diaper is likely to become saturated with exudate so that it can be changed before a saturating wetness event occurs and the patient is made to feel uncomfortable by excess wetness.

The processor may also monitor the total amount of accumulated moisture in a series of wetness events in a single absorbent article and provide an indication to a carer as to when the absorbent capacity of the garment has been or is likely to be reached, to prompt the carer to change the garment for the patient's comfort and wellbeing.

Users may enter data, including patient specific demographic data such as gender, age, height and weight via user interface. Other entered data may include medical data, i.e. medication, amount of fluid and food intake, details of known conditions, recent surgeries, years in assisted care, years wearing an incontinence garment, continence function if known, and mental condition.

The processor may be incorporated into a central monitoring station such as a nurse's station. The processor may also integrate with or be incorporated into existing nurse call and remote patient monitoring systems controlled at the nurse's station. The processor may also be integrated with other care management systems for streamlining access to non-sensor related data contained within other care management systems such as, for example, fluid and food intake, patient relocation, showering, toileting, surgeries etc.

User interface may also include a transmitter which sends alerts to communication devices such as pagers or nurse phones carried by carers to indicate that there has been a wetness event, or that one is due to occur, or that physical inspection of the patient is required or due. In addition to the detection of wetness events which are estimated to exceed a threshold amount, these conditions warranting physical inspection may include when exudate is fecal in nature or when sensors detect blood, a parasite or a biological or chemical marker in the urine or faeces.

In one embodiment, observation data is used, along with a log of the sensor signals received at the input, to identify patterns in the patent's continence activity. The processor derives automatically, using an algorithm employing another mathematical model, a continence care plan based on the pattern, i.e. frequency and repetition of monitored events. The care plan includes a voiding or toileting schedule which statistically predicts wetness events based on the observed pattern. This is used by carers to plan the regularity (e.g. times of day) that a patient is to be manually checked for wetness and/or assisted with toileting and to plan when to empty the bladder or bowel, prior to periods in which a patient is known to have a pattern of incontinence events. Normal care of the patient can then take place without the need to continually monitor using a sensor.

The voiding schedule anticipates when a wetness event is statistically likely to occur and this can be used to automatically generate an audible and/or visible alert for a carer (e.g. presented on a screen of the user interface 108 or transmitted to a pager or the like) to attend to the patient by assisting with manual toileting or to change the patient's incontinence garment.

It is recommended that the toileting/voiding schedule is re-evaluated periodically (step 310) to maintain its accuracy, in keeping with changes in the patient's continence patterns. Re-evaluation may take place for example every 3, 6 or 12 months, or whenever actual wetness events do not correspond well with those anticipated by the voiding schedule.

In another use of the invention, the moisture monitoring system includes a log for recording wetness events detected by sensors including the volume, time and nature (urinary and/or fecal) of each event. These data are used to produce a bladder diary. These data may also be combined with details entered e.g. at the user interface 108 which relate to food and fluid intake (amount, kind and time), toileting and also any particular activities that the patient has undertaken.

The log may manifest in a memory device in communication or integrated with the processor. The processor may be located centrally and receive sensor signals relating indicative of wetness of a number of absorbent articles worn by different patients. Alternatively, there may be a pre-processor executing the algorithm located near the sensor, on the absorbent article. That is, the sensor and the part of the processor performing the analysis may be a provided together with the sensor. In such arrangement, the pre-processor may also incorporate a transmitter for transmitting data from the pre-processor to e.g. a central monitoring system which may include a display.

One embodiment includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the patient body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies.

In a tetrapolar implementation, two electrodes on the wrist watch or wrist band are used to apply AC or DC constant current into the body or body segment. The voltage signal from the surface of the body is measured in terms of impedance using the same or an additional two electrodes on the watch or wrist band. In a bipolar implementation, one electrode on the wrist watch or wrist band is used to apply AC or DC constant current into the body or body segment. The voltage signal from the surface of the body is measured in terms of impedance using the same or an alternative electrode on the watch or wrist band. The system may include a BI patch that wirelessly communicates BI information with the wrist watch. Other patches 1400 can be used to collect other medical information or vital parameter and communicate with the wrist watch or base station or the information could be relayed through each wireless node or appliance to reach a destination appliance such as the base station, for example. The system can also include a head-cap 1402 that allows a number of EEG probes access to the brain electrical activities, EKG probes to measure cranial EKG activity, as well as BI probes to determine cranial fluid presence indicative of a stroke. As will be discussed below, the EEG probes allow the system to determine cognitive status of the patient to determine whether a stroke had just occurred, the EKG and the BI probes provide information on the stroke to enable timely treatment to minimize loss of functionality to the patient if treatment is delayed.

Bipolar or tetrapolar electrode systems can be used in the BI instruments. Of these, the tetrapolar system provides a uniform current density distribution in the body segment and measures impedance with less electrode interface artifact and impedance errors. In the tetrapolar system, a pair of surface electrodes (11, 12) is used as current electrodes to introduce a low intensity constant current at high frequency into the body. A pair of electrodes (E1, E2) measures changes accompanying physiological events. Voltage measured across E1-E2 is directly proportional to the segment electrical impedance of the human subject. Circular flat electrodes as well as band type electrodes can be used. In one embodiment, the electrodes are in direct contact with the skin surface. In other embodiments, the voltage measurements may employ one or more contactless, voltage sensitive electrodes such as inductively orcapacitively coupled electrodes. The current application and the voltage measurement electrodes in these embodiments can be the same, adjacent to one another, or at significantly different locations. The electrode(s) can apply current levels from 20 uA to 10 mA rms at a frequency range of 20-100 KHz. A constant current source and high input impedance circuit is used in conjunction with the tetrapolar electrode configuration to avoid the contact pressure effects at the electrode-skin interface.

The BI sensor can be a Series Model which assumes that there is one conductive path and that the body consists of a series of resistors. An electrical current, injected at a single frequency, is used to measure whole body impedance (i.e., wrist to ankle) for the purpose of estimating total body water and fat free mass. Alternatively, the BI instrument can be a Parallel BI Model In this model of impedance, the resistors and capacitors are oriented both in series and in parallel in the human body. Whole body BI can be used to estimate TBW and FFM in healthy subjects or to estimate intracellular water (ICW) and body cell mass (BCM). High-low BI can be used to estimate extracellular water (ECW) and total body water (TBW). Multi-frequency BI can be used to estimate ECW, ICW, and TBW; to monitor changes in the ECW/BCM and ECW/TBW ratios in clinical populations. The instrument can also be a Segmental BI Model and can be used in the evaluation of regional fluid changes and in monitoring extra cellular water in patients with abnormal fluid distribution, such as those undergoing hemodialysis. Segmental BI can be used to measure fluid distribution or regional fluid accumulation in clinical populations. Upper-body and Lower-body BI can be used to estimate percentage BF in healthy subjects with normal hydration status and fluid distribution. The BI sensor can be used to detect acute dehydration, pulmonary edema (caused by mitral stenosis or left ventricular failure or congestive heart failure, among others), or hyperhydration cause by kidney dialysis, for example. In one embodiment, the system determines the impedance of skin and subcutaneous adipose tissue using tetrapolar and bipolar impedance measurements. In the bipolar arrangement the inner electrodes act both as the electrodes that send the current (outer electrodes in the tetrapolar arrangement) and as receiving electrodes. If the outer two electrodes (electrodes sending current) are superimposed onto the inner electrodes (receiving electrodes) then a bipolar BIA arrangement exists with the same electrodes acting as receiving and sending electrodes. The difference in impedance measurements between the tetrapolar and bipolar arrangement reflects the impedance of skin and subcutaneous fat. The difference between the two impedance measurements represents the combined impedance of skin and subcutaneous tissue at one or more sites. The system determines the resistivities of skin and subcutaneous adipose tissue, and then calculates the skinfold thickness (mainly due to adipose tissue).

Various BI analysis methods can be used in a variety of clinical applications such as to estimate body composition, to determine total body water, to assess compartmentalization of body fluids, to provide cardiac monitoring, measure blood flow, dehydration, blood loss, wound monitoring, ulcer detection and deep vein thrombosis. Other uses for the BI sensor includes detecting and/or monitoring hypovolemia, hemorrhage or blood loss. The impedance measurements can be made sequentially over a period of in time; and the system can determine whether the subject is externally or internally bleeding based on a change in measured impedance. The watch can also report temperature, heat flux, vasodilation and blood pressure along with the BI information.

In one embodiment, the BI system monitors cardiac function using impedance cardiography (ICG) technique. ICG provides a single impedance tracing, from which parameters related to the pump function of the heart, such as cardiac output (CO), are estimated. ICG measures the beat-to-beat changes of thoracic bioimpedance via four dual sensors applied on the neck and thorax in order to calculate stroke volume (SV). By using the resistivity p of blood and the length L of the chest, the impedance change ΔZ and base impedance (Zo) to the volume change ΔV of the tissue under measurement can be derived as follows:

$$Δ V = ρ \frac{{L}^{2}}{{Z}_{0}^{2}} Δ Z$$

In one embodiment, SV is determined as a function of the first derivative of the impedance waveform (dZ/dtmax) and the left ventricular ejection time (LVET)

$$SV = ρ \frac{{L}^{2}}{{Z}_{0}^{2}} {( \frac{dZ}{dt} )}_{max} LVET$$

In one embodiment, L is approximated to be 17% of the patient's height (H) to yield the following:

$$SV = ( \frac{{( 0.17 H )}^{3}}{4.2} ) \frac{{( \frac{dZ}{dt} )}_{max}}{{Z}_{0}} LVET$$

In another embodiment or the actual weight divided by the ideal weight is used:

$$SV = δ \times ( \frac{{( 0.17 H )}^{3}}{4.2} ) \frac{{( \frac{dZ}{dt} )}_{max}}{{Z}_{0}} LVET$$

The impedance cardiographic embodiment allows hemodynamic assessment to be regularly monitored to avoid the occurrence of an acute cardiac episode. The system provides an accurate, noninvasive measurement of cardiac output (CO) monitoring so that ill and surgical patients undergoing major operations such as coronary artery bypass graft (CABG) would benefit. In addition, many patients with chronic and comorbid diseases that ultimately lead to the need for major operations and other costly interventions might benefit from more routine monitoring of CO and its dependent parameters such as systemic vascular resistance (SVR).

Once SV has been determined, CO can be determined according to the following expression:

CO=SV*HR,

where HR=heart rate

CO can be determined for every heart-beat. Thus, the system can determine SV and CO on a beat-to-beat basis.

In one embodiment to monitor heart failure, an array of BI sensors are place in proximity to the heart. The array of BI sensors detect the presence or absence, or rate of change, or body fluids proximal to the heart. The BI sensors can be supplemented by the EKG sensors. A normal, healthy, heart beats at a regular rate. Irregular heart beats, known as cardiac arrhythmia, on the other hand, may characterize an unhealthy condition. Another unhealthy condition is known as congestive heart failure (“CHF”). CHF, also known as heart failure, is a condition where the heart has inadequate capacity to pump sufficient blood to meet metabolic demand. CHF may be caused by a variety of sources, including, coronary artery disease, myocardial infarction, high blood pressure, heart valve disease, cardiomyopathy, congenital heart disease, endocarditis, myocarditis, and others. Unhealthy heart conditions may be treated using a cardiac rhythm management (CRM) system. Examples of CRM systems, or pulse generator systems, include defibrillators (including implantable cardioverter defibrillator), pacemakers and other cardiac resynchronization devices.

In one implementation, BIA measurements can be made using an array of bipolar or tetrapolar electrodes that deliver a constant alternating current at 50 KHz frequency. Whole body measurements can be done using standard right-sided. The ability of any biological tissue to resist a constant electric current depends on the relative proportions of water and electrolytes it contains, and is called resistivity (in Ohms/cm 3). The measuring of bioimpedance to assess congestive heart failure employs the different bio-electric properties of blood and lung tissue to permit separate assessment of: (a) systemic venous congestion via a low frequency or direct current resistance measurement of the current path through the right ventricle, right atrium, superior vena cava, and subclavian vein, or by computing the real component of impedance at a high frequency, and (b) pulmonary congestion via a high frequency measurement of capacitive impedance of the lung. The resistance is impedance measured using direct current or alternating current (AC) which can flow through capacitors.

In one embodiment, a belt is worn by the patient with a plurality of BI probes positioned around the belt perimeter. The output of the tetrapolar probes is processed using a second-order Newton-Raphson method to estimate the left and right-lung resistivity values in the thoracic geometry. The locations of the electrodes are marked. During the measurements procedure, the belt is worn around the patient's thorax while sitting, and the reference electrode is attached to his waist. The data is collected during tidal respiration to minimize lung resistivity changes due to breathing, and lasts approximately one minute. The process is repeated periodically and the impedance trend is analyzed to detect CHF. Upon detection, the system provides vital parameters to a call center and the call center can refer to a physician for consultation or can call 911 for assistance.

In one embodiment, an array of noninvasive thoracic electrical bioimpedance monitoring probes can be used alone or in conjunction with other techniques such as impedance cardiography (ICG) for early comprehensive cardiovascular assessment and trending of acute trauma victims. This embodiment provides early, continuous cardiovascular assessment to help identify patients whose injuries were so severe that they were not likely to survive. This included severe blood and/or fluid volume deficits induced by trauma, which did not respond readily to expeditious volume resuscitation and vasopressor therapy. One exemplary system monitors cardiorespiratory variables that served as statistically significant measures of treatment outcomes: Qt, BP, pulse oximetry, and transcutaneous Pot (Ptco2). A high Qt may not be sustainable in the presence of hypovolemia, acute anemia, pre-existing impaired cardiac function, acute myocardial injury, or coronary ischemia. Thus a fall in Ptco2 could also be interpreted as too high a metabolic demand for a patient's cardiovascular reserve. Too high a metabolic demand may compromise other critical organs. Acute lung injury from hypotension, blunt trauma, and massive fluid resuscitation can drastically reduce respiratory reserve.

One embodiment that measures thoracic impedance (a resistive or reactive impedance associated with at least a portion of a thorax of a living organism). The thoracic impedance signal is influenced by the patient's thoracic intravascular fluid tension, heart beat, and breathing (also referred to as “respiration” or “ventilation”). A “de” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject patient's thoracic fluid tension, and is therefore influenced by intravascular fluid shifts to and away from the thorax. Higher frequency components of the thoracic impedance signal are influenced by the patient's breathing (e.g., approximately between 0.05 Hz and 2.0 Hz inclusive) and heartbeat (e.g., approximately between 0.5 Hz and 10 Hz inclusive). A low intravascular fluid tension in the thorax (“thoracic hypotension”) may result from changes in posture. For example, in a person who has been in a recumbent position for some time, approximately ⅓ of the blood volume is in the thorax. When that person then sits upright, approximately ⅓ of the blood that was in the thorax migrates to the lower body. This increases thoracic impedance. Approximately 90% of this fluid shift takes place within 2 to 3 minutes after the person sits upright.

The accelerometer can be used to provide reproducible measurements. Body activity will increase cardiac output and also change the amount of blood in the systemic venous system or lungs. Measurements of congestion may be most reproducible when body activity is at a minimum and the patient is at rest. The use of an accelerometer allows one to sense both body position and body activity. Comparative measurements over time may best be taken under reproducible conditions of body position and activity. Ideally, measurements for the upright position should be compared as among themselves. Likewise measurements in the supine, prone, left lateral decubitus and right lateral decubitus should be compared as among themselves. Other variables can be used to permit reproducible measurements, i.e. variations of the cardiac cycle and variations in the respiratory cycle. The ventricles are at their most compliant during diastole. The end of the diastolic period is marked by the QRS on the electrocardiographic means (EKG) for monitoring the cardiac cycle. The second variable is respiratory variation in impedance, which is used to monitor respiratory rate and volume. As the lungs fill with air during inspiration, impedance increases, and during expiration, impedance decreases. Impedance can be measured during expiration to minimize the effect of breathing on central systemic venous volume. While respiration and CHF both cause variations in impedance, the rates and magnitudes of the impedance variation are different enough to separate out the respiratory variations which have a frequency of about 8 to 60 cycles per minute and congestion changes which take at least several minutes to hours or even days to occur. Also, the magnitude of impedance change is likely to be much greater for congestive changes than for normal respiratory variation. Thus, the system can detect congestive heart failure (CHF) in early stages and alert a patient to prevent disabling and even lethal episodes of CHF. Early treatment can avert progression of the disorder to a dangerous stage.

In an embodiment to monitor wounds such as diabetic related wounds, the conductivity of a region of the patient with a wound or is susceptible to wound formation is monitored by the system. The system determines healing wounds if the impedance and reactance of the wound region increases as the skin region becomes dry. The system detects infected, open, interrupted healing, or draining wounds through lower regional electric impedances. In yet another embodiment, the bioimpedance sensor can be used to determine body fat. In one embodiment, the BI system determines Total Body Water (TBW) which is an estimate of total hydration level, including intracellular and extracellular water; Intracellular Water (ICW) which is an estimate of the water in active tissue and as a percent of a normal range (near 60% of TBW); Extracellular Water (ECW) which is water in tissues and plasma and as a percent of a normal range (near 40% of TBW); Body Cell Mass (BCM) which is an estimate of total pounds/kg of all active cells; Extracellular Tissue (ECT)/Extracellular Mass (ECM) which is an estimate of the mass of all other non-muscle inactive tissues including ligaments, bone and ECW; Fat Free Mass (FFM)/Lean Body Mass (LBM) which is an estimate of the entire mass that is not fat. It should be available in pounds/kg and may be presented as a percent with a normal range; Fat Mass (FM) which is an estimate of pounds/kg of body fat and percentage body fat; and Phase Angle (PA) which is associated with both nutrition and physical fitness.

Additional sensors such as thermocouples or thermistors and/or heat flux sensors can also be provided to provide measured values useful in analysis. In general, skin surface temperature will change with changes in blood flow in the vicinity of the skin surface of an organism. Such changes in blood flow can occur for a number of reasons, including thermal regulation, conservation of blood volume, and hormonal changes. In one implementation, skin surface measurements of temperature or heat flux are made in conjunction with hydration monitoring so that such changes in blood flow can be detected and appropriately treated.

In one embodiment, the patch includes a sound transducer such as a microphone or a piezoelectric transducer to pick up sound produced by bones or joints during movement. If bone surfaces are rough and poorly lubricated, as in an arthritic knee, they will move unevenly against each other, producing a high-frequency, scratching sound. The high-frequency sound from joints is picked up by wide-band acoustic sensor(s) or microphone(s) on a patient's body such as the knee. As the patient flexes and extends their knee, the sensors measure the sound frequency emitted by the knee and correlate the sound to monitor osteoarthritis, for example.

In another embodiment, the patch includes a Galvanic Skin Response (GSR) sensor. In this sensor, a small current is passed through one of the electrodes into the user's body such as the fingers and the CPU calculates how long it takes for a capacitor to fill up. The length of time the capacitor takes to fill up allows us to calculate the skin resistance: a short time means low resistance while a long time means high resistance. The GSR reflects sweat gland activity and changes in the sympathetic nervous system and measurement variables. Measured from the palm or fingertips, there are changes in the relative conductance of a small electrical current between the electrodes. The activity of the sweat glands in response to sympathetic nervous stimulation (Increased sympathetic activation) results in an increase in the level of conductance. Fear, anger, startle response, orienting response and sexual feelings are all among the emotions which may produce similar GSR responses.

In yet another embodiment, measurement of lung function such as peak expiratory flow readings is done though a sensor such as Wright's peak flow meter. In another embodiment, a respiratory estimator is provided that avoids the inconvenience of having the patient breathing through the flow sensor. In the respiratory estimator embodiment, heart period data from EKG/ECG is used to extract respiratory detection features. The heart period data is transformed into time-frequency distribution by applying a time-frequency transformation such as short-term Fourier transformation (STFT). Other possible methods are, for example, complex demodulation and wavelet transformation. Next, one or more respiratory detection features may be determined by setting up amplitude modulation of time-frequency plane, among others. The respiratory recognizer first generates a math model that correlates the respiratory detection features with the actual flow readings. The math model can be adaptive based on pre-determined data and on the combination of different features to provide a single estimate of the respiration. The estimator can be based on different mathematical functions, such as a curve fitting approach with linear or polynomical equations, and other types of neural network implementations, non-linear models, fuzzy systems, time series models, and other types of multivariate models capable of transferring and combining the information from several inputs into one estimate. Once the math model has been generated, the respirator estimator provides a real-time flow estimate by receiving EKG/ECG information and applying the information to the math model to compute the respiratory rate. Next, the computation of ventilation uses information on the tidal volume. An estimate of the tidal volume may be derived by utilizing different forms of information on the basis of the heart period signal. For example, the functional organization of the respiratory system has an impact in both respiratory period and tidal volume. Therefore, given the known relationships between the respiratory period and tidal volume during and transitions to different states, the information inherent in the heart period derived respiratory frequency may be used in providing values of tidal volume. In specific, the tidal volume contains inherent dynamics which may be, after modeling, applied to capture more closely the behavioral dynamics of the tidal volume. Moreover, it appears that the heart period signal, itself, is closely associated with tidal volume and may be therefore used to increase the reliability of deriving information on tidal volume. The accuracy of the tidal volume estimation may be further enhanced by using information on the subjects vital capacity (i.e., the maximal quantity of air that can be contained in the lungs during one breath). The information on vital capacity, as based on physiological measurement or on estimates derived from body measures such as height and weight, may be helpful in estimating tidal volume, since it is likely to reduce the effects of individual differences on the estimated tidal volume. Using information on the vital capacity, the mathematical model may first give values on the percentage of lung capacity in use, which may be then transformed to liters per breath. T he optimizing of tidal volume estimation can based on, for example, least squares or other type of fit between the features and actual tidal volume. The minute ventilation may be derived by multiplying respiratory rate (breaths/min) with tidal volume (liters/breath).

In another embodiment, inductive plethysmography can be used to measure a cross-sectional area of the body by determining the self-inductance of a flexible conductor closely encircling the area to be measured. Since the inductance of a substantially planar conductive loop is well known to vary as, inter alia, the cross-sectional area of the loop, a inductance measurement may be converted into a plethysmographic area determination. Varying loop inductance may be measured by techniques known in the art, such as, e.g., by connecting the loop as the inductance in a variable frequency LC oscillator, the frequency of the oscillator then varying with the cross-sectional area of the loop inductance varies. Oscillator frequency is converted into a digital value, which is then further processed to yield the physiological parameters of interest. Specifically, a flexible conductor measuring a cross-sectional area of the body is closely looped around the area of the body so that the inductance, and the changes in inductance, being measured results from magnetic flux through the cross-sectional area being measured. The inductance thus depends directly on the cross-sectional area being measured, and not indirectly on an area which changes as a result of the factors changing the measured cross-sectional area. Various physiological parameters of medical and research interest may be extracted from repetitive measurements of the areas of various cross-sections of the body. For example, pulmonary function parameters, such as respiration volumes and rates and apneas and their types, may be determined from measurements of, at least, a chest transverse cross-sectional area and also an abdominal transverse cross-sectional area. Cardiac parameters, such central venous pressure, left and right ventricular volumes waveforms, and aortic and carotid artery pressure waveforms, may be extracted from repetitive measurements of transverse cross-sectional areas of the neck and of the chest passing through the heart. Timing measurements can be obtained from concurrent ECG measurements, and less preferably from the carotid pulse signal present in the neck. From the cardiac-related signals, indications of ischemia may be obtained independently of any ECG changes. Ventricular wall ischemia is known to result in paradoxical wall motion during ventricular contraction (the ischemic segment paradoxically “balloons” outward instead of normally contracting inward). Such paradoxical wall motion, and thus indications of cardiac ischemia, may be extracted from chest transverse cross-section area measurements. Left or right ventricular ischemia may be distinguished where paradoxical motion is seen predominantly in left or right ventricular waveforms, respectively. For another example, observations of the onset of contraction in the left and right ventricles separately may be of use in providing feedback to bi-ventricular cardiac pacing devices. For a further example, pulse oximetry determines hemoglobin saturation by measuring the changing infrared optical properties of a finger. This signal may be disambiguated and combined with pulmonary data to yield improved information concerning lung function.

In one embodiment to monitor and predict stroke attack, a cranial bioimpedance sensor is applied to detect fluids in the brain. The brain tissue can be modeled as an electrical circuit where cells with the lipid bilayer act as capacitors and the intra and extra cellular fluids act as resistors. The opposition to the flow of the electrical current through the cellular fluids is resistance. The system takes 50-kHz single-frequency bioimpedance measurements reflecting the electrical conductivity of brain tissue. The opposition to the flow of the current by the capacitance of lipid bilayer is reactance. In this embodiment, microamps of current at 50 kHz are applied to the electrode system. In one implementation, the electrode system consists of a pair of coaxial electrodes each of which has a current electrode and a voltage sensing electrode. For the measurement of cerebral bioimpedance, one pair of gel current electrodes is placed on closed eyelids and the second pair of voltage electrodes is placed in the suboccipital region projecting towards the foramen magnum. The electrical current passes through the orbital fissures and brain tissue. The drop in voltage is detected by the suboccipital electrodes and then calculated by the processor to bioimpedance values. The bioimpedance value is used to detect brain edema, which is defined as an increase in the water content of cerebral tissue which then leads to an increase in overall brain mass. Two types of brain edema are vasogenic or cytotoxic. Vasogenic edema is a result of increased capillary permeability. Cytotoxic edema reflects the increase of brain water due to an osmotic imbalance between plasma and the brain extracellular fluid. Cerebral edema in brain swelling contributes to the increase in intracranial pressure and an early detection leads to timely stroke intervention.

In another example, a cranial bioimpedance tomography system contructs brain impedance maps from surface measurements using nonlinear optimization. A nonlinear optimization technique utilizing known and stored constraint values permits reconstruction of a wide range of conductivity values in the tissue. In the nonlinear system, a Jacobian Matrix is renewed for a plurality of iterations. The Jacobian Matrix describes changes in surface voltage that result from changes in conductivity. The Jacobian Matrix stores information relating to the pattern and position of measuring electrodes, and the geometry and conductivity distributions of measurements resulting in a normal case and in an abnormal case. The nonlinear estimation determines the maximum voltage difference in the normal and abnormal cases.

In one embodiment, an electrode array sensor can include impedance, bio-potential, or electromagnetic field tomography imaging of cranial tissue. The electrode array sensor can be a geometric array of discrete electrodes having an equally-spaced geometry of multiple nodes that are capable of functioning as sense and reference electrodes. In a typical tomography application the electrodes are equally-spaced in a circular configuration. Alternatively, the electrodes can have non-equal spacing and/or can be in rectangular or other configurations in one circuit or multiple circuits. Electrodes can be configured in concentric layers too. Points of extension form multiple nodes that are capable of functioning as an electrical reference. Data from the multiple reference points can be collected to generate a spectrographic composite for monitoring over time.

The patient's brain cell generates an electromagnetic field of positive or negative polarity, typically in the millivolt range. The sensor measures the electromagnetic field by detecting the difference in potential between one or more test electrodes and a reference electrode. The bio-potential sensor uses signal conditioners or processors to condition the potential signal. In one example, the test electrode and reference electrode are coupled to a signal conditioner/processor that includes a lowpass filter to remove undesired high frequency signal components. The electromagnetic field signal is typically a slowly varying DC voltage signal. The lowpass filter removes undesired alternating current components arising from static discharge, electromagnetic interference, and other sources.

In one embodiment, the impedance sensor has an electrode structure with annular concentric circles including a central electrode, an intermediate electrode and an outer electrode, all of which are connected to the skin. One electrode is a common electrode and supplies a low frequency signal between this common electrode and another of the three electrodes. An amplifier converts the resulting current into a voltage between the common electrode and another of the three electrodes. A switch switches between a first circuit using the intermediate electrode as the common electrode and a second circuit that uses the outer electrode as a common electrode. The sensor selects depth by controlling the extension of the electric field in the vicinity of the measuring electrodes using the control electrode between the measuring electrodes. The control electrode is actively driven with the same frequency as the measuring electrodes to a signal level taken from one of the measuring electrodes but multiplied by a complex number with real and imaginary parts controlled to attain a desired depth penetration. The controlling field functions in the manner of a field effect transistor in which ionic and polarization effects act upon tissue in the manner of a semiconductor material.

With multiple groups of electrodes and a capability to measure at a plurality of depths, the system can perform tomographic imaging or measurement, and/or object recognition. In one embodiment, a fast reconstruction technique is used to reduce computation load by utilizing prior information of normal and abnormal tissue conductivity characteristics to estimate tissue condition without requiring full computation of a non-linear inverse solution.

In another embodiment, the bioimpedance system can be used with electro-encephalograph (EEG) or ERP. Since this embodiment collects signals related to blood flow in the brain, collection can be concentrated in those regions of the brain surface corresponding to blood vessels of interest. A headcap with additional electrodes placed in proximity to regions of the brain surface fed by a blood vessel of interest, such as the medial cerebral artery enables targeted information from the regions of interest to be collected. The headcap can cover the region of the brain surface that is fed by the medial cerebral artery. Other embodiments of the headcap can concentrate electrodes on other regions of the brain surface, such as the region associated with the somatosensory motor cortex. In alternative embodiments, the headcap can cover the skull more completely. Further, such a headcap can include electrodes thoughout the cap while concentrating electrodes in a region of interest. Depending upon the particular application, arrays of 1-16 head electrodes may be used, as compared to the International 10/20 system of 19-21 head electrodes generally used in an EEG instrument.

In one implementation, each amplifier for each EEG channel is a high quality analog amplifier device. Full bandwidth and ultra-low noise amplification are obtained for each electrode. Low pass, high pass, hum notch filters, gain, un-block, calibration and electrode impedance check facilities are included in each amplifier. All 8 channels in one EEG amplifier unit have the same filter, gain, etc. settings. Noise figures of less than 0.1 uVr·m·s. are achieved at the input and optical coupling stages. These figures, coupled with good isolation/common mode rejection result in signal clarity. Nine high pass filter ranges include 0.01 Hz for readiness potential measurement, and 30 Hz for EMG measurement.

In one embodiment, stimulations to elicit EEG signals are used in two different modes, i.e., auditory clicks and electric pulses to the skin. The stimuli, although concurrent, are at different prime number frequencies to permit separation of different evoked potentials (EPs) and avoid interference. Such concurrent stimulations for EP permit a more rapid, and less costly, examination and provide the patient's responses more quickly. Power spectra of spontaneous EEG, waveshapes of Averaged Evoked Potentials, and extracted measures, such as frequency specific power ratios, can be transmitted to a remote receiver. The latencies of successive EP peaks of the patient may be compared to those of a normal group by use of a normative template. To test for ischemic stroke or intracerebral or subarachnoid hemorrhage, the system provides a blood oxygen saturation monitor, using an infra-red or laser source, to alert the user if the patient's blood in the brain or some brain region is deoxygenated.

A stimulus device may optionally be placed on each subject, such as an audio generator in the form of an ear plug, which produces a series of “click” sounds. The subject's brain waves are detected and converted into audio tones. The device may have an array of LED (Light Emitting Diodes) which blink depending on the power and frequency composition of the brain wave signal. Power ratios in the frequencies of audio or somatosensory stimuli are similarly encoded. The EEG can be transmitted to a remote physician or medical aide who is properly trained to determine whether the patient's brain function is abnormal and may evaluate the functional state of various levels of the patient's nervous system.

In another embodiment, three pairs of electrodes are attached to the head of the subject under examination via tape or by wearing a cap with electrodes embedded. In one embodiment, the electrode pairs are as follows:
1) top of head to anterior throat
2) inion-nasion
3) left to right mastoid (behind ear).

A ground electrode is located at an inactive site of the upper part of the vertebral column. The electrodes are connected to differential amplification devices as disclosed below. Because the electrical charges of the brain are so small (on the order of microvolts), amplification is needed. The three amplified analog signals are converted to digital signals and averaged over a certain number of successive digital values to eliminate erroneous values originated by noise on the analog signal.

All steps defined above are linked to a timing signal which is also responsible for generating stimuli to the subject. The responses are processed in a timed relation to the stimuli and averaged as the brain responds to these stimuli. Of special interest are the responses within certain time periods and time instances after the occurrence of a stimulus of interest. These time periods and instances and their references can be:
25 to 60 milliseconds: P1-N1
180 to 250 milliseconds: N2
100 milliseconds: N100
200 milliseconds: P2
300 milliseconds: P300.

In an examination two stimuli sets may be used in a manner that the brain has to respond to the two stimuli differently, one stimulus has a high probability of occurrence, and the other stimulus is a rare occurring phenomena. The rare response is the response of importance. Three response signals are sensed and joined into a three dimensional cartesian system by a mapping program. The assignments can be
nasion-inion=X,
left-right mastoid=Y, and
top of head to anterior throat=Z.

The assignment of the probes to the axes and the simultaneous sampling of the three response signals at the same rate and time relative to the stimuli allows to real-time map the electrical signal in a three dimensional space. The signal can be displayed in a perspective representation of the three dimensional space, or the three components of the vector are displayed by projecting the vector onto the three planes X-Y, Y-Z, and X-Z, and the three planes are inspected together or separately. Spatial information is preserved for reconstruction as a map. The Vector Amplitude (VA) measure provides information about how far from the center of the head the observed event is occurring; the center of the head being the center (0,0,0) of the coordinate system.

The cranial bioimpedance sensor can be applied singly or in combination with a cranial blood flow sensor, which can be optical, ultrasound, electromagnetic sensor(s) as described in more details below. In an ultrasound imaging implementation, the carotid artery is checked for plaque build-up. Atherosclerosis is systemic—meaning that if the carotid artery has plaque buildup, other important arteries, such as coronary and leg arteries, might also be atherosclerotic.

In another embodiment, an epicardial array monopolar ECG system converts signals into the multichannel spectrum domain and identifies decision variables from the autospectra. The system detects and localizes the epicardial projections of ischemic myocardial ECGs during the cardiac activation phase. This is done by transforming ECG signals from an epicardial or torso sensor array into the multichannel spectral domain and identifying any one or more of a plurality of decision variables. The ECG array data can be used to detect, localize and quantify reversible myocardial ischemia.

In yet another embodiment, a trans-cranial Doppler velocimetrysensor provides a non-invasive technique for measuring blood flow in the brain. An ultrasound beam from a transducer is directed through one of three natural acoustical windows in the skull to produce a waveform of blood flow in the arteries using Doppler sonography. The data collected to determine the blood flow may include values such as the pulse cycle, blood flow velocity, end diastolic velocity, peak systolic velocity, mean flow velocity, total volume of cerebral blood flow, flow acceleration, the mean blood pressure in an artery, and the pulsatility index, or impedance to flow through a vessel. From this data, the condition of an artery may be derived, those conditions including stenosis, vasoconstriction, irreversible stenosis, vasodilation, compensatory vasodilation, hyperemic vasodilation, vascular failure, compliance, breakthrough, and pseudo-normalization.

To detect stroke attack, the system can detect numbness or weakness of the face, arm or leg, especially on one side of the body. The system detects sudden confusion, trouble speaking or understanding, sudden trouble seeing in one or both eyes, sudden trouble walking, dizziness, loss of balance or coordination, or sudden, severe headache with no known cause. In one embodiment to detect heart attack, the system detects discomfort in the center of the chest that lasts more than a few minutes, or that goes away and comes back. Symptoms can include pain or discomfort in one or both arms, the back, neck, jaw or stomach. The system can also monitor for shortness of breath which may occur with or without chest discomfort. Other signs may include breaking out in a cold sweat, nausea or lightheadedness. In order to best analyze a patient's risk of stroke, additional patient data is utilized by a stroke risk analyzer. This data may include personal data, such as date of birth, ethnic group, sex, physical activity level, and address. The data may further include clinical data such as a visit identification, height, weight, date of visit, age, blood pressure, pulse rate, respiration rate, and so forth. The data may further include data collected from blood work, such as the antinuclear antibody panel, B-vitamin deficiency, C-reactive protein value, calcium level, cholesterol levels, entidal CO2, fibromogin, amount of folic acid, glucose level, hematocrit percentage, H-pylori antibodies, hemocysteine level, hypercapnia, magnesium level, methyl maloric acid level, platelets count, potassium level, sedrate (ESR), serum osmolality, sodium level, zinc level, and so forth. The data may further include the health history data of the patient, including alcohol intake, autoimmune diseases, caffeine intake, carbohydrate intake, carotid artery disease, coronary disease, diabetes, drug abuse, fainting, glaucoma, head injury, hypertension, lupus, medications, smoking, stroke, family history of stroke, surgery history, for example. The automated analyzer can also consider related pathologies in analyzing a patient's risk of stroke, including but not limited to gastritis, increased intracranial pressure, sleep disorders, small vessel disease, and vasculitis.

FIG. 5F shows an exemplary band-aid or patch with flexible circuits thereon. The patch may be applied to a persons skin by anyone including the person themselves or an authorized person such as a family member or physician. The adhesive patch can have a gauze pad attached to one side of a backing, preferably of plastic, and wherein the pad can have an impermeable side coating with backing and a module which contains electronics for communicating with the mesh network and for sensing acceleration and bioimpedance, EKG/ECG, heart sound, microphone, optical sensor, or ultrasonic sensor in contacts with a wearer's skin. In one embodiment, the module has a skin side that may be coated with a conductive electrode lotion or gel to improve the contact. The entire patch described above may be covered with a plastic or foil strip to retain moisture and retard evaporation by a conductive electrode lotion or gel provided improve the electrode contact. In one embodiment, an acoustic sensor (microphone or piezoelectric sensor) and an electrical sensor such as EKG sensor contact the patient with a conductive gel material. The conductive gel material provides transmission characteristics so as to provide an effective acoustic impedance match to the skin in addition to providing electrical conductivity for the electrical sensor. The acoustic transducer can be directed mounted on the conductive gel material substantially with or without an intermediate air buffer. The entire patch is then packaged as sterile as are other over-the-counter adhesive bandages. When the patch is worn out, the module may be removed and a new patch backing may be used in place of the old patch. One or more patches may be applied to the patient's body and these patches may communicate wirelessly using the mesh network or alternatively they may communicate through a personal area network using the patient's body as a communication medium.

The term “positional measurement,” as that term is used herein, is not limited to longitude and latitude measurements, or to metes and bounds, but includes information in any form from which geophysical positions can be derived. These include, but are not limited to, the distance and direction from a known benchmark, measurements of the time required for certain signals to travel from a known source to the geophysical location where the signals may be electromagnetic or other forms, or measured in terms of phase, range, Doppler or other units

FIG. 5G shows an exemplary contact lens with flexible circuits thereon and FIG. 5H shows an exemplary eye glass with flexible circuits thereon. The contact lens can detect glucose levels using the sensors detailed above. In addition, the contact lens can be placed on eyeglasses to provide augmented reality. The contact lens or a sunglass or eyeglass embodiment contains electronics for communicating with the mesh network and for sensing acceleration and bioimpedance, EKG/ECG, EMG, heart sound, microphone, optical sensor, or ultrasonic sensor in contacts with a wearer's skin. In one embodiment, the ear module contains optical sensors to detect temperature, blood flow and blood oxygen level as well as a speaker to provide wireless communication or hearing aid. The blood flow or velocity information can be used to estimate blood pressure. The side module can contain an array of bioimpedance sensors such as bipolar or tetrapolarbioimpedance probes to sense fluids in the brain. Additional bioimpedance electrodes can be positioned around the rim of the glasses as well as the glass handle or in any spots on the eyewear that contacts the user. The side module can also contain one or more EKG electrodes to detect heart beat parameters and to detect heart problems. The side module can also contain piezoelectric transducers or microphones to detect heart activities near the brain. The side module can also contain ultrasound transmitter and receiver to create an ultrasound model of brain fluids. In one embodiment, an acoustic sensor (microphone or piezoelectric sensor) and an electrical sensor such as EKG sensor contact the patient with a conductive gel material. The conductive gel material provides transmission characteristics so as to provide an effective acoustic impedance match to the skin in addition to providing electrical conductivity for the electrical sensor. The acoustic transducer can be directed mounted on the conductive gel material substantially with or without an intermediate air buffer. In another embodiment, electronics components are distributed between first and second ear stems. In yet another embodiment, the method further comprises providing a nose bridge, wherein digital signals generated by the electronics circuit are transmitted across the nose bridge. The eyewear device may communicate wirelessly using the mesh network or alternatively they may communicate through a personal area network using the patient's body as a communication medium. Voice can be transmitted over the mesh wireless network. The speaker can play digital audio file, which can be compressed according to a compression format. The compression format may be selected from the group consisting of: PCM, DPCM, ADPCM, AAC, RAW, DM, RIFF, WAV, BWF, AIFF, AU, SND, CDA, MPEG, MPEG-1, MPEG-2, MPEG-2.5, MPEG-4, MPEG-J, MPEG 2-ACC, MP3, MP3Pro, ACE, MACE, MACE-3, MACE-6, AC-3, ATRAC, ATRAC3, EPAC, Twin VQ, VQF, WMA, WMA with DRM, DTS, DVD Audio, SACD, TAC, SHN, OGG, OggVorbis, OggTarkin, OggTheora, ASF, LQT, QDMC, Alb, .ra, .rm, and Real Audio G2, RMX formats, Fairplay, Quicktime, SWF, and PCA, among others.

In one embodiment, the eye wear device can provide a data port, wherein the data port is carried by the ear stem. The data port may be a mini-USB connector, a FIREWIRE connector, an IEEE 1394 cable connector, an RS232 connector, a JTAB connector, an antenna, a wireless receiver, a radio, an RF receiver, or a Bluetooth receiver. In another embodiment, the wearable device is removably connectable to a computing device. The wearable wireless audio device may be removably connectable to a computing device with a data port, wherein said data port is mounted to said wearable wireless audio device. In another embodiment, projectors can project images on the glasses to provide head-mounted display on the eye wear device. The processor can display fact, figure, to do list, and reminders need in front of the user's eyes.

FIGS. 5I-5J shows an exemplary quality assurance system for vegetable or medication packages that need to monitor a temperature range, for example. The QA system incorporates a temperature sensor, a shock sensor (for eggs/medication), a display with a wireless communication system and energy scavenger or battery to operate the unit, all of which can be formed as detailed above. In certain embodiments, functionalized CNT sensor can be used to detect biohazards such as bacteria and contaminants. The system enables a Sanitary Transport of Human & Animal Food. The wireless RFID provides in-depth record keeping, expanded maintenance of the cold chain, more detailed procedures for loading and unloading, and standard processes for the exchange of information. The system ensures that the temperatures to which foods are subjected during storage, transportation and in processing are within specifications. RFID is used in food is in three areas: using UHF RFID to improve delivery accuracy for perishable foods in the distribution center (DC); using UHF RFID to better manage food shelf life and waste in grocery stores/supermarkets; and combining NFC with time-temperature sensors to monitor the food cold chain. He added that RFID is being used in-store for preventing food waste, primarily for meat and ready-made meals, and for perishable foods in the DC.

Flexible electronic labels are placed on perishable loads to monitor temperatures. When the loads are received, the labels are automatically detected by a reader and the cold chain data is automatically forwarded to pre-defined users via email or text. The data delivered includes the supplier name, product description, temperature alert condition, receiving location, high/low/average temperatures, and a temperature graph. Data can also be forwarded to a central repository for ongoing carrier, supplier and route analysis as part of an overall business intelligence strategy. Food and drug monitoring will become more customer-facing over time, as consumers want to know where their food and medicine come from and how it is processed. The flexible RFID gives retailers the opportunity to check that they have the right products, the right quantities and most importantly, the right dates, at every stage of the chain, at minimum labor costs.

FIG. 5K shows an exemplary large panel with flexible resistive heater circuits thereon. The resistive heater can be placed on the clothing to provide warmth if needed. The panel can also be formed into seats. One embodiment can be used as windshield defrosters for cars or for large windows in a house.

FIG. 5L shows an exemplary active display billboard with flexible circuits thereon. The flexible display can also be used as a large window that displays virtual locations. For example, the user may instruct the display to show his/her favorite vacation locations as a video on the wall so that the user can experience the remote location without leaving home or office.

FIG. 5M shows exemplary carpet or floor tiles with flexible circuits thereon. In one embodiment, the carpet or the floor tiles (wood or laminate) contains pressure sensors that can detect footsteps and user movement in the house for activity monitoring. Additionally, the floor tiles can contain LEDs so that in case of an emergency, the LEDs point the way to exhibit and avoid danger. Moreover, if the carpet/tile is soiled, the system can automatically call for cleaning or alternatively, notify the user that the carpet/tile may need replacement.

FIGS. 5N-5O show exemplary smart building exterior with flexible circuits thereon. In these embodiments, the exterior is a display that, with a camera, captures its environment and then displays the environment to blend in with the environment, and in full blend mode, can become “invisible” because the displays show images blocked by the conventional exterior material. This concept can be used inside the home to create an illusion of more space, for example.

In a general sense, those having ordinary skill in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those having ordinary skill in the art will also appreciate that examples of electro-mechanical systems include but are not limited to a variety of consumer electronics systems, medical devices, as well as other systems such as motorized transport systems, factory automation systems, security systems, and/or communication/computing systems. Those having ordinary skill in the art will recognize that electro-mechanical as used herein is not necessarily limited to a system that has both electrical and mechanical actuation except as context may dictate otherwise.

In a general sense, those having ordinary skill in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

In some instances, one or more components may be referred to herein as “configured to,” “configurable to,” “operable/operative to,” “adapted/adaptable,” “able to,” “conformable/conformed to,” etc. Those having ordinary skill in the art will recognize that “configured to” can generally encompass active-state components and/or inactive-state components and/or standby-state components, unless context requires otherwise. While particular aspects of the present subject matter described herein have been shown and described, it will be apparent to those having ordinary skill in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those having ordinary skill in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms. For example, the phrase “A or B” will be typically understood to include the possibilities of “A” or “B” or “A and B.”

With respect to the appended claims, those having ordinary skill in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method to fabricate biodegradable electronics, comprising:
forming a biodegradable substrate;
forming a biodegradable power; and
forming a biodegradable processor, memory, and a wireless or optical communication circuit on the substrate.

2. The system of claim 1, wherein the biodegradeable substrate comprises a biodegradable paper or biodegradable polymer.

3. The method of claim 1, comprising printing or depositing components above the substrate.

4. The method of claim 1, wherein the processor and wireless communication system can communicate with a remote computer to provide information about the source of the items

5. The method of claim 1, comprising storing supply chain information on the substrate.

6. The method of claim 1, comprising storing production or expiration information on the substrate.

7. The method of claim 1, comprising storing tamper-proof information on the substrate.

8. The method of claim 1, comprising storing blockchain information on the substrate.

9. The method of claim 1, comprising forming a biodegradable display on the substrate.

10. The method of claim 1, comprising forming a bacteria storage on the substrate.

11. The method of claim 10, comprising forming a biodegradable food storage coupled to the bacteria storage on the substrate.

12. The method of claim 10, comprising forming a liquid storage coupled to the bacteria storage on the substrate, wherein liquid is selectively introduced to the bacteria to activate the bacteria to provide energy.

13. The method of claim 1, comprising detecting a predetermined substance.

14. The method of claim 13, comprising:
functionalizing a macromolecule with a material to couple to a predetermined substance;
forming the functionalized macromolecule on the substrate;
exposing the macromolecule to an operating environment to attach the macromolecule to the predetermined substance;
measuring an electrical characteristic indicative of the presence of the predetermined substance; and
indicating a presence of the substance if the electrical characteristic is greater than or less than a predetermined range.

15. The method of claim 14, comprising securing the macromolecule to a skin to capture sweat.

16. The method of claim 13, comprising detecting one or more of metabolite, glucose, lactate, electrolyte, sodium, potassium.

17. The method of claim 13, wherein the substance comprises a bio-marker, comprising exposing the macromolecule to blood.

18. The method of claim 13, comprising securing a macromolecule to a skin with microneedles to expose the macromolecule to subdermal blood.

19. The method of claim 18, comprising forming an implantable medical device with an exposed region to expose the macromolecule to blood and implanting the device inside a person.

20. The method of claim 17, wherein the bio-marker comprises one or more cancer biomarkers, further comprising detecting cancer from DNA fragments circulating in the blood and wherein the material comprises a predetermined DNA sequence, further comprising:
functionalizing the macromolecule with a second material to bond with a second DNA sequence complementary to the predetermined DNA sequence;
during operation, generating a complementary DNA sequence from cell material in the blood and coupling the complementary DNA sequence to the second material;
characterizing a second electrical characteristic indicative of the presence of the second DNA sequence,
applying differential analysis to the first and second electrical characteristics to accurately determine a presence of the predetermined DNA sequence; and
detecting the presence of the bio-marker using machine learning.
